(19) United States
(12) Patent Application Publication
(10) Pub. No.: US 2024/0043543 A1
(43) Pub. Date: Feb. 8, 2024

(54) ANTI-GALECTIN-9 ANTIBODIES AND THERAPEUTIC USES THEREOF

(71) Applicants: New York University, New York, NY (US); PureTech LYT, Inc., Boston, MA (US)

(72) Inventors: Shohei KOIDE, New York, NY (US); Akiko KOIDE, New York, NY (US); Aleksandra FILIPOVIC, Boston, MA (US); Eric ELENKO, Boston, MA (US); Joseph BOLEN, Boston, MA (US)

(21) Appl. No.: 18/253,754

(22) PCT Filed: Nov. 19, 2021
(86) PCT No.: PCT/US2021/060143

§ 371 Date: May 19, 2023

Related U.S. Application Data
(60) Provisional application No. 63/116,553, filed on Nov. 20, 2020.

(51) Int. Cl. C07K 16/28; A61P 35/04

(52) U.S. Cl. CPC C07K 16/2851; A61P 35/04; C07K 2317/565; A61K 2039/545

(57) ABSTRACT

Combined therapy for a solid tumor, comprising an antibody that binds human galectin-9 (anti-Gal9 antibody, e.g., G9.2-17), and one or more chemotherapeutics, for example, gemcitabine, paclitaxel, or a combination thereof.

### CROSS REFERENCE TO RELATED APPLICATIONS

This application claims the benefit under 35 U.S.C. § 119(e) of U.S. Provisional Application No. 63/116,553, filed Nov. 20, 2020, the contents of which are incorporated by reference herein in their entirety.

### BACKGROUND OF INVENTION

Galectin-9 is a tandem-repeat lectin consisting of two carbohydrate recognition domains (CRDs) and was discovered and described for the first time in 1997 in patients suffering from Hodgkin's lymphoma (HL) (Tureci et al., J. Biol. Chem. 1997, 272, 6416-6422). Three isoforms exist, and can be located within the cell or extracellularly. Elevated Galectin-9 levels have been in observed a wide range of cancers, including melanoma, Hodgkin's lymphoma, hepatocellular, pancreatic, gastric, colon and clear cell renal cell cancers (Wdowiak et al. Int. J. Mol. Sci. 2018, 19, 210). In renal cancer, patients with high Galectin-9 expression showed more advanced progression of the disease with larger tumor size (Kawashima et al.; BJU Int. 2014; 113:320-332). In melanoma, Galectin-9 was expressed in 57% of tumors and was significantly increased in the plasma of patients with advanced melanoma compared to healthy controls (Enninga et al., Melanoma Res. 2016 October; 26(5): 429-441). A number of studies have shown utility for Galectin-9 as a prognostic marker, and more recently as a potential new drug target (Enninga et al., 2016; Kawashima et al. BJU Int 2014; 113: 320-332; Kageshita et al., Int J Cancer. 2002 Jun. 20; 99(6):809-16, and references therein).

Galectin-9 has been described to play an important role in in a number of cellular processes such as adhesion, cancer cell aggregation, apoptosis, and chemotaxis. Recent studies have shown a role for Galectin-9 in immune modulation in support of the tumor, e.g., through negative regulation of Th1 type responses, Th2 polarization and polarization of macrophages to the M2 phenotype. This work also includes studies that have shown that Galectin-9 participates in direct inactivation of T cells through interactions with the T-cell immunoglobulin and mucin protein 3 (TIM-3) receptor (Dardalhon et al., J Immunol., 2010, 185, 1383-1392; Sanchez-Fueyo et al., Nat Immunol., 2003, 4, 1093-1101).

Galectin-9 has also been found to play a role in polarizing T cell differentiation into tumor suppressive phenotypes), as well as promoting tolerogenic macrophage programming and adaptive immune suppression (Daley et al., Nat Med., 2017, 23, 556-567). In mouse models of pancreatic ductal adenocarcinoma (PDA), blockade of the checkpoint interaction between Galectin-9 and the receptor Dectin-1 found on innate immune cells in the tumor microenvironment (TME) has been shown to increase anti-tumor immune responses in the TME and to slow tumor progression (Daley et al., Nat Med., 2017, 23, 556-567). Galectin-9 also has been found to bind to CD206, a surface marker of M2 type macrophages, resulting in a reduced secretion of CVL22 (MDC), a macrophage derived chemokine which has been associated with longer survival and lower recurrence risk in lung cancer (Enninga et al, J Pathol. 2018 August; 245(4):468-477).

### SUMMARY OF INVENTION

The present disclosure is based on the unexpected discovery that a synergistic effect is observed in combined therapies involving both an exemplary anti-galectin 9 antibody (e.g., G9.2-17(IgG4)) and chemotherapeutics such as gemcitabine and paclitaxel (e.g., nanoparticle albumin-bound paclitaxel or nab-paclitaxel) in an animal model.

Accordingly, provided herein are methods for treating a solid tumor involving the co-use of an anti-galectin-9 antibody (e.g., G9.2-17 or a functional variant thereof) and one or more chemotherapeutics (e.g., gemcitabine, paclitaxel such as paclitaxel protein-bound (e.g., nab-paclitaxel or Abraxane®), or a combination thereof).

In some embodiments, the method for treating a solid tumor disclosed herein may comprise administering to a subject in need thereof an effective amount of an antibody that binds human galectin-9 (anti-Gal9 antibody). The anti-Galectin-9 antibody may have the same heavy chain complementarity determining regions (CDRs) and the same light chain CDRs as antibody G9.2-17. The subject may be undergoing an anti-cancer therapy comprising one or more chemotherapeutics.

In some embodiments, the method for treating a solid tumor disclosed herein may comprise administering to a subject in need thereof an effective amount of an antibody that binds human galectin-9 (anti-Gal9 antibody) and an effective amount of one or more chemotherapeutics. The anti-Gal9 antibody may have the same heavy chain complementarity determining regions (CDRs) and the same light chain CDRs as antibody G9.2-17.

In some embodiments, the method for treating a solid tumor disclosed herein may comprise administering to a subject in need thereof an effective amount of one or more chemotherapeutics. The subject may be undergoing a therapy comprising an antibody that binds human galectin-9 (anti-Gal9 antibody), which has the same heavy chain complementarity determining regions (CDRs) and the same light chain CDRs as antibody G9.2-17.

Any of the methods disclosed herein may be applied for treating a metastatic solid tumor. In some examples, the solid tumor is pancreatic ductal adenocarcinoma (PDAC), for example, metastatic PDAC.

In some embodiments, the subject to be treated by any of the methods disclosed herein may have one or more of the following features: (i) has no resectable cancer; (ii) has no infection by SARS-CoV-2; and (iii) has no active brain or leptomeningeal metastasis. In some examples, the solid tumor is pancreatic ductal adenocarcinoma (PDAC), and the subject has no locally advanced PDAC without distant organ metastatic deposits.

In some embodiments, the one or more chemotherapeutics involved in any of the methods disclosed herein may comprise an antimetabolite (e.g., a nucleoside analog), a microtubule inhibitor, or a combination thereof. In some examples, the nucleoside analog is gemcitabine and/or the tubulin inhibitor is paclitaxel, for example, nanoparticle albumin-bound paclitaxel (e.g., Abraxane®).

In some embodiments, the anti-Galectin-9 antibody is administered to the subject at a dose of about 0.5 mg/kg to about 32 mg/kg (e.g., about 0.5 mg/kg to about 16 mg/kg, about 2 mg/kg to about 32 mg/kg or about 2 mg/kg to about 16 mg/kg). In some embodiments, the anti-Galectin-9 antibody is administered to the subject once a week. In some embodiments, the anti-Galectin-9 antibody is administered to the subject once every 2 or 3 weeks. In some embodiments, the anti-Galectin-9 antibody is administered to the subject at a dose selected from 2 mg/kg, 4 mg/kg, 8 mg/kg, 12 mg/kg, or 16 mg/kg. In some embodiments, the antibody is administered once every 2 weeks. In some embodiments, the anti-Galectin-9 antibody is administered to the subject at a dose selected from 2 mg/kg, 4 mg/kg, 8 mg/kg, 12 mg/kg, or 16 mg/kg once every 2 weeks. In some embodiments, the anti-Galectin-9 antibody is administered once every 2 weeks for one cycle, once every 2 weeks for two cycles, once every 2 weeks for 3 cycles, once every 2 weeks for 4 cycles, or once every 2 weeks for more than 4 cycles. In some embodiments, the duration of treatment is 0-3 months, 0-6 months, 3-6 months, 6-12 months, 12-24 months or longer. In some embodiments, the duration of treatment is 12-24 months or longer. In some embodiments, the cycles extend for a duration of 3 months to 6 months, or 6 months to 12 months or 12 months to 24 months or longer. In some embodiments, the cycle length is modified, e.g., temporarily or permanently to a longer duration, e.g., 3 weeks or 4 weeks. In any of these embodiments, the anti-Galectin-9 antibody is administered to the subject once a week, once every two weeks, once every three weeks, or once every four weeks. In some embodiments, the anti-Galectin-9 antibody is administered to the subject by intravenous infusion. In some embodiments, the cancer is PDA. In some embodiments, the cancer is metastatic cancer.

In some embodiments, the anti-Gal9 antibody can be administered to the subject at a dose of about 0.5 mg/kg to about 32 mg/kg once every two weeks by intravenous injection. In some examples, the anti-Gal9 antibody is administered to the subject at a dose of about 0.5 mg/kg once every two weeks by intravenous injection. In some embodiments, the anti-Gal9 antibody can be administered to the subject at a dose of about 2 mg/kg to about 16 mg/kg once every two weeks by intravenous injection. In some examples, the anti-Gal9 antibody is administered to the subject at a dose of about 2 mg/kg once every two weeks by intravenous injection. In some examples, the anti-Gal9 antibody is administered to the subject at a dose of about 4 mg/kg once every two weeks by intravenous injection. In some examples, the anti-Gal9 antibody is administered to the subject at a dose of about 8 mg/kg once every two weeks by intravenous injection. In some examples, the anti-Gal9 antibody is administered to the subject at a dose of about 12 mg/kg once every two weeks by intravenous injection. In some examples, the anti-Gal9 antibody is administered to the subject at a dose of about 16 mg/kg once every two weeks by intravenous injection. In some examples, the anti-Gal9 antibody is administered to the subject at a dose of about 32 mg/kg once every two weeks by intravenous injection.

In some embodiments, the method comprises a cycle of 28 days, in which the anti-Gal9 antibody is administered to the subject on day 1 and day 15 and gemcitabine and paclitaxel (e.g., nanoparticle albumin-bound paclitaxel) are administered to the subject on day 1, day 8, and day 15. In some examples, the paclitaxel is administered to the subject at 125 mg/m² intravenously. In some examples, the gemcitabine is administered to the subject at 1000 mg/m².

In some embodiments, the anti-Galectin-9 antibody comprises a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3 and/or comprises a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6.

In some embodiments, the anti-Gal9 antibody may comprise a heavy chain variable region (V_{H}) that comprises the amino acid sequence of SEQ ID NO: 7; and a light chain variable region (V_{L}) that comprises the amino acid sequence of SEQ ID NO: 8. In some examples, the anti-Gal9 antibody can be an IgG molecule, for example, an IgG4 molecule. In specific examples, the anti-Gal9 antibody may comprise a heavy chain that comprises the amino acid sequence of SEQ ID NO: 19 and a light chain that comprises the amino acid sequence of SEQ ID NO: 15.

In some embodiments, the subject to be treated by any of the methods disclosed herein can be a human patient. In some examples, the subject has galectin-9 positive cancer cells or immune cells. Such galectin-9 positive cancer cells or immune cells may be detected in tumor organoids derived from the subject. In some examples, the subject may have an elevated level of galectin-9 relative to a control value. For example, the subject may have an elevated serum or plasma level of galectin-9 relative to the control value.

In some embodiments, the subject may have received at least one line of systemic anti-cancer therapy. Alternatively or in addition, the subject may be free of prior therapy involving gemcitabine and/or paclitaxel. In some examples, the subject may have received a prior therapy involving gemcitabine and/or paclitaxel at least six months before administration of the anti-Gal9 antibody.

In any of the methods disclosed herein, the subject is examined for one or more of the following features before, during, and/or after the treatment: (a) one or more tumor markers in blood samples from the subject, optionally wherein the one or more tumor markers comprise CA15-3, CA-125, CEA, CA19-9, and/or alpha fetoprotein, and any other tumor-type specific tumor markers; (b) cytokine profile; and (c) galectin 9 serum/plasma levels, d) peripheral blood mononuclear cell immunophenotyping, e) tumor tissue biopsy/excisional specimen multiplex immunophenotyping, f) tumor tissue biopsy/excisional specimen galectin-9 expression levels and pattern, g) any other immune score test such as: PDL-1 immunohistochemistry, tuor mutational burden (TMB), tumor microsatellite instability status, as well as panels such as: Immunoscore®—HalioDx, ImmunoSeq-Adaptive Biotechnologies, TIS, developed on the NanoString nCounter® gene expression system, 18-gene signature, PanCancer IO 360™ assay (NanoString Technologies) etc. Other suitable biomarkers specific to the target tumor may also be examined.

Any of the methods disclosed herein may further comprise monitoring occurrence of one or more adverse effects in the subject. Exemplary adverse effects include, but are not limited to, hepatic impairment, hematologic toxicity, neurologic toxicity, cutaneous toxicity, gastrointestinal toxicity, or a combination thereof. When one or adverse effects are observed, the method disclosed herein may further comprise reducing the dose of the anti-Gal9 antibody, the dose of the one or more chemotherapeutics, or both. For example, when moderate to severe hepatic impairment is observed in a subject, the method may further comprise reducing the dose of the anti-Gal9 antibody, the dose of gemcitabine, the dose of paclitaxel, or a combination thereof.

In some examples, administration of the paclitaxel is withheld when the subject has a level of aspartate transaminase (AST) greater than 10× upper limit of normal (ULN), a level of bilirubin greater than 5×ULN, or both. In some embodiments, the method may further comprise reducing the dose or terminating administration of the anti-Gal9 antibody, gemcitabine, paclitaxel, or a combination thereof, when severe hematologic toxicity, neurologic toxicity, cutaneous toxicity, and/or gastrointestinal toxicity is observed.

In some examples, the dose of the paclitaxel may be reduced to 100 mg/m²-75 mg/m². Alternatively or in addition, the dose of gemcitabine is reduced to 800 mg/m²-600 mg/m².

Also within the scope of the present disclosure are pharmaceutical compositions comprising any of the anti-Gal9 antibodies and the one or more chemotherapeutics for use in treating a solid tumor such as PDAC, as well as uses of a combination of the anti-Gal9 antibody and the one or more chemotherapeutic agents for manufacturing a medicament for use in treating the solid tumor.

The details of one or more embodiments of the invention are set forth in the description below. Other features or advantages of the present invention are be apparent from the following drawing and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to the drawing in combination with the detailed description of specific embodiments presented herein.

FIGS. 1A-1D include graphs showing Kaplan-Meier survival curves and log rank tests for orthotopic mPA6115 pancreatic cancer xenograft mouse models grouped by treatment regimens. Group 1=untreated; Group 2=chemo vehicle control, saline; Group 3=Isotype IgG1 mouse; Group 4=Anti-Gal9 mAb; Group 5=Gemcitabine/Abraxane; and Group 6=Anti-Gal9 mAb and Gemcitabine/Abraxane. FIG. 1A shows survival curves for all six groups. FIG. 1B shows survival curves for Groups 1, 5, and 6. FIG. 1C shows survival curves for Groups 1, 4, and 6. FIG. 1D shows survival curves for Groups 1, 4, 5, and 6.

FIG. 2 includes a graph showing hazard ratios (HR) and their 95% confidence interval (%95CI) of group 4-6 against group 1, group 2 and group 3 respectively calculated from cox-regression analysis where group 1=untreated orthotopic mPA6115 mice; group 2=chemo vehicle control, saline treated orthotopic mPA6115 mice; group 3=Isotype IgG1 mouse treated orthotopic mPA6115 mice; group 4=Anti-Gal9 mAb treated orthotopic mPA6115 mice; group 5=Gemcitabine/Abraxane treated orthotopic mPA6115 mice; and group 6=Anti-Gal9 mAb and Gemcitabine/Abraxane treated orthotopic mPA6115 mice.

FIG. 3 includes a graph the mean body weight of each treatment group as measured twice a week for the study duration where group 1=untreated orthotopic mPA6115 mice; group 2=chemo vehicle control, saline treated orthotopic mPA6115 mice; group 3=Isotype IgG1 mouse treated orthotopic mPA6115 mice; group 4=Anti-Gal9 mAb treated orthotopic mPA6115 mice; group 5=Gemcitabine/Abraxane treated orthotopic mPA6115 mice; and group 6=Anti-Gal9 mAb and Gemcitabine/Abraxane treated orthotopic mPA6115 mice.

FIG. 4 includes a graph showing the effect of G2.9-17 on TGF-beta1 secretion measurements in whole blood of an exemplary healthy human donor. TGF-beta1 release from donor cryopreserved macrophages incubated in the presence of M2 polarization cocktails. IgG4 isotype is a negative control antibody. Data represent mean+SEM of triplicate measures. Significance was determined by two-way ANOVA with Dunnett's multiple comparison test. * p<0.05

FIG. 5 includes a graph showing the effect of G2.9-17 on IL-10 secretion in whole blood of an exemplary healthy human donor. IL-10 release from donor cryopreserved macrophages incubated in the presence of M2 polarization cocktails (IL-4/IL-13 or Gal-9). IgG4 isotype is a negative control antibody. Data represent the mean (±SEM) of triplicate. Significance was determined by two-way ANOVA with Tukey's multiple comparisons test, * P<0.05.

### DETAILED DESCRIPTION OF INVENTION

Provided herein are methods of co-using anti-Galectin-9 antibodies, e.g., G9.2-17, and chemotherapeutics such as gemcitabine and paclitaxel (e.g., protein-bound paclitaxel such as nanoparticle albumin-conjugated paclitaxel, for example, Abraxane®) for treating solid tumors, for example, pancreatic adenocarcinoma (PDA). In some embodiments, the solid tumors are metastatic. In some embodiments, the methods disclosed herein provide specific doses and/or dosing schedules. In some instances, the methods disclosed herein target specific patient populations, for example, patients who have undergone prior treatment and show disease progression through the prior treatment, or patients who are resistant (de novo or acquired) to the prior treatment.

Galectin-9, a tandem-repeat lectin, is a beta-galactoside-binding protein, which has been shown to have a role in modulating cell-cell and cell-matrix interactions. It is found to be strongly overexpressed in Hodgkin's disease tissue and in other pathologic states. It has in some instances also been found circulating in the tumor microenvironment (TME).

Galectin-9 interacts with Dectin-1, an innate immune receptor which is highly expressed on macrophages in PDA, as well as on cancer cells (Daley, et al. Nat Med. 2017; 23(5):556-6). Regardless of the source of Galectin-9, disruption of its interaction with Dectin-1 has been shown to lead to the reprogramming of CD4⁺ and CD8⁺ cells into indispensable mediators of anti-tumor immunity. Thus, Galectin-9 serves as a valuable therapeutic target for blocking the signaling mediated by Dectin-1. Accordingly, in some embodiments, the anti-Galectin-9 antibodies describe herein disrupt the interaction between Galectin-9 and Dectin-1.

Galectin-9 also interacts with TIM-3, a type I cell surface glycoprotein expressed on the surface of leukemic stem cells in all varieties of acute myeloid leukemia (except for M3 (acute promyelocytic leukemia)), but not expressed in normal human hematopoietic stem cells (HSCs). TIM-3 signaling resulting from Galectin-9 ligation has been found to have a pleiotropic effect on immune cells, inducing apoptosis in Th1 cells (Zhu et al., Nat Immunol., 2005, 6:1245-1252) and stimulating the secretion of tumor necrosis factor-α (TNF-α), leading to the maturation of monocytes into dendritic cells, resulting in inflammation by innate immunity (Kuchroo et al., Nat Rev Immunol., 2008, 8:577-580). Further Galectin-9/TIM-3 signaling has been found to co-activate NF-κB and β-catenin signaling, two pathways that promote LSC self-renewal (Kikushige et al., Cell Stem Cell, 2015, 17(3):341-352). An anti-Galectin-9 antibody that interferes with Galectin-9/TIM-3 binding could have a therapeutic effect, especially with respect to leukemia and other hematological malignancies. Accordingly, in some embodiments, the anti-Galectin-9 antibodies described herein disrupt the interaction between Galectin-9 and TIM-3.

Further, Galectin-9 interacts with CD206, a mannose receptor highly expressed on M2 polarized macrophages, thereby promoting tumor survival (Enninga et al., J Pathol. 2018 August; 245(4):468-477). Tumor-associated macrophages expressing CD206 are mediators of tumor immunosuppression, angiogenesis, metastasis, and relapse (see, e.g., Scodeller et al., Sci Rep. 2017 Nov. 7; 7(1):14655, and references therein). Specifically, M1 (also termed classically activated macrophages) are trigged by Th1-related cytokines and bacterial products, express high levels of IL-12, and are tumoricidal. By contrast, M2 (so-called alternatively activated macrophages) are activated by Th2-related factors, express high level of anti-inflammatory cytokines, such as IL-10, and facilitate tumor progression (Biswas and Mantovani; Nat Immunol. 2010 October; 11(10):889-96). The pro-tumoral effects of M2 include the promotion of angiogenesis, advancement of invasion and metastasis, and the protection of the tumor cells from chemotherapy-induced apoptosis (Hu et al., Tumour Biol. 2015 December; 36(12): 9119-9126, and references therein). Tumor-associated macrophages are thought be of M2-like phenotype and have a protumor role. Galectin-9 has been shown to mediate myeloid cell differentiation toward an M2 phenotype (Enninga et al., Melanoma Res. 2016 October; 26(5):429-41). It is possible that Galectin-9 binding CD206 may result in reprogramming TAMs towards the M2 phenotype, similar to what has been previously shown for Dectin. Without wishing to be bound by theory, blocking the interaction of Galectin-9 with CD206 may provide one mechanism by which an anti-Galectin-9 antibody, e.g., a G9.2-17 antibody, can be therapeutically beneficial. Accordingly, in some embodiments, the anti-Galectin-9 antibodies described herein disrupt the interaction between Galectin-9 and CD206.

Galectin-9 has also been shown to interact with protein disulfide isomerase (PDI) and 4-1BB (Bi S, et al. Proc Natl AcadS ci USA. 2011; 108(26):10650-5; Madireddi et al. J Exp Med. 2014; 211(7):1433-48).

Anti-Galectin-9 antibodies can serve as therapeutic agents for treating diseases associated with Galectin-9 (e.g., those in which a Galectin-9 signaling plays a role). Without being bound by theory, an anti-Galectin-9 antibody may block a signaling pathway mediated by Galectin-9. For example, the antibody may interfere with the interaction between Galectin-9 and its binding partner (e.g., Dectin-1, TIM-3 or CD206), thereby blocking the signaling triggered by the Galectin-9/Ligand interaction. Alternatively, or in addition, an anti-Galectin-9 antibody may also exert its therapeutic effect by inducing blockade and/or cytotoxicity, for example, ADCC, CDC, or ADCP against pathologic cells that express Galectin-9. A pathologic cell refers to a cell that contributes to the initiation and/or development of a disease, either directly or indirectly.

The anti-Galectin-9 antibodies disclosed herein are capable of suppressing the signaling mediated by Galectin-9 (e.g., the signaling pathway mediated by Galectin-9/Dectin-1 or Galectin-9/Tim-3) or eliminating pathologic cells expressing Galectin-9 via, e.g., ADCC. Accordingly, the anti-Galectin-9 antibodies described herein can be used for inhibiting any of the Galectin-9 signaling and/or eliminating Galectin-9 positive pathologic cells, thereby benefiting treatment of diseases associated with Galectin-9. See, e.g., WO2019/084553, PCT/US2020/024767, and PCT/US2020/031181, the relevant disclosures of each of which are incorporated by reference for the purpose and subject matter referenced herein.

As reported herein, combined therapy of a representative anti-Gal9 antibody (G9.2-17) and chemotherapeutics (gemcitabine and nab-paclitaxel) successfully prolonged survival in an animal model as disclosed herein. A synergistic effect of the representative anti-Gal9 antibody and gemcitabine and nab-paclitaxel on time of survival was observed in the animal model. These results demonstrate that the anti-tumor methods disclosed herein, involving the combination of an anti-Galectin-9 antibody and chemotherapeutics such as those disclosed herein, would achieve superior therapeutic efficacy than the antibody or chemotherapy alone against the target solid tumors.

Accordingly, described herein are therapeutic uses of anti-Galectin-9 antibodies and chemotherapeutics for treating certain solid tumors as disclosed herein.

### Antibodies Binding to Galectin-9

The present disclosure provides anti-Galectin-9 antibody G9.2-17 and functional variants thereof for use in the treatment methods disclosed herein.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Galectin-9 antibody, encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, the EU definition, the “Contact” numbering scheme, the IMGT” numbering scheme, the “AHo” numbering scheme, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63(1):78-85; and Almagro, J. Mol. Recognit. 17:132-143 (2004); MacCallum et al., J. Mol. Biol. 262:732-745 (1996), Lefranc M P et al., Dev Comp Immunol, 2003 January; 27(1):55-77; and Honegger A and Pluckthun A, J Mol Biol, 2001 Jun. 8; 309(3):657-70. See also hgmp.mrc.ac.uk and bioinf.org.uk/abs).

In some embodiments, the anti-Galectin-9 antibody described herein is a full-length antibody, which contains two heavy chains and two light chains, each including a variable domain and a constant domain. Alternatively, the anti-Galectin-9 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-Galectin-9 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

Reference antibody G9.2-17 refers to an antibody capable of binding to human Galectin-9 and comprises a heavy chain variable region of SEQ ID NO:7 and a light chain variable domain of SEQ ID NO:8, both of which are provided below. In some embodiments, the anti-Galectin-9 antibody for use in the methods disclosed herein is the G9.2-17 antibody. In some embodiments, the anti-Galectin-9 antibody for use in the methods disclosed herein is an antibody having the same heavy chain complementarity determining regions (CDRs) as reference antibody G9.2-17 and/or the same light chain complementarity determining regions as reference antibody G9.2-17. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/).

The heavy and light chain CDRs of reference antibody G9.2-17 is provided in Table 1 below (determined using the Kabat methodology):

TABLE 1
Heavy and Light Chain CDRs of G9.2-17

| | | | |
|---|---|---|---|
| G9.2-17 | V_{L} CDR1 | RASQSVSSAVA | SEQ ID NO: 1 |
| | V_{L} CDR2 | SASSLYS | SEQ ID NO: 2 |
| | V_{L} CDR3 | QQSSTDPIT | SEQ ID NO: 3 |
| | V_{H} CDR1 | FTVSSSSIH | SEQ ID NO: 4 |
| | V_{H} CDR2 | YISSSSGYTYYADSVKG | SEQ ID NO: 5 |
| | V_{H} CDR3 | YWSYPSWWPYRGMDY | SEQ ID NO: 6 |

In some examples, the anti-Galectin-9 antibody for use in the methods disclosed herein may comprise (following the Kabat scheme) a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6 and/or may comprise a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3. The anti-Galectin-9 antibody, including the reference antibody G9.2-17, can be in any format as disclosed herein, for example, a full-length antibody or a Fab. The term “G9.2-17(Ig4)” used herein refers to a G9.2-17 antibody which is an IgG4 molecule. Likewise, the term “G9.2-17 (Fab)” refers to a G9.2-17 antibody, which is a Fab molecule.

In some embodiments, the anti-Galectin-9 antibody or binding portion thereof comprises heavy and light chain variable regions, wherein the light chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the light chain variable region CDR1, CDR2, and CDR3 amino acid sequences set forth in SEQ ID NOs: 1, 2, and 3, respectively. In some embodiments, the anti-Galectin-9 antibody or binding portion thereof comprises heavy and light chain variable regions, wherein the heavy chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the heavy chain variable region CDR1, CDR2, and CDR3 amino acid sequences set forth in SEQ ID NO: 4, 5, and 6, respectively.

Additional Galectin-9 antibodies, e.g., which bind to the CRD1 and/or CRD2 region of Galectin-9 are described in co-owned, co-pending U.S. patent application Ser. No. 16/173,970 and in co-owned, co-pending International Patent Applications PCT/US18/58028 and PCT/US2020/024767, the contents of each of which are herein incorporated by reference in their entireties.

In some embodiments, the anti-Galectin-9 antibody disclosed herein comprises light chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{L} CDRs of reference antibody G9.2-17. Alternatively or in addition, in some embodiments, the anti-Galectin-9 antibody comprises heavy chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{H} CDRs of reference antibody G9.2-17.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-Galectin-9 antibody described herein comprises a V_{H} that comprises the HC CDR1, HC CDR2, and HC CDR3, which collectively contain up to 8 amino acid residue variations (8, 7, 6, 5, 4, 3, 2, or 1 variations(s), including additions, deletions, and/or substitutions) relative to the HC CDR1, HC CDR2, and HC CDR3 of reference antibody G9.2-17. Alternatively or in addition, in some embodiments, the anti-Galectin-9 antibody described herein comprises a V_{H} that comprises the LC CDR1, LC CDR2, and LC CDR3, which collectively contain up to 8 amino acid residue variations (8, 7, 6, 5, 4, 3, 2, or 1 variations(s) including additions, deletions, and/or substitutions) relative to the LC CDR1, LC CDR2, and LC CDR3 of reference antibody G9.2-17.

In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-Galectin-9 antibodies disclosed herein, having the heavy chain CDRs disclosed herein, contains framework regions derived from a subclass of germline V_{H} fragment. Such germline V_{H} regions are well known in the art. See, e.g., the IMGT database (www.imgt.org) or at www.vbase2.org/vbstat.php. Examples include the IGHV1 subfamily (e.g., IGHV1-2, IGHV1-3, IGHV1-8, IGHV1-18, IGHV1-24, IGHV1-45, IGHV1-46, IGHV1-58, and IGHV1-69), the IGHV2 subfamily (e.g., IGHV2-5, IGHV2-26, and IGHV2-70), the IGHV3 subfamily (e.g., IGHV3-7, IGHV3-9, IGHV3-11, IGHV3-13, IGHV3-15, IGHV3-20, IGHV3-21, IGHV3-23, IGHV3-30, IGHV3-33, IGHV3-43, IGHV3-48, IGHV3-49, IGHV3-53, IGHV3-64, IGHV3-66, IGHV3-72, and IGHV3-73, IGHV3-74), the IGHV4 subfamily (e.g., IGHV4-4, IGHV4-28, IGHV4-31, IGHV4-34, IGHV4-39, IGHV4-59, IGHV4-61, and IGHV4-B), the IGHV subfamily (e.g., IGHV5-51, or IGHV6-1), and the IGHV7 subfamily (e.g., IGHV7-4-1).

Alternatively or in addition, in some embodiments, the anti-Galectin-9 antibody, having the light chain CDRs disclosed herein, contains framework regions derived from a germline Vκ fragment. Examples include an IGKV1 framework (e.g., IGKV1-05, IGKV1-12, IGKV1-27, IGKV1-33, or IGKV1-39), an IGKV2 framework (e.g., IGKV2-28), an IGKV3 framework (e.g., IGKV3-11, IGKV3-15, or IGKV3-20), and an IGKV4 framework (e.g., IGKV4-1). In other instances, the anti-Galectin-9 antibody comprises a light chain variable region that contains a framework derived from a germline Vλ fragment. Examples include an IGλ1 framework (e.g., IGλV1-36, IGλV1-40, IGλV1-44, IGλV1-47, IGV1-51), an IGλ2 framework (e.g., IGλV2-8, IGλV2-11, IGλV2-14, IGλV2-18, IGλV2-23,), an IGλ3 framework (e.g., IGλV3-1, IGλV3-9, IGλV3-10, IGλV3-12, IGλV3-16, IGλV3-19, IGλV3-21, IGλV3-25, IGλV3-27,), an IGλ4 framework (e.g., IGλV4-3, IGλV4-60, IGλV4-69,), an IGλ5 framework (e.g., IGλV5-39, IGλV5-45,), an IGλ6 framework (e.g., IGλV6-57,), an IGλ7 framework (e.g., IGλV7-43, IGλV7-46,), an IGλ8 framework (e.g., IGV8-61), an IGλ9 framework (e.g., IGλV9-49), or an IGλ10 framework (e.g., IGλV10-54).

In some embodiments, the anti-Galectin-9 antibody for use in the method disclosed herein can be an antibody having the same heavy chain variable region (V_{H}) and/or the same light chain variable region (V_{L}) as reference antibody G9.2-17, the V_{H} and V_{L} region amino acid sequences are provided below:

V_{H}:
(SEQ ID NO: 7)
EVQLVESGGGLVQPGGSLRLSCAASGFTVSSSSIHWVRQAPGKGLEWVA
YISSSSGYTYYADSVKGRFTISADTSKNTAYLQMNSLRAEDTAVYYCAR
YWSYPSWWPYRGMDYWGQGTLVTVSS
V_{L}:
(SEQ ID NO: 8)
DIQMTQSPSSLSASVGDRVTITCRASQSVSSAVAWYQQKPGKAPKLLIY
SASSLYSGVPSRESGSRSGTDFTLTISSLQPEDFATYYCQQSSTDPITF
GQGTKVEIKR

In some embodiments, the anti-Galectin-9 antibody has at least 80% sequence identity (e.g., 80%, 85%, 90%, 91%, 92%, 93%, 94%, 95%, 96%, 97%, 98%, or 99% identity) to the heavy chain variable region of SEQ ID NO: 7. Alternatively or in addition, the anti-Galectin-9 antibody has at least 80% sequence identity (e.g., 80%, 85%, 90%, 91%, 92%, 93%, 94%, 95%, 96%, 97%, 98%, or 99% identity) to the light chain variable region of SEQ ID NO: 8.

In some instances, the anti-Galectin-9 antibody disclosed herein is a functional variant of reference antibody G9.2-17. A functional variant can be structurally similar as the reference antibody (e.g., comprising the limited number of amino acid residue variations in one or more of the heavy chain and/or light chain CDRs as G9.2-17 as disclosed herein, or the sequence identity relative to the heavy chain and/or light chain CDRs of G9.2-17, or the VH and/or VL of G9.2-17 as disclosed herein) with substantially similar binding affinity (e.g., having a KD value in the same order) to human Galectin-9.

In some embodiments, the anti-Galectin-9 antibody as described herein can bind and inhibit the activity of Galectin-9 by at least 20% (e.g., 31%, 35%, 40%, 45%, 50%, 60%, 70%, 80%, 90%, 95% or greater, including any increment therein). The apparent inhibition constant (Ki^{app} or K_{i,app}), which provides a measure of inhibitor potency, is related to the concentration of inhibitor required to reduce enzyme activity and is not dependent on enzyme concentrations. The inhibitory activity of an anti-Galectin-9 antibody described herein can be determined by routine methods known in the art.

The K_{i,}^{app} value of an antibody may be determined by measuring the inhibitory effect of different concentrations of the antibody on the extent of the reaction (e.g., enzyme activity); fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{( [ E ] - [ I ] - {K}_{i}^{app} ) + \sqrt{{( [ E ] - [ I ] - {K}_{i}^{app} )}^{2} + 4 [ E ] \cdot } {K}_{i}^{app}}{2} \qquad ( Equation 1 )$$

Where A is equivalent to vₒ/E, the initial velocity (vₒ) of the enzymatic reaction in the absence of inhibitor (I) divided by the total enzyme concentration (E). In some embodiments, the anti-Galectin-9 antibody described herein has a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target antigen or antigen epitope. In some embodiments, the anti-Galectin-9 antibody has a lower Ki^{app} for a first target (e.g., the CRD2 of Galectin-9) relative to a second target (e.g., CRD1 of the Galectin-9). Differences in Ki^{app} (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some examples, the anti-Galectin-9 antibody inhibits a first antigen (e.g., a first protein in a first conformation or mimic thereof) greater relative to a second antigen (e.g., the same first protein in a second conformation or mimic thereof; or a second protein). In some embodiments, any of the anti-Galectin-9 antibodies is further affinity matured to reduce the Ki^{app} of the antibody to the target antigen or antigenic epitope thereof.

In some embodiments, the anti-Galectin-9 antibody suppresses Dectin-1 signaling, e.g., in tumor infiltrating immune cells, such as macrophages. In some embodiments, the anti-Galectin-9 antibody suppresses Dectin-1 signaling triggered by Galectin-9 by at least 30% (e.g., 31%, 35%, 40%, 50%, 60%, 70%, 80%, 90%, 95% or greater, including any increment therein). Such inhibitory activity can be determined by conventional methods, such as routine assays. Alternatively or in addition, the anti-Galectin-9 antibody suppresses the T cell immunoglobulin mucin-3 (TIM-3) signaling initiated by Galectin-9. In some embodiments, the anti-Galectin-9 antibody suppresses the T cell immunoglobulin mucin-3 (TIM-3) signaling, e.g., in tumor infiltrating immune cells, e.g., in some embodiments by at least 30% (e.g., 31%, 35%, 40%, 50%, 60%, 70%, 80%, 90%, 95% or greater, including any increment therein). Such inhibitory activity can be determined by conventional methods, such as routine assays.

In some embodiments, the anti-Galectin-9 antibody suppresses the CD206 signaling, e.g., in tumor infiltrating immune cells. In some embodiments, the anti-Galectin-9 antibody suppresses the CD206 signaling triggered by Galectin-9 by at least 30% (e.g., 31%, 35%, 40%, 50%, 60%, 70%, 80%, 90%, 95% or greater, including any increment therein). Such inhibitory activity can be determined by conventional methods, such as routine assays. In some embodiments, the anti-Galectin-9 antibody blocks or prevents binding of Galectin-9 to CD206 by at least 30% (e.g., 31%, 35%, 40%, 50%, 60%, 70%, 80%, 90%, 95% or greater, including any increment therein). Such inhibitory activity can be determined by conventional methods, such as routine assays.

In some embodiments, the anti-Galectin-9 antibody induces cell cytotoxicity, such as ADCC, in target cells expressing Galectin-9, e.g., wherein the target cells are cancer cells or immune suppressive immune cells. In some embodiments, the anti-Galectin-9 antibody induces apoptosis in immune cells, such as T cells, or cancer cells by at least 30% (e.g., 31%, 35%, 40%, 50%, 60%, 70%, 80%, 90%, 95% or greater, including any increment therein). Such inhibitory activity can be determined by conventional methods, such as routine assays. In some embodiments, any of the anti-Galectin-9 antibodies described herein induce cell cytotoxicity such as complement-dependent cytotoxicity (CDC) against target cells expressing Galectin-9.

Antibody-dependent cell-mediated phagocytosis (ADCP) is an important mechanism of action for antibodies that mediate part or all of their action though phagocytosis. In that case, antibodies mediate uptake of specific antigens by antigen presenting cells. ADCP can be mediated by monocytes, macrophages, neutrophils, and dendritic cells, through FcγRIIa, FcγRI, and FcγRIIIa, of which FcγRIIa (CD32a) on macrophages represent the predominant pathway.

In some embodiments, the anti-Galectin-9 antibody induces cell phagocytosis of target cells, e.g., cancer cells or immune suppressive immune cells expressing Galectin-9 (ADCP). In some embodiments, the anti-Galectin-9 antibody increases phagocytosis of target cells, e.g., cancer cells or immune suppressive immune cells, by at least 30% (e.g., 31%, 35%, 40%, 50%, 60%, 70%, 80%, 90%, 95% or greater, including any increment therein).

In some embodiments, the anti-Galectin-9 antibody described herein induces cell cytotoxicity such as complement-dependent cytotoxicity (CDC) against target cells, e.g., cancer cells or immune suppressive immune cells. In some embodiments, the anti-Galectin-9 antibody increases CDC against target cells by at least 30% (e.g., 31%, 35%, 40%, 50%, 60%, 70%, 80%, 90%, 95% or greater, including any increment therein).

In some embodiments, the anti-Galectin-9 antibody induces T cell activation, e.g., in tumor infiltrating T cells, i.e., suppress Galectin-9 mediated inhibition of T cell activation, either directly or indirectly. In some embodiments, the anti-Galectin-9 antibody promotes T cell activation by at least 30% (e.g., 31%, 35%, 40%, 50%, 60%, 70%, 80%, 90%, 95% or greater, including any increment therein). T cell activation can be determined by conventional methods, such as assays (e.g., measurement of CD44, TNF alpha, IFNgamma, and/or PD-1). In some embodiments, the anti-Galectin-9 antibody promotes CD4+ cell activation by at least 30% (e.g., 31%, 35%, 40%, 50%, 60%, 70%, 80%, 90%, 95% or greater, including any increment therein). In a non-limiting example, the anti-Galectin antibody induces CD44 expression in CD4+ cells. In some embodiments, the anti-Galectin-9 antibody increases CD44 expression in CD4+ cells by at least 30% (e.g., 31%, 35%, 40%, 50%, 60%, 70%, 80%, 90%, 95% or greater, including any increment therein). In a non-limiting example, the anti-Galectin antibody induces IFNgamma expression in CD4+ cells. In some embodiments, the anti-Galectin-9 antibody increases IFNgamma expression in CD4+ cells by at least 30% (e.g., 31%, 35%, 40%, 50%, 60%, 70%, 80%, 90%, 95% or greater, including any increment therein). In a non-limiting example, the anti-Galectin antibody induces TNFalpha expression in CD4+ cells. In some embodiments, the anti-Galectin-9 antibody increases TNFalpha expression in CD4+ cells by at least 30% (e.g., 31%, 35%, 40%, 50%, 60%, 70%, 80%, 90%, 95% or greater, including any increment therein).

In some embodiments, the anti-Galectin-9 antibody promotes CD8+ cell activation by at least 30% (e.g., 31%, 35%, 40%, 50%, 60%, 70%, 80%, 90%, 95% or greater), including any increment therein). In a non-limiting example, the anti-Galectin antibody induces CD44 expression in CD8+ cells. In some embodiments, the anti-Galectin-9 antibody increases CD44 expression in CD8+ cells by at least 30% (e.g., 31%, 35%, 40%, 50%, 60%, 70%, 80%, 90%, 95% or greater, including any increment therein). In a non-limiting example, the anti-Galectin antibody induces IFNgamma expression in CD8+ cells. In some embodiments, the anti-Galectin-9 antibody increases IFNgamma expression in CD8+ cells by at least 30% (e.g., 31%, 35%, 40%, 50%, 60%, 70%, 80%, 90%, 95% or greater, including any increment therein). In a non-limiting example, the anti-Galectin antibody induces TNFalpha expression in CD8+ cells. In some embodiments, the anti-Galectin-9 antibody increases TNFalpha expression in CD8+ cells by at least 30% (e.g., 31%, 35%, 40%, 50%, 60%, 70%, 80%, 90%, 95% or greater, including any increment therein).

In some embodiments, an anti-Galectin-9 antibody as described herein has a suitable binding affinity for the target antigen (e.g., Galectin-9) or antigenic epitopes thereof. As used herein, “binding affinity” refers to the apparent association constant or KA. The KA is the reciprocal of the dissociation constant (K_{D}). The anti-Galectin-9 antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20).

These techniques can be used to measure the concentration of bound binding protein as a function of target protein concentration. Under certain conditions, the fractional concentration of bound binding protein ([Bound]/[Total]) is generally related to the concentration of total target protein ([Target]) by the following equation:

[Bound]/[Total]=[Target]/(Kd+[Target])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay. In some cases, the in vitro binding assay is indicative of in vivo activity. In other cases, the in vitro binding assay is not necessarily indicative of in vivo activity. In some cases, tight binding is beneficial, but in other cases tight binding is not as desirable in vivo, and an antibody with lower binding affinity is more desirable.

In some embodiments, the heavy chain of any of any of the anti-Galectin-9 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain) of any IgG subfamily as described herein.

In some embodiments, the heavy chain constant region of the antibodies described herein comprise a single domain (e.g., CH1, CH2, or CH3) or a combination of any of the single domains, of a constant region (e.g., SEQ ID NOs: 10, 12-14, and 21). In some embodiments, the light chain constant region of the antibodies described herein comprise a single domain (e.g., CL), of a constant region. Exemplary light and heavy chain sequences are listed below. Exemplary light and heavy chain sequences are listed below. The hIgG1 LALA sequence includes two mutations, L234A and L235A (EU numbering), which suppress FcgR binding as well as a P329G mutation (EU numbering) to abolish complement C1q binding, thus abolishing all immune effector functions. The hIgG4 Fab Arm Exchange Mutant sequence includes a mutation to suppress Fab Arm Exchange (S228P; EU numbering). An IL2 signal sequence (MYRMQLLSCIALSLALVTNS; SEQ ID NO: 9) can be located N-terminally of the variable region. It is used in expression vectors, which is cleaved during secretion and thus not in the mature antibody molecule. The mature protein (after secretion) starts with “EVQ” for the heavy chain and “DIM” for the light chain. Amino acid sequences of exemplary heavy chain constant regions are provided below:

hIgG1 Heavy Chain Constant Region
(SEQ ID NO: 10)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVELFPPKPKDTLMI
SRTPEVTCVVVDVSHEDPEVKENWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK*
hIgG1 LALA Heavy Chain Constant Region
(SEQ ID NO: 12)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKENWYVDGVEVHNAKTKPREEQYNSTYRVVSVLTVLHQDWLNGKEYKC
KVSNKALGAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK*
hIgG4 Heavy Chain Constant Region
(SEQ ID NO: 13)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPSCPAPEFLGGPSVELFPPKPKDTLMISRT
PEVTCVVVDVSQEDPEVQFNWYVDGVEVHNAKTKPREEQENSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSPGK*
hIgG4 Heavy Chain Constant Region
(SEQ ID NO: 20)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK*
hIgG4 mut Heavy Chain Constant Region
(SEQ ID NO: 14)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVELFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSPGK*
hIgG4 mut Heavy Chain Constant Region
(SEQ ID NO: 21)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK*

In some embodiments, anti-Galectin-9 antibodies having any of the above heavy chain constant regions are paired with a light chain having the following light chain constant region:

Light Chain Constant Region
(SEQ ID NO: 11)
TVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSENRGEC
Exemplary full-length anti-Galectin-9 antibodies are provided below:
G9.2-17 hIgG1 Heavy Chain
(SEQ ID NO: 16)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPKDTLMISRTPEVTCVVVD
VSHEDPEVKENWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK*
G9.2-17 hIgG1 LALA Heavy Chain
(SEQ ID NO: 17)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVELFPPKPKDTLMISRTPEVTCVVVD
VSHEDPEVKFNWYVDGVEVHNAKTKPREEQYNSTYRVVSVLTVLHQDWLNGKEYKCKVSNKALGAPIE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK*
G9.2-17 hIgG4 Heavy Chain
(SEQ ID NO: 18)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSE
FLYSRLTVDKSRWQEGNVFSCSVMHEALHNHYTQKSLSLSPGK*
G9.2-17 hIgG4 Heavy Chain
(SEQ ID NO: 22)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSE
FLYSRLTVDKSRWQEGNVESCSVMHEALHNHYTQKSLSLSLGK*
G9.2-17 hIgG4 Fab Arm Exchange mut Heavy Chain
(SEQ ID NO: 19)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVESCSVMHEALHNHYTQKSLSLSPGK*
G9.2-17 hIgG4 Fab Arm Exchange mut Heavy Chain
(SEQ ID NO: 23)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVELFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSE
FLYSRLTVDKSRWQEGNVFSCSVMHEALHNHYTQKSLSLSLGK*
Any of the above heavy chain can be paired with a Light Chain of
shown below:
(SEQ ID NO: 15)
DIQMTQSPSSLSASVGDRVTITCRASQSVSSAVAWYQQKPGKAPKLLIYSASSLYSGVPSRFSGSRSG
TDFTLTISSLQPEDFATYYCQQSSTDPITFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSENRGEC*

In some embodiments, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 10. In one embodiment, the constant region of the anti-Galectin-9 antibody comprises a heavy chain IgG1 constant region comprising SEQ ID NO: 13. In one embodiment, the constant region of the anti-Galectin-9 antibody comprises a heavy chain IgG4 constant region consisting of SEQ ID NO: 10.

In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 13. In one embodiment, the anti-Galectin-9 antibody comprises a heavy chain IgG4 constant region comprising SEQ ID NO: 13. In one embodiment, the anti-Galectin-9 antibody comprises a heavy chain IgG4 constant region consisting of SEQ ID NO: 13.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the anti-Galectin-9 antibody comprises a heavy chain IgG4 constant region comprising SEQ ID NO: 20. In one embodiment, the anti-Galectin-9 antibody comprises a heavy chain IgG4 constant region consisting of SEQ ID NO: 20.

In any of these embodiments, the anti-Galectin-9 antibody comprises a light chain constant region that has at least 80% (e.g., 80%, 85%, 90%, 91%, 92%, 93%, 94%, 95%, 96%, 97%, 98%, or 99% and any increment therein) sequence identity to SEQ ID NO: 11. In some embodiments, the anti-Galectin-9 antibody comprises a light chain constant region comprising SEQ ID NO: 11. In some embodiments, the anti-Galectin-9 antibody comprises a light chain constant region consisting of SEQ ID NO: 11.

In some embodiments, the IgG is a mutant with minimal Fc receptor engagement. In one example, the constant region is from a human IgG1 LALA. In one embodiment, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 12. In one embodiment, the anti-Galectin-9 antibody comprises a heavy chain IgG1 constant region comprising SEQ ID NO: 12. In one embodiment, the anti-Galectin-9 antibody comprises a heavy chain IgG1 constant region consisting of SEQ ID NO: 12.

In some embodiments, the anti-Galectin-9 antibody comprises a modified constant region. In some embodiments, the anti-Galectin-9 antibody comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8. In some embodiments, the IgG4 constant region is a mutant with reduced heavy chain exchange. In some embodiments, the constant region is from a human IgG4 Fab Arm Exchange mutant S228P.

In one embodiment, the constant region of the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 14. In one embodiment, the constant region of the anti-Galectin-9 antibody comprises a heavy chain IgG4 constant region comprising SEQ ID NO: 14. In one embodiment, the constant region of the anti-Galectin-9 antibody comprises a heavy chain IgG4 constant region consisting of SEQ ID NO: 14.

In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 21. In one embodiment, the anti-Galectin-9 antibody comprises a heavy chain IgG4 constant region comprising SEQ ID NO: 21. In one embodiment, the anti-Galectin-9 antibody comprises a heavy chain IgG4 constant region consisting of SEQ ID NO: 21.

In some embodiments, the anti-Galectin-9 antibody has chains corresponding to SEQ ID NO: 15 for the light chains; and the amino acid sequences of exemplary heavy chains correspond to SEQ ID NOs: 10 (hIgG1); 12 (hIgG1 LALA); 13 (hIgG4); 20 (hIgG4); 14 (hIgG4 mut); and 21 (hIgG4 mut).

In some embodiments, the anti-Galectin-9 antibody has a light chain comprising, consisting essentially of, or consisting of SEQ ID NO: 15. In some embodiments, the anti-Galectin-9 antibody has a heavy chain comprising, consisting essentially of, or consisting of any one of the sequences selected from the group consisting of SEQ ID NO: 16-19, 22 and 23. In some embodiments, the anti-Galectin-9 antibody has a light chain comprising, consisting essentially of, or consisting of SEQ ID NO: 15 and a heavy chain comprising, consisting essentially of, or consisting of any one of the sequences selected from the group consisting of SEQ ID NO: 16-19. In some embodiments, the anti-Galectin-9 antibody has a light chain comprising SEQ ID NO: 15 and a heavy chain comprising any one of the sequences selected from the group consisting of SEQ ID NO: 16-19, 22 and 23. In some embodiments, the anti-Galectin-9 antibody has a light chain consisting essentially of SEQ ID NO: 15 and a heavy chain consisting essentially of any one of the sequences selected from the group consisting of SEQ ID NO: 16-19, 22 and 23. In some embodiments, the anti-Galectin-9 antibody has a light chain consisting of SEQ ID NO: 15 and a heavy chain consisting of any one of the sequences selected from the group consisting of SEQ ID NO: 16-19, 22 and 23. In one specific embodiment, the anti-Galectin-9 antibody has a light chain consisting essentially of SEQ ID NO: 15 and a heavy chain consisting essentially of SEQ ID NO: 19. In another specific embodiment, the anti-Galectin-9 antibody has a light chain consisting essentially of SEQ ID NO: 15 and a heavy chain consisting essentially of SEQ ID NO: 20.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 16. In one embodiment, the anti-Galectin-9 antibody comprises a heavy chain sequence comprising SEQ ID NO: 16. In one embodiment, the anti-Galectin-9 antibody comprises a heavy chain sequence consisting of SEQ ID NO: 16.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 17. In one embodiment, the anti-Galectin-9 antibody comprises a heavy chain sequence comprising SEQ ID NO: 17. In one embodiment, the anti-Galectin-9 antibody comprises a heavy chain sequence consisting of SEQ ID NO: 17.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 18. In one embodiment, the anti-Galectin-9 antibody comprises a heavy chain sequence comprising SEQ ID NO: 18. In one embodiment, the anti-Galectin-9 antibody comprises a heavy chain sequence consisting of SEQ ID NO: 18.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 22. In one embodiment, the anti-Galectin-9 antibody comprises a heavy chain sequence comprising SEQ ID NO: 22. In one embodiment, the anti-Galectin-9 antibody comprises a heavy chain sequence consisting of SEQ ID NO: 22.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 19. In one embodiment, the anti-Galectin-9 antibody comprises a heavy chain sequence comprising SEQ ID NO: 19. In one embodiment, the anti-Galectin-9 antibody comprises a heavy chain sequence consisting of SEQ ID NO: 19.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 23. In one embodiment, the anti-Galectin-9 antibody comprises a heavy chain sequence comprising SEQ ID NO: 23. In one embodiment, the anti-Galectin-9 antibody comprises a heavy chain sequence consisting of SEQ ID NO: 23.

In any of these embodiments, the anti-Galectin-9 antibody comprises a light chain sequence having at least 80% (e.g., 80%, 85%, 90%, 91%, 92%, 93%, 94%, 95%, 96%, 97%, 98%, or 99% and any increment therein) sequence identity to SEQ ID NO: 15. In some embodiments, the anti-Galectin-9 antibody comprises a light chain sequence comprising SEQ ID NO: 15. In some embodiments, the anti-Galectin-9 antibody comprises a light chain sequence consisting of SEQ ID NO: 15.

In specific examples, the anti-Galectin-9 antibody used in the treatment methods disclosed herein has a heavy chain of SEQ ID NO:19 and a light chain of SEQ ID NO:15. In some embodiments, the anti-Galectin-9 antibody used in the treatment methods disclosed herein is G9.2-17 IgG4.

### Preparation of Anti-Galectin-9 Antibodies

Antibodies capable of binding Galectin-9 as described herein can be made by any method known in the art, including but not limited to, recombinant technology. One example is provided below.

Nucleic acids encoding the heavy and light chain of an anti-Galectin-9 antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-Galectin-9 antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-Galectin-9 antibody and the other encoding the light chain of the anti-Galectin-9 antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-Galectin-9 antibody as described herein, vectors (e.g., expression vectors) containing such; and host cells comprising the vectors are within the scope of the present disclosure.

Anti-Galectin-9 antibodies thus prepared can be characterized using methods known in the art, whereby reduction, amelioration, or neutralization of Galectin-9 biological activity is detected and/or measured. For example, in some embodiments, an ELISA-type assay is suitable for qualitative or quantitative measurement of Galectin-9 inhibition of Dectin-1 or TIM-3 signaling.

The bioactivity of an anti-Galectin-9 antibody can verified by incubating a candidate antibody with Dectin-1 and Galectin-9, and monitoring any one or more of the following characteristics: (a) binding between Dectin-1 and Galectin-9 and inhibition of the signaling transduction mediated by the binding; (b) preventing, ameliorating, or treating any aspect of a solid tumor; (c) blocking or decreasing Dectin-1 activation; (d) inhibiting (reducing) synthesis, production or release of Galectin-9. Alternatively, TIM-3 can be used to verify the bioactivity of an anti-Galectin-9 antibody using the protocol described above. Alternatively, CD206 can be used to verify the bioactivity of an anti-Galectin-9 antibody using the protocol described above.

In some embodiments, bioactivity or efficacy is assessed in a subject, e.g., by measuring peripheral and intra-tumoral T cell ratios, T cell activation, or by macrophage phenotyping.

Additional assays to determine bioactivity of an anti-Galectin-9 antibody include measurement of CD8+ and CD4+(conventional) T-cell activation (in an in vitro or in vivo assay, e.g., by measuring inflammatory cytokine levels, e.g., IFNgamma, TNFalpha, CD44, ICOS granzyme B, Perforin, IL2 (upregulation); CD26L and IL-10 (downregulation)); measurement of reprogramming of macrophages (in vitro or in vivo), e.g., from the M2 to the M1 phenotype (e.g., increased MHCII, reduced CD206, increased TNF-alpha and iNOS), Alternatively, levels of ADCC can be assessed, e.g., in an in vitro assay, as described herein.

### Pharmaceutical Compositions

The anti-Galectin-9 antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Areiams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Areiams and Wilkins, Ed. K. E. Hoover). Acceptable carriers, excipients, or stabilizers are nontoxic to recipients at the dosages and concentrations used, and comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, the anti-Galectin-9 antibodies, or the encoding nucleic acid(s), are be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Suitable surface-active agents (surfactant) include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent are conveniently comprise between 0.05 and 5% surface-active agent, and can be between 0.1 and 2.5%. It are be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It are be appreciated that other ingredients may be added, for example glycerol or glucose, to adjust the tonicity of the emulsion. Suitable emulsions are typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Combined Cancer Therapy

The present disclosure provides methods for treating solid tumors, such as PDAC, colorectal cancer (CRC), hepatocellular carcinoma (HCC), or cholangiocarcinoma (CAA), using any of the anti-Galectin antibodies, for example G9.2-17 (e.g., G9.2-17(IgG4)), in combination with one or more chemotherapeutics such as gemcitabine and/or paclitaxel (e.g., Abraxane®).

Without being bound by theory, it is thought that anti-Galectin-9 antibodies, through their inhibition of Dectin-1, can reprogram immune responses against tumor cells via, e.g., inhibiting the activity of γδ T cells infiltrated into tumor microenvironment, and/or enhancing immune surveillance against tumor cells by, e.g., activating CD4+ and/or CD8+ T cells. Thus, combined use of an anti-Galectin-9 antibody and one or more chemotherapeutics such as those described herein would be expected to significantly enhance anti-tumor efficacy.

Pancreatic ductal adenocarcinoma (PDA) is a devastating disease with few long-term survivors (Yadav et al., Gastroenterology, 2013, 144, 1252-1261). Inflammation is paramount in PDA progression as oncogenic mutations alone, in the absence of concomitant inflammation, are insufficient for tumorigenesis (Guerra et al., Cancer Cell, 2007, 11, 291-302). Innate and adaptive immunity cooperate to promote tumor progression in PDA. In particular, specific innate immune subsets within the tumor microenvironment (TME) are apt at educating adaptive immune effector cells towards a tumor-permissive phenotype. Antigen presenting cell (APC) populations, including M2-polarized tumor-associated macrophages (TAMs) and myeloid dendritic cells (DC), induce the generation of immune suppressive Th2 cells in favor of tumor-protective Th1 cells (Ochi et al., J of Exp Med., 2012, 209, 1671-1687; Zhu et al., Cancer Res., 2014, 74, 5057-5069). Similarly, it has been shown that myeloid derived suppressor cells (MDSC) negate anti-tumor CD8⁺ cytotoxic T-Lymphocyte (CTL) responses in PDA and promote metastatic progression (Connolly et al., J Leuk Biol., 2010, 87, 713-725; Pylayeva-Gupta et al., Cancer Cell, 2012, 21, 836-847; Bayne et al., Cancer Cell, 2012, 21, 822-835).

Colorectal cancer (CRC), also known as bowel cancer, colon cancer, or rectal cancer, is any cancer affecting the colon and the rectum. CRC is known to be driven by genetic alterations of tumor cells and is also influenced by tumor-host interactions. Recent reports have demonstrated a direct correlation between the densities of certain T lymphocyte subpopulations and a favorable clinical outcome in CRC, supporting a major role of T-cell-mediated immunity in repressing tumor progression of CRC.

Hepatocellular carcinoma (HCC) is the most common type of primary liver cancer. Hepatocellular carcinoma occurs most often in people with chronic liver diseases, such as cirrhosis caused by hepatitis B or hepatitis C infection. HCC is usually accompanied by cirrhotic liver with extensive lymphocyte infiltration due to chronic viral infection. Many studies have demonstrated that tumor-infiltrating effector CD8+ T cells and T helper 17 (Th17) cells correlate with improved survival after surgical resection of tumors. However, tumor-infiltrating effector T cells fail to control tumor growth and metastasis (Pang et al., Cancer Immunol Immunother 2009; 58:877-886).

Cholangiocarcinoma is a group of cancers that begin in the bile ducts. Cholangiocarcinoma is commonly classified by its location in relation to the liver. For example, intrahepatic cholangiocarcinoma, accounting for less than 10% of all cholangiocarcinoma cases, begins in the small bile ducts within the liver. In another example, perihilar cholangiocarcinoma (also known as a Klatskin tumor), accounting for more than half of the cholangiocarcinoma cases, begins in hilum, where two major bile ducts join and leave the liver. Others are classified as distal cholangiocarcinomas, which begin in bile ducts outside the liver.

In some aspects, the present disclosure provides methods of treating a solid tumor such as those disclosed herein. In some embodiments, the present disclosure provides methods for reducing, ameliorating, or eliminating one or more symptom(s) associated with the solid tumor. The treatment methods disclosed herein involve the combined therapy of an anti-Gal9 antibody such as G9.2-17 and one or more chemotherapeutics. In some examples, an effective amount of the anti-Gal9 antibody is given to a subject having a solid tumor (e.g., PDAC), wherein the subject is on a treatment involving the one or more chemotherapeutics. In some examples, an effective amount of the one or more chemotherapeutics are given to a subject having a solid tumor (e.g., PDAC), wherein the subject is on a treatment involving the anti-Gal9 antibody. In other examples, an effective amount of the anti-Gal9 antibody and an effective amount of the one or more chemotherapeutics are given to the subject, concurrently or sequentially.

In some embodiments, the methods of the present disclosure increase anti-tumor activity (e.g., reduce cell proliferation, tumor growth, tumor volume, and/or tumor burden or load or reduce the number of metastatic lesions over time) by at least about 10%, 20%, 25%, 30%, 40%, 50%, 60%, 70%, 75%, 80%, 85%, 90%, 95%, or more as compared to levels prior to treatment or in a control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more. In some embodiments, before, during, and after the administration of the pharmaceutical composition, cancerous cells and/or biomarkers in a subject are measured in a biological sample, such as blood, serum, plasma, urine, peritoneal fluid, and/or a biopsy from a tissue or organ. In some embodiments, the methods include administration of the compositions of the invention to reduce tumor volume, size, load or burden in a subject to an undetectable size, or to less than about 1%, 2%, 5%, 10%, 20%, 25%, 30%, 40%, 50%, 60%, 70%, 75%, 80%, or 90% of the subject's tumor volume, size, load or burden prior to treatment. In other embodiments, the methods include administration of the compositions of the invention to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment. In other embodiments, the methods include administration of the compositions of the invention to reduce the development of or the number or size of metastatic lesions in a subject to an undetectable rate, or to less than about 1%, 2%, 5%, 10%, 20%, 25%, 30%, 40%, 50%, 60%, 70%, 75%, 80%, or 90% of the rate prior to treatment.

The term “about” or “approximately” means within an acceptable error range for the particular value as determined by one of ordinary skill in the art, which are depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, the antibodies described herein, e.g., G9.2-17 such as its IgG4 form, are administered to a subject in need of the treatment at an amount sufficient to inhibit the activity of Galectin-9 (and/or Dectin-1 or TIM-3 or CD206) in immune suppressive immune cells in a tumor by at least 20% (e.g., 30%, 40%, 50%, 60%, 70%, 80%, 90% or greater) in vivo. In other embodiments, the antibodies described herein, e.g., G9.2-17, are administered in an amount effective in reducing the activity level of Galectin-9 (and/or Dectin-1 or TIM-3 or CD206) in immune suppressive immune cells in a tumor by at least 20% (e.g., 30%, 40%, 50%, 60%, 70%, 80%, 90% or greater) (as compared to levels prior to treatment or in a control subject). In some embodiments, the antibodies described herein, e.g., G9.2-17, are administered to a subject in need of the treatment at an amount sufficient to promote M1-like programming in TAMs by at least 20% (e.g., 30%, 40%, 50%, 60%, 70%, 80%, 90% or greater) in vivo (as compared to levels prior to treatment or in a control subject).

Conventional methods, known to those of ordinary skill in the art of medicine, can be used to administer the pharmaceutical composition to the subject, depending upon the type of disease to be treated or the site of the disease. In some embodiments, the anti-Galectin-9 antibody can be administered to a subject by intravenous infusion.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, the methods are provided, the anti-Galectin-9 antibody is administered concurrently with the one or more chemotherapeutics. In some embodiments, the anti-Galectin-9 antibody is administered before or after the one or more chemotherapeutics. In some embodiments, the one or more chemotherapeutics are administered systemically. In some embodiments, the one or more chemotherapeutics is administered locally. In some embodiments, the one or more chemotherapeutics is administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In one embodiment, the one or more chemotherapeutics is administered to the subject by intravenous infusion.

An effective amount of the pharmaceutical composition described herein can be administered to a subject (e.g., a human) in need of the treatment via a suitable route, systemically or locally. In some embodiments, the anti-galectin-9 antibodies are administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In one embodiment, the anti-galectin-9 antibody is administered to the subject by intravenous infusion.

As used herein, “an effective amount” refers to the amount of each active agent required to confer therapeutic effect on the subject, either alone or in combination with one or more other active agents. In some embodiments, the therapeutic effect is reduced Galectin-9 activity and/or amount/expression, reduced Dectin-1 signaling, reduced TIM-3 signaling, reduced CD206 signaling, or increased anti-tumor immune responses in the tumor microenvironment. Non-limiting examples of increased anti-tumor responses include increased activation levels of effector T cells, or switching of the TAMs from the M2 to the M1 phenotype. In some cases, the anti-tumor response includes increased ADCC responses. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally contribute to the determination of the dosage. For example, antibodies that are compatible with the human immune system, such as humanized antibodies or fully human antibodies, are in some instances used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein are determined empirically in individuals who have been given one or more administration(s) of the antibody. Individuals are given incremental dosages of the antagonist. To assess efficacy of the antagonist, an indicator of the disease/disorder can be followed.

Any of the anti-Galectin-9 antibodies described herein can be used in any of the methods described herein. In some embodiments, the anti-Galectin-9 antibody is G9.2-17. The G9.2-17 antibody may be an IgG4 molecule (G9.2-17(IgG4) as disclosed herein. In specific examples, the anti-Galectin-9 antibody (G9.2-17) used herein has a heavy chain of SEQ ID NO:19 and a light chain of SEQ ID NO:15. The anti-Gal9 antibody may be formulated as disclosed herein and given to a subject in need of the treatment via a suitable route, for example, intravenous infusion.

In some instances, the anti-Galectin-9 antibody as disclosed herein (e.g., G9.2-17) can be administered to a subject at a suitable dose, for example, about 0.5 to about 32 mg/kg. Examples include 0.5 mg/kg to 1 mg/kg, 1 mg/kg to 2 mg/kg, 2 mg/kg to 3 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges. In some embodiments, the antibody is administered at a dose of about 0.5 about mg/kg to 1 mg/kg, about 1 mg/kg to 2 mg/kg, about 2 mg/kg to 4 mg/kg, about 4 mg/kg to 8 mg/kg, about 8 mg/kg to 12 mg/kg, about 12 mg/kg to 16 mg/kg, about 16 mg/kg to 20 mg/kg, about 20 mg/kg to 24 mg/kg, about 24 mg/kg to 28 mg/kg, or about 28 mg/kg to 32 mg/kg (e.g., about 0.5 mg/kg, about 1 mg/kg, about 2 mg/kg, about 3 mg/kg, about 4 mg/kg, about 5 mg/kg, about 6 mg/kg, 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) or any incremental doses within these ranges.

In some embodiments, the Galectin-9 antibody is administered at 2 mg/kg. In some embodiments, the Galectin-9 antibody is administered at 4 mg/kg. In some embodiments, the Galectin-9 antibody is administered at 8 mg/kg. In some embodiments, the Galectin-9 antibody is administered at 12 mg/kg. In some embodiments, the Galectin-9 antibody is administered at 16 mg/kg. In some instances, multiple doses of the anti-Galectin-9 antibody can be administered to a subject at a suitable interval or cycle, for example, once every week, once every two to four weeks (e.g., every two, three, or four weeks). The treatment may last for a suitable period, for example, up to 3 months, up to 6 months, or up to 12 months or up to 24 months or longer.

In some examples, the anti-Galectin-9 antibody is administered to a human patient having a solid tumor as disclosed herein (e.g., PDA) at a dose of about 3 mg/kg once every two weeks via intravenous infusion. In other examples, the anti-Galectin-9 antibody is administered to the human patient having the target solid tumor at a dose of about 15 mg/kg once every two weeks via intravenous infusion.

In some examples, about 2 mg/kg to 16 mg/kg anti-Gal9 antibody (e.g., G9.2-17 in IgG4 form) may be given to a subject in need of the treatment once every two weeks. In some examples, the anti-Gal9 antibody (e.g., G9.2-17 in IgG4 form as disclosed herein, having a heavy chain of SEQ ID NO: 19 and a light chain of SEQ ID NO: 15) is administered to the subject at a dose of about 0.5 mg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg or any increment therein, once every two weeks by intravenous injection.

In some examples, the anti-Gal9 antibody (e.g., G9.2-17 in IgG4 form as disclosed herein, having a heavy chain of SEQ ID NO: 19 and a light chain of SEQ ID NO: 15) is administered to the subject at a dose of about 2 mg/kg once every two weeks by intravenous injection. In some examples, the anti-Gal9 antibody is administered to the subject at a dose of about 4 mg/kg once every two weeks by intravenous injection. In some examples, the anti-Gal9 antibody is administered to the subject at a dose of about 8 mg/kg once every two weeks by intravenous injection. In some examples, the anti-Gal9 antibody is administered to the subject at a dose of about 12 mg/kg once every two weeks by intravenous injection. In some examples, the anti-Gal9 antibody is administered to the subject at a dose of about 16 mg/kg once every two weeks by intravenous injection.

In some examples, the anti-Gal9 antibody (e.g., G9.2-17 in IgG4 form as disclosed herein, having a heavy chain of SEQ ID NO: 19 and a light chain of SEQ ID NO: 15) is administered to the subject at a dose of 0.5 mg/kg to 1 mg/kg, about 1 mg/kg to 2 mg/kg, about 3 mg/kg to 4 mg/kg, about 4 mg/kg to 8 mg/kg, about 8 mg/kg to 12 mg/kg, about 12 mg/kg to 16 mg/kg, about 16 mg/kg to 20 mg/kg, about 20 mg/kg to 24 mg/kg, about 24 mg/kg to 28 mg/kg, or about 28 mg/kg to 32 mg/kg (e.g., about 0.5 mg/kg, about 1 mg/kg, about 2 mg/kg, about 3 mg/kg, about 4 mg/kg, about 5 mg/kg, about 6 mg/kg, 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) any increment therein, once a week by intravenous injection.

In some examples, the anti-Gal9 antibody (e.g., G9.2-17 in IgG4 form as disclosed herein, having a heavy chain of SEQ ID NO: 19 and a light chain of SEQ ID NO: 15) is administered to the subject at a dose of 0.5 mg/kg to 1 mg/kg, 1 mg/kg to 2 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges or any incremental doses within these ranges, once a week by intravenous injection.

In some examples, the anti-Gal9 antibody (e.g., G9.2-17 in IgG4 form as disclosed herein, having a heavy chain of SEQ ID NO: 19 and a light chain of SEQ ID NO: 15) is administered to the subject at a dose of about 0.5 mg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg or any increment therein, once a week by intravenous injection.

In specific embodiments, the interval or cycle is 1 week. In specific embodiments, the interval or cycle is 2 weeks. In some embodiments, the regimen is once every 2 weeks for one cycle, once every 2 weeks for two cycles, once every 2 weeks for three cycles, once every 2 weeks for four cycles, or once every 2 weeks for more than four cycles. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

In specific embodiments, the interval or cycle is 3 weeks. In some embodiments, the regimen is once every 3 weeks for one cycle, once every 3 weeks for two cycles, once every 3 weeks for three cycles, once every 3 weeks for four cycles, or once every 3 weeks for more than four cycles. In some embodiments, the treatment is once every 3 weeks for 1 to 3 months, once every 3 weeks for 3 to 6 months, once every 3 weeks for 6 to 12 months, or once every 3 weeks for 12 to 24 months, or longer.

In specific embodiments, the interval or cycle is 4 or more weeks. In some embodiments, the regimen is once every 4 or more weeks for one cycle, once every 4 or more weeks for two cycles, once every 4 or more weeks for three cycles, once every 4 or more weeks for four cycles, or once every 4 or more weeks for more than four cycles. In some embodiments, the treatment is once every 4 or more weeks for 1 to 3 months, once every 4 or more weeks for 3 to 6 months, once every 4 or more weeks for 6 to 12 months, or once every 4 or more weeks for 12 to 24 months, or longer. In some embodiments, the treatment is a combination of treatment at various time, e.g., a combination or 2 weeks, 3 weeks, 4 or more 4 weeks. In some embodiments, the treatment interval is adjusted in accordance with the patient's response to treatment. In some embodiments, the dosage(s) is adjusted in accordance with the patient's response to treatment. In some embodiments, the dosages are altered between treatment intervals. In some embodiments, the treatment may be temporarily stopped. In some embodiments, anti-Galectin-9 therapy is temporarily stopped. In some embodiments, chemotherapy is temporarily stopped. In some embodiments, both are temporarily stopped. In any of these embodiments, the anti-Gal9 antibody may be G9.2-17 in IgG4 form as disclosed herein, having a heavy chain of SEQ ID NO: 19 and a light chain of SEQ ID NO: 15).

The one or more chemotherapeutics may comprise an antimetabolite, a microtubule inhibitor, or a combination thereof. Antimetabolites include, for example, folic acid antagonist (e.g., methotrexate) and nucleotide analogs such as pyrimidine antagonist (e.g., 5-fluorouracil, foxuridine, cytarabine, capecitabine, and gemcitabine), purine antagonist (e.g., 6-mercaptopurine and 6-thioguanine), and adenosine deaminase inhibitor (e.g., cladribine, fludarabine and pentostatin).

In some examples, the antimetabolites used in the methods disclosed herein is gemcitabine, which may be given by intravenous infusion. The amount of gemcitabine to be given to a subject depends on many factors, including height and weight, general health or other health problems, and the type of cancer to be treated, which would be within the knowledge of a medical practitioner following guidance provided by the Food and Drug Administration (e.g., see the drug labels of approved gemcitabine products). In some examples, a subject may be administered gemcitabine by intravenous infusion at a dose of 1000 mg/m² optionally over 30 minutes once weekly for up to 7 weeks, followed by one week rest from the treatment. Subsequent cycles may consist of infusion once weekly for three consecutive weeks out of every four weeks. If one or more adverse effects occur, the dose of gemcitabine may be reduced or the treatment may be withheld. More details for managing adverse effects associated with gemcitabine treatment are provided in Example 2 below.

Microtubule inhibitors are a class of compounds that inhibit the formation of cellular microtubules, thereby blocking cell proliferation. In some examples, the microtubule inhibitor is a stabilizing agent that promotes polymerization of microtubules. Examples include taxanes and epothilones. In other examples, the microtubule inhibitor is a destabilizing agent that promotes depolymerization of microtubules. Examples include vinca alkaloids. In some examples, the microtubule inhibitor used in the methods disclosed herein is paclitaxel. In some instances, the paclitaxel is in free form. In other instances, the paclitaxel is conjugated to a protein, for example, albumin. In specific examples, the paclitaxel is Abraxane®, which is nanoparticle albumin-conjugated paclitaxel.

The amount of paclitaxel, e.g., protein-bound paclitaxel such as nab-paclitaxel, to be given to a subject depends on many factors, including height and weight, general health or other health problems, and the type of cancer to be treated, which would be within the knowledge of a medical practitioner following guidance provided by the Food and Drug Administration (e.g., see the drug labels of approved paclitaxel products). For example, when nanoparticle albumin-conjugated paclitaxel (nab-paclitaxel, e.g., Abraxane®), it can be given to a subject by intravenous injection at 260 mg/m² over 30 minutes every 3 weeks. The dose of paclitaxel may be reduced if severe adverse effects (e.g., neutropenia or severe sensory neuropathy) are observed. In some instances, the dose of nab-paclitaxel may be reduced to 180 mg/m². When in combination with the anti-Gal9 antibody, the dose of paclitaxel may be 125 mg/m². If needed, the dose of paclitaxel may be reduced to 100 mg/m² or 75 mg/m². More details for managing side effects associated with paclitaxel are provided in Example 2 below.

In some specific examples, the anti-Gal9 antibody (e.g., G9.2-17 in IgG4 form), gemcitabine, and paclitaxel (e.g., nanoparticle albumin-conjugated paclitaxel or Abraxane®) may be administered to a subject in need of the treatment following the treatment regimen and dosing schedules provided in Example 2 below. For example, the treatment may comprise one or more cycles, each consisting of 28 days. In each cycle, the anti-Gal9 antibody (e.g., G9.2-17(IgG4)) is given to the subject (e.g., a human patient having PDAC) once every two weeks (e.g., on Day 1 and Day 15) at a dose of about 2 mg/kg to 16 mg/mg (e.g., about 2 mg/kg, about 4 mg/kg, about 8 mg/kg, about 12 mg/kg, or about 16 mg/kg) via intravenous infusion. Gemcitabine and paclitaxel (e.g., protein-bound paclitaxel such as Abraxane®) can be administered to the subject once every week for three weeks followed by one week without treatment (e.g., on Day 1, Day 8, and Day 15 in the 28-day cycle), using the dosage and dosing scheduled as approved by the FDA. For example, gemcitabine may be given to the subject once every week at 1000 mg/m² in each cycle via intravenous injection and paclitaxel may be given to the subject once every week at 125 mg/m². When needed, the dose of gemcitabine may be reduced to 800 mg/m² or 600 mg/m². Alternatively or in addition, the dose of paclitaxel may be reduced to 100 mg/m² or 75 mg/m².

In some embodiments, the method for treating a solid tumor (e.g., PDA) described herein comprises one or more treatment cycle(s) of 28 days, wherein the anti-Gal9 antibody is administered to the subject on day 1 and day 15 (i.e, once every 2 weeks (q2w)) at a dose of about 0.5 mg/kg to about 32 mg/kg via intravenous infusion and gemcitabine and paclitaxel (e.g., nanoparticle albumin-bound paclitaxel) are administered to the subject on day 1, day 8, and day 15. In some examples, paclitaxel is administered to the subject at 125 mg/m² intravenously (e.g., intravenous injection). In some examples, gemcitabine is administered to the subject at 1000 mg/m² intravenously (e.g., intravenous injection). When needed, the dose of gemcitabine may be reduced to 800 mg/m² or 600 mg/m². Alternatively or in addition, the dose of paclitaxel may be reduced to 100 mg/m² or 75 mg/m².

In some embodiments, the method for treating a solid tumor (e.g., PDA) described herein comprises one or more treatment cycle(s) of 28 days, wherein the anti-Gal9 antibody is administered to the subject on day 1 and day 15 (i.e, once every 2 weeks (q2w)) at a dose of include about 0.5 about mg/kg to 1 mg/kg, about 1 mg/kg to 2 mg/kg, about 3 mg/kg to 4 mg/kg, about 4 mg/kg to 8 mg/kg, about 8 mg/kg to 12 mg/kg, about 12 mg/kg to 16 mg/kg, about 16 mg/kg to 20 mg/kg, about 20 mg/kg to 24 mg/kg, about 24 mg/kg to 28 mg/kg, or about 28 mg/kg to 32 mg/kg (e.g., about 0.5 mg/kg, about 1 mg/kg, about 2 mg/kg, about 3 mg/kg, about 4 mg/kg, about 5 mg/kg, about 6 mg/kg, 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) via intravenous infusion and gemcitabine and paclitaxel (e.g., nanoparticle albumin-bound paclitaxel) are administered to the subject on day 1, day 8, and day 15. In some examples, paclitaxel is administered to the subject at 125 mg/m² intravenously (e.g., intravenous injection). In some examples, gemcitabine is administered to the subject at 1000 mg/m² intravenously (e.g., intravenous injection). When needed, the dose of gemcitabine may be reduced to 800 mg/m² or 600 mg/m². Alternatively or in addition, the dose of paclitaxel may be reduced to 100 mg/m² or 75 mg/m².

In some embodiments, the method for treating a solid tumor (e.g., PDA) described herein comprises one or more treatment cycle(s) of 28 days, wherein the anti-Gal9 antibody is administered to the subject on day 1 and day 15 (i.e., once every 2 weeks (q2w)) at a dose of include 0.5 mg/kg to 1 mg/kg, 1 mg/kg to 2 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges via intravenous infusion and gemcitabine and paclitaxel (e.g., nanoparticle albumin-bound paclitaxel) are administered to the subject on day 1, day 8, and day 15. In some examples, paclitaxel is administered to the subject at 125 mg/m² intravenously (e.g., intravenous injection). In some examples, gemcitabine is administered to the subject at 1000 mg/m² intravenously (e.g., intravenous injection). When needed, the dose of gemcitabine may be reduced to 800 mg/m² or 600 mg/m². Alternatively or in addition, the dose of paclitaxel may be reduced to 100 mg/m² or 75 mg/m².

In some embodiments, the method for treating a solid tumor (e.g., PDA) described herein comprises one or more treatment cycle(s) of 28 days, wherein the anti-Gal9 antibody is administered to the subject on day 1 and day 15 (i.e, once every 2 weeks (q2w)) at a dose of about 2 mg/kg to about 16 mg/kg via intravenous infusion and gemcitabine and paclitaxel (e.g., nanoparticle albumin-bound paclitaxel) are administered to the subject on day 1, day 8, and day 15. In some examples, paclitaxel is administered to the subject at 125 mg/m² intravenously (e.g., intravenous injection). In some examples, gemcitabine is administered to the subject at 1000 mg/m² intravenously (e.g., intravenous injection). When needed, the dose of gemcitabine may be reduced to 800 mg/m² or 600 mg/m². Alternatively or in addition, the dose of paclitaxel may be reduced to 100 mg/m² or 75 mg/m².

In some embodiments, the method for treating a solid tumor (e.g., PDA) described herein comprises one or more treatment cycle(s) of 28 days, wherein the anti-Gal9 antibody is administered to the subject on day 1 and day 15 (i.e, once every 2 weeks (q2w)) at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg or any increment therein, via intravenous infusion and gemcitabine and paclitaxel (e.g., nanoparticle albumin-bound paclitaxel) are administered to the subject on day 1, day 8, and day 15. In some examples, paclitaxel is administered to the subject at 125 mg/m² intravenously (e.g., intravenous injection). In some examples, gemcitabine is administered to the subject at 1000 mg/m² intravenously (e.g., intravenous injection). When needed, the dose of gemcitabine may be reduced to 800 mg/m² or 600 mg/m². Alternatively or in addition, the dose of paclitaxel (e.g., nanoparticle albumin-bound paclitaxel) may be reduced to 100 mg/m² or 75 mg/m².

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 0.5 mg/kg to 1 mg/kg, about 1 mg/kg to 2 mg/kg, about 3 mg/kg to 4 mg/kg, about 4 mg/kg to 8 mg/kg, about 8 mg/kg to 12 mg/kg, about 12 mg/kg to 16 mg/kg, about 16 mg/kg to 20 mg/kg, about 20 mg/kg to 24 mg/kg, about 24 mg/kg to 28 mg/kg, or about 28 mg/kg to 32 mg/kg (e.g., about 0.5 mg/kg, about 1 mg/kg, about 2 mg/kg, about 3 mg/kg, about 4 mg/kg, about 5 mg/kg, about 6 mg/kg, 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) any increment therein, via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of 0.5 mg/kg to 1 mg/kg, 1 mg/kg to 2 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any increment therein, via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) or any increment therein, via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg or any increment therein, via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 2 mg/kg via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 4 mg/kg via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 8 mg/kg via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more cycle(s) treatment of 28 days, wherein
(1) anti-Gal9 antibody is administered to the subject on day 1 and day 15 (i.e, once every 2 weeks (q2w)) at a dose of about 12 mg/kg via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 16 mg/kg via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 32 mg/kg via intravenous infusion,
(2) gemcitabine is administered to the subject on day 1, day 8, and day 15 at a dose of 1000 mg/m² intravenously (e.g., intravenous injection),
(3) paclitaxel (e.g., nanoparticle albumin-bound paclitaxel) is administered to the subject on day 1, day 8, and day 15 at a dose of 125 mg/m² intravenously (e.g., intravenous injection).

In any of the above administration method embodiments, when needed, the dose of gemcitabine may be reduced to 800 mg/m² or 600 mg/m², and alternatively or in addition, the dose of paclitaxel (e.g., nanoparticle albumin-bound paclitaxel) may be reduced to 100 mg/m² or 75 mg/m².

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 0.5 mg/kg to 1 mg/kg, about 1 mg/kg to 2 mg/kg, about 3 mg/kg to 4 mg/kg, about 4 mg/kg to 8 mg/kg, about 8 mg/kg to 12 mg/kg, about 12 mg/kg to 16 mg/kg, about 16 mg/kg to 20 mg/kg, about 20 mg/kg to 24 mg/kg, about 24 mg/kg to 28 mg/kg, or about 28 mg/kg to 32 mg/kg (e.g., about 0.5 mg/kg, about 1 mg/kg, about 2 mg/kg, about 3 mg/kg, about 4 mg/kg, about 5 mg/kg, about 6 mg/kg, 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) or any increment therein, via intravenous infusion,
(2) gemcitabine is administered to the subject on day 1, day 8, and day 15 at a dose of 800 mg/m², 600 mg/m², or 1000 mg/m² intravenously (e.g., intravenous injection),
(3) paclitaxel (e.g., nanoparticle albumin-bound paclitaxel) is administered to the subject on day 1, day 8, and day 15 at a dose of 100 mg/m², 75 mg/m² or 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of 0.5 mg/kg to 1 mg/kg, 1 mg/kg to 2 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges or any incremental doses within these ranges, or any increment therein, via intravenous infusion,
(2) gemcitabine is administered to the subject on day 1, day 8, and day 15 at a dose of 800 mg/m², 600 mg/m², or 1000 mg/m² intravenously (e.g., intravenous injection),
(3) paclitaxel (e.g., nanoparticle albumin-bound paclitaxel) is administered to the subject on day 1, day 8, and day 15 at a dose of 100 mg/m², 75 mg/m² or 125 mg/m² intravenously (e.g., intravenous injection).

In some embodiments, the method comprises one or more treatment cycle(s) of 28 days, wherein
(1) anti-Gal9 antibody is administered to the subject on day 1 and day 15 (i.e, once every 2 weeks (q2w)) at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg or any increment therein, via intravenous infusion,
(2) gemcitabine is administered to the subject on day 1, day 8, and day 15 at a dose of 800 mg/m², 600 mg/m², or 1000 mg/m² intravenously (e.g., intravenous injection),
(3) paclitaxel (e.g., nanoparticle albumin-bound paclitaxel) is administered to the subject on day 1, day 8, and day 15 at a dose of 100 mg/m², 75 mg/m² or 125 mg/m² intravenously (e.g., intravenous injection).

In any of the above administration methods, treatment cycles may continue over a period of 12-24 months.

In any of the method embodiments described herein, the anti-galectin-9 antibody can be administered (alone or in combination with one or more chemotherapeutic agents, e.g., gemicitabine and nab-paclitaxel, e.g., at the doses described herein) once a week, once every 2 weeks for one cycle, once every 2 weeks for two cycles, once every 2 weeks for three cycles, once every 2 weeks for four cycles, or once every 2 weeks for more than four cycles. In some embodiments, the treatment is 1 to 3 months, 3 to 6 months, 6 to 12 months, 12 to 24 months, or longer. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

In some embodiments, the method for treating a solid tumor (e.g., PDA) described herein comprises one or more treatment cycle(s) of 28 days, wherein the anti-Gal9 antibody is administered to the subject on day 1, day 7, day 15, and day 21 (i.e, once weekly (q1w)) at a dose of about 0.5 mg/kg to 1 mg/kg, about 1 mg/kg to 2 mg/kg, about 3 mg/kg to 4 mg/kg, about 4 mg/kg to 8 mg/kg, about 8 mg/kg to 12 mg/kg, about 12 mg/kg to 16 mg/kg, about 16 mg/kg to 20 mg/kg, about 20 mg/kg to 24 mg/kg, about 24 mg/kg to 28 mg/kg, or about 28 mg/kg to 32 mg/kg (e.g., about 0.5 mg/kg, about 1 mg/kg, about 2 mg/kg, about 3 mg/kg, about 4 mg/kg, about 5 mg/kg, about 6 mg/kg, 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) or any increment therein, via intravenous infusion and gemcitabine and paclitaxel (e.g., nanoparticle albumin-bound paclitaxel) are administered to the subject on day 1, day 8, and day 15. In some examples, paclitaxel is administered to the subject at 125 mg/m² intravenously (e.g., intravenous injection). In some examples, gemcitabine is administered to the subject at 1000 mg/m² intravenously (e.g., intravenous injection). When needed, the dose of gemcitabine may be reduced to 800 mg/m² or 600 mg/m². Alternatively or in addition, the dose of paclitaxel (e.g., nanoparticle albumin-bound paclitaxel) may be reduced to 100 mg/m² or 75 mg/m².

In some embodiments, the method for treating a solid tumor (e.g., PDA) described herein comprises one or more treatment cycle(s) of 28 days, wherein the anti-Gal9 antibody is administered to the subject on day 1, day 7, day 15, and day 21 (i.e, once weekly (q1w)) at a dose of 0.5 mg/kg to 1 mg/kg, 1 mg/kg to 2 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges or any incremental doses within these ranges, via intravenous infusion and gemcitabine and paclitaxel (e.g., nanoparticle albumin-bound paclitaxel) are administered to the subject on day 1, day 8, and day 15. In some examples, paclitaxel is administered to the subject at 125 mg/m² intravenously (e.g., intravenous injection). In some examples, gemcitabine is administered to the subject at 1000 mg/m² intravenously (e.g., intravenous injection). When needed, the dose of gemcitabine may be reduced to 800 mg/m² or 600 mg/m². Alternatively or in addition, the dose of paclitaxel (e.g., nanoparticle albumin-bound paclitaxel) may be reduced to 100 mg/m² or 75 mg/m².

In some embodiments, the method for treating a solid tumor (e.g., PDA) described herein comprises one or more treatment cycle(s) of 28 days, wherein the anti-Gal9 antibody is administered to the subject on day 1, day 7, day 15, and day 21 (i.e, once weekly (q1w)) at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg or any increment therein, via intravenous infusion and gemcitabine and paclitaxel (e.g., nanoparticle albumin-bound paclitaxel) are administered to the subject on day 1, day 8, and day 15. In some examples, paclitaxel is administered to the subject at 125 mg/m² intravenously (e.g., intravenous injection). In some examples, gemcitabine is administered to the subject at 1000 mg/m² intravenously (e.g., intravenous injection). When needed, the dose of gemcitabine may be reduced to 800 mg/m² or 600 mg/m². Alternatively or in addition, the dose of paclitaxel (e.g., nanoparticle albumin-bound paclitaxel) may be reduced to 100 mg/m² or 75 mg/m².

In some instances, Gal-9 antibody treatment may be initiated concomitantly with chemotherapy (e.g., gemcitabine and nab-paclitaxel). Alternatively, Gal-9 antibody treatment may be initiated after a chemotherapeutic regimen (e.g., gemcitabine and nab-paclitaxel) has already started. In some instances, Gal-9 antibody treatment is administered concomitantly with chemotherapy (e.g., gemcitabine and nab-paclitaxel), and subsequently chemotherapy is discontinued. In some instances, in which the chemotherapy is stopped, administration of anti-Gal-9 antibody treatment regimen may be continued.

In any of the above embodiments, the interval or cycle may be once every week. In any of the above embodiments, the interval or cycle may be once every 2 weeks. In some embodiments, the regimen may be once every 2 weeks for one cycle, once every 2 weeks for two cycles, once every 2 weeks for three cycles, once every 2 weeks for four cycles, or once every 2 weeks for more than four cycles. In some embodiments, the treatment may be once every 2 weeks for 1 to 3 months, once every 2 weeks for 3 to 6 months, once every 2 weeks for 6 to 12 months, or once every 2 weeks for 12 to 24 months, or longer.

In any of the above embodiments, the interval or cycle may be 3 weeks. In some embodiments, the regimen may be once every 3 weeks for one cycle, once every 3 weeks for two cycles, once every 3 weeks for three cycles, once every 3 weeks for four cycles, or once every 3 weeks for more than four cycles. In some embodiments, the treatment may be once every 3 weeks for 1 to 3 months, once every 3 weeks for 3 to 6 months, once every 3 weeks for 6 to 12 months, or once every 3 weeks for 12 to 24 months, or longer.

In any of the above embodiments, the interval or cycle may be 4 or more weeks. In some embodiments, the regimen is once every 4 or more weeks for one cycle, once every 4 or more weeks for two cycles, once every 4 or more weeks for three cycles, once every 4 or more weeks for four cycles, or once every 4 or more weeks for more than four cycles. In some embodiments, the treatment may be once every 4 or more weeks for 1 to 3 months, once every 4 or more weeks for 3 to 6 months, once every 4 or more weeks for 6 to 12 months, or once every 4 or more weeks for 12 to 24 months, or longer. In some embodiments, the treatment may be a combination of treatment at various time, e.g., a combination or 2 weeks, 3 weeks, 4 or more 4 weeks. In some embodiments, the treatment interval may be adjusted in accordance with the patient's response to treatment. In some embodiments, the dosage(s) is adjusted in accordance with the patient's response to treatment. In some embodiments, the dosages are altered between treatment intervals. In some embodiments, the treatment may be temporarily stopped. In some embodiments, anti-Galectin-9 therapy is temporarily stopped. In some embodiments, chemotherapy is temporarily stopped. In some embodiments, both are temporarily stopped. In any of these embodiments, the anti-Gal9 antibody may be G9.2-17 in IgG4 form as disclosed herein, having a heavy chain of SEQ ID NO: 19 and a light chain of SEQ ID NO: 15).

Response to treatment can also be characterized by one or more of immunophenotype in blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), PDL-1 expression (e.g., by immunohistochemistry), mismatch repair status, or tumor markers relevant for the disease (e.g., as measured at 3 months, 6 months or 12 months, or at a later time). Non-limiting examples of such tumor markers include Ca15-3, CA-125, CEA, CA19-9, alpha fetoprotein. These parameters can either be compared to baseline levels prior to initiation of treatment or can be compared to a control group as described herein.

In any of the methods disclosed herein, the subject may examined for one or more of the following features before, during, and/or after the treatment: (a) one or more tumor markers in blood samples from the subject, optionally wherein the one or more tumor markers comprise CA15-3, CA-125, CEA, CA19-9, and/or alpha fetoprotein, and any other tumor-type specific tumor markers; (b) cytokine profile; and (c) galectin 9 serum/plasma levels, d) peripheral blood mononuclear cell immunophenotyping, e) tumor tissue biopsy/excisional specimen multiplex immunophenotyping, f) tumor tissue biopsy/excisional specimen galectin-9 expression levels and pattern, g) any other immune score test such as: PDL-1 immunohistochemistry, tumor mutational burden (TMB), tumor microsatellite instability status, as well as panels such as: Immunoscore®—HalioDx, ImmunoSeq-Adaptive Biotechnologies, TIS, developed on the NanoString nCounter® gene expression system, 18-gene signature, PanCancer IO 360™ assay (NanoString Technologies) etc. Other suitable biomarkers specific to the target tumor such as PDAC may also be used.

In some embodiments, methods described herein, wherein a Gal-9 antibody is administered with a chemotherapy, e.g., gemcitabine and nab-paclitaxel, may modulate levels of immune cells and immune cell markers in the blood or in tumors. Such changes can be measured in patient blood and tissue samples using methods known in the art, such as multiplex flow cytometry and multiplex immunohistochemistry. For example, a panel of phenotypic and functional PBMC immune markers can be assessed at baseline prior to commencement of the treatment and at various time point during treatment. Table 2 lists non-limiting examples of markers useful for these assessment methods. Flow cytometry (FC) is a fast and highly informative method of choice technology to analyze cellular phenotype and function, and has gained prominence in immune phenotype monitoring. It allows for the characterization of many subsets of cells, including rare subsets, in a complex mixture such as blood, and represents a rapid method to obtain large amounts of data. Advantages of FC are high speed, sensitivity, and specificity. Standardized antibody panels and procedures can be used to analyze and classify immune cell subtypes. Multiplex IHC is a powerful investigative tool which provides objective quantitative data describing the tumor immune context in both immune subset number and location and allows for multiple markers to be assessed on a single tissue section. Computer algorithms an be used to quantify IHC-based biomarker content from whole slide images of patient biopsies, combining chromogenic IHC methods and stains with digital pathology approaches.

TABLE 2
PBMC phenotyping markers

| | PBMC phenotyping markers | | PBMC phenotyping markers |
|---|---|---|---|
| CD3 | Total T cells | CD16 | NK cells |
| CD4 | CD4+ T cells | CD11b | Monocytes/macrophages |
| CD8 | CD8+ T cells | CD11c | Monocytes/macrophages, DCs |
| CD25 | Tregactivation | CD14 | Monocyte subsets, macrophages |
| CD27 | T cell maturation; | CD33 | Total myeloid cells |
| | B cell naïve/memory | | |
| CD38 | T cell maturation; | FceR1a | Antigen presenting DC cells |
| | B cell naïve/memory | | |
| CD45RA | Naïve/memory cells | CD19 | Total B cells |
| CD45RO | Naïve/memory cells | T-bet | T cells subsets |
| CD56 | NKT/NK cells (T cell subset) | gdTCR | Gamme delta T cells |
| CD127 | T cell subsets | CD274 (PDL-1) | Checkpoint |
| CD152 (CLTA-4) | Checkpoint | Tim-3 | Checkpoint |
| CD279 (PD-1) | Checkpoint | TCRVa24-Ja18 | INKT calls |
| FoxP3 | Treg cells | Live/dead | General |
| HLA-DR | Activation/Antigen presentation | CD45 | General |

Accordingly, in some embodiments, the methods described herein, wherein an anti-gal9 antibody is administered in combination with a chemotherapy, may modulate immune activation markers such as those in Table 2. These markers can either be compared to baseline levels prior to initiation of treatment or can be compared to a control group receiving chemotherapy only, (e.g., at certain intervals, e.g., at 3 months, 6 months or 12 months). In some embodiments, cytokine profiles are modulated.

In some embodiments, the disclosure provides methods of modulating an immune response in a subject. The immune response may be T cell-mediated and/or B cell-mediated immune responses that are influenced by modulation of immune cell activity, for example, T cell activation. In one embodiment of the disclosure, an immune response is T cell mediated. As used herein, the term “modulating” means changing or altering, and embraces both upmodulating and downmodulating. For example “modulating an immune response” means changing or altering the status of one or more immune response parameter(s). Exemplary parameters of a T cell mediated immune response include levels of T cells (e.g., an increase or decrease in effector T cells) and levels of T cell activation (e.g., an increase or decrease in the production of certain cytokines). Exemplary parameters of a B cell mediated immune response include an increase in levels of B cells, B cell activation and B cell mediated antibody production.

When an immune response is modulated, some immune response parameters may decrease and others may increase. For example, in some instances, modulating the immune response causes an increase (or upregulation) in one or more immune response parameters and a decrease (or downregulation) in one or more other immune response parameters, and the result is an overall increase in the immune response, e.g., an overall increase in an inflammatory immune response. In another example, modulating the immune response causes an increase (or upregulation) in one or more immune response parameters and a decrease (or downregulation) in one or more other immune response parameters, and the result is an overall decrease in the immune response, e.g., an overall decrease in an inflammatory response.

In some embodiments, the methods described herein, wherein an anti-gal9 antibody is administered in combination with a chemotherapy, may modulate soluble galectin-9 levels in blood (serum or plasma), or galectin-9 tumor tissue expression levels and pattern of expression by immunohistochemistry (tumor, stroma, immune cells) in a subject, (e.g., as measured at 3 months, 6 months or 12 months, or at a later time). Galectin-9 levels in a subject can either be compared to baseline levels prior to initiation of treatment or can be compared to a control group, e.g., receiving chemotherapy alone.

In some embodiments, the methods described herein may decrease of one or more of soluble galectin-9 levels in blood (serum or plasma), or in galectin-9 tumor tissue expression levels and pattern of expression by immunohistochemistry (tumor, stroma, immune cells) decrease. (e.g., as measured at 3 months, 6 months or 12 months, or at a later time).

In some embodiments, methods described herein, wherein an anti-gal9 antibody is administered in combination with a chemotherapy, may modulate one or more tumor markers (increase or decrease) relevant for the disease (e.g., as measured at 3 months, 6 months or 12 months, or at a later time). Non-limiting examples of such tumor markers include Ca15-3, CA-125, CEA, CA19-9, alpha fetoprotein. These parameters can either be compared to baseline levels prior to initiation of treatment or can be compared to a control group, e.g., receiving chemotherapy alone.

In some embodiments, the methods provided herein, wherein an anti-gal-9 antibody is administered in combination with chemotherapy (e.g. gemcitabine and nab-paclitaxel), may improve the overall response (e.g., at 3, 6 or 12 months), e.g., as compared to a baseline level prior to initiation of treatment or as compared to a control group receiving chemotherapy along. In some embodiments, the methods provided herein may result in a complete response, a partial response or stable disease (e.g., as measured at 3 months, 6 months or 12 months according to RECIST or iRECIST criteria). In some embodiments, the methods may improve the likelihood of a complete response, a partial response or stable disease (e.g., as measured at 3 months, 6 months or 12 months), e.g., as compared to a control group receiving chemotherapy alone. In some embodiments, treating can result in longer survival or greater likelihood of survival, e.g., at a certain time, e.g., at 6 or 12 months or at a later time point.

In any of the methods described herein, Partial response, stable disease, complete response, a partial response, stable disease, progressive disease, disease progressing (e.g., as measured at 3 months, 6 months or 12 months, or at a later time), can be assessed according to RECIST criteria or iRECIST criteria.

In some embodiments, the methods provided herein, wherein an anti-gal-9 antibody is administered in combination with chemotherapy (e.g. gemcitabine and nab-paclitaxel), may increase the time to disease progression or increase the time in progression-free survival (e.g., as measured at 6 months) as compared to a control group, e.g., receiving chemotherapy alone. In some embodiments, treating can result in a greater likelihood of progression free survival (e.g., as measured at 3 months, 6 months or 12 months, or at a later time post initiation of treatment) as compared to a control group.

In some embodiments, the methods provided herein, wherein an anti-gal-9 antibody is administered in combination with chemotherapy (e.g. gemcitabine and nab-paclitaxel), may improve duration and depth of response according to RECIST 1.1 criteria, (e.g., as measured at 3 months, 6 months or 12 months, or at a later time post initiation of treatment) as compared to a control group, e.g., receiving chemotherapy alone.

In some embodiments, the methods provided herein, wherein an anti-gal-9 antibody is administered in combination with chemotherapy (e.g. gemcitabine and nab-paclitaxel), may improve quality of life and/or improving symptom control (e.g., as measured at 1 month, 3 months, 6 months or 12 months, or at a later time using ECOG scale) as compared to baseline prior to initiation of treatment or as compared to a control group.

A subject having a target solid tumor as disclosed herein, for example, PDAC, can be identified by routine medical examination, e.g., laboratory tests, organ functional tests, genetic tests, interventional procedure (biopsy, surgery) any and all relevant imaging modalities. In some embodiments, the subject to be treated by the method described herein is a human cancer patient who has undergone or is subjecting to an anti-cancer therapy, for example, chemotherapy, radiotherapy, immunotherapy, or surgery. In some embodiments, subjects have received prior immune-modulatory anti-tumor agents. Non-limiting examples of such immune-modulatory agents include, but are not limited to as anti-PD1, anti-PD-L1, anti-CTLA-4, anti-OX40, anti-CD137, etc. In some embodiments, the subject shows disease progression through the treatment. In other embodiments, the subject is resistant to the treatment (either de novo or acquired). In some embodiments, such a subject is demonstrated as having advanced malignancies (e.g., inoperable or metastatic). Alternatively, or in addition, in some embodiments, the subject has no standard therapeutic options available or ineligible for standard treatment options, which refer to therapies commonly used in clinical settings for treating the corresponding solid tumor.

In some instances, the subject may be a human patient having a refractory disease, for example, a refractory PDAC. As used herein, “refractory” refers to the tumor that does not respond to or becomes resistant to a treatment. In some instances, the subject may be a human patient having a relapsed disease, for example, a relapsed PDAC. As used herein, “relapsed” or “relapses” refers to the tumor that returns or progresses following a period of improvement (e.g., a partial or complete response) with treatment.

In some embodiments, the human patient to be treated by the methods disclosed herein may meet one or more of the inclusion and exclusion criteria disclosed in Example 2 below. For example the human patient may be older than 18 and have histologically confirmed unresectable metastatic cancer (e.g., adenocarcinomas and squamous cell carcinomas). The patient may have measurable disease, according to RECIST v. 1.1. In some instances, the human patient may have recent archival tumor sample (e.g., obtained within 5 years) available for biomarker analyses (e.g., galectin-9 tumor tissue expression, which may be assessed by IHC). In some instances, the human patient is an PDAC patient who has received at least one line of systemic therapy in the metastatic cancer setting. Such a patient may either be gemcitabine-containing regimen naïve or at least 6 months out of having been treated using a gemcitabine-containing regimen. The patient may have Eastern Cooperative Oncology Group (ECOG) performance status 0-1 and/or Karnofsky score>70. The patient may also have adequate hematologic and end organ function, e.g., neutrophil count≥1×10⁹/L, platelet count≥100×10⁹/L, for HCC in Part 1≥50×10⁹/L; hemoglobin≥8.5 g/dL without transfusion in the previous week, Creatinine≤1.5×ULN, AST (SGOT)≤3×ULN (≤5×ULN when HCC or hepatic metastases are present), ALT (SGPT)≤3×ULN (≤5×ULN when HCC or hepatic metastases present), Bilirubin≤1.5×ULN (patients with known Gilbert's disease may have a bilirubin≤3.0×ULN), Albumin≥3.0 g/dL, INR and PTT≤1.5×ULN; and/or amylase and lipase≤1.5×ULN. In some instances, the human patient shows no evidence of active infection or infections requiring parenteral antibiotics, and no serious infection within 4 weeks before the treatment starts. Pancreatic, biliary, or enteric fistulae allowed, provided they are controlled with an appropriate non-infected and patent drain.

Alternatively or in addition, the human patient subject to any treatment disclosed herein may be free of: (i) metastatic cancer of an unknown primary, (ii) clinically significant, active uncontrolled bleeding, any bleeding diathesis (e.g., active peptic ulcer disease); (iii) radiation therapy within 4 weeks of the first dose of the treatment, (iv) with fungating tumor masses or locally advanced PDAC; (v) ≥CTCAE grade 3 toxicity (except alopecia and vitiligo) due to prior cancer therapy; (v) history of second malignancy, (vi) evidence of severe or uncontrolled systemic diseases, congestive cardiac failure>New York Heart Association (NYHA) class 2, or myocardial infarction (MI) within 6 months, (vii) serious non-healing wound, active ulcer, or untreated bone fracture; (viii) uncontrolled pleural effusion, pericardial effusion, or ascites requiring recurrent drainage procedures; (ix) history of severe allergic, anaphylactic, or other hypersensitivity reactions to chimeric or humanized antibodies or fusion proteins; (x) significant vascular disease (e.g., aortic aneurysm requiring surgical repair or recent arterial thrombosis) within 6 months of the treatment, history of pulmonary embolism, stroke or transient ischemic attack within 3 months prior to the treatment, and/or history of abdominal fistula or gastrointestinal perforation within 6 months prior to the treatment; (xi) active auto-immune disorder (except type I diabetes, hypothyroidism requiring only hormone replacement, vitiligo, psoriasis, or alopecia); (xii) requires systemic immunosuppressive treatment; (xii) tumor-related pain (>grade 3) unresponsive to broad analgesic interventions (oral and/or patches); (xiii) uncontrolled hypercalcemia, despite use of bisphosphonates; (xiv) received organ transplant(s).

In some instances, the subject is a human patient having an elevated level of Galectin-9 as relative to a control level. The level of Galectin-9 can be a plasma or serum level of Galectin-9 in the human patient. In other examples, the level of Galectin-9 can be the level of cell-surface Galectin-9, for example the level of Galectin-9 on cancer cells. In one example, the level of Galectin-9 can be the level of surface Galectin-9 expressed on cancer cells in patient-derived organotypic tumor spheroids (PDOT), which can be prepared by, e.g., the method disclosed in Examples below. A control level may refer to the level of Galectin-9 in a matched sample of a subject of the same species (e.g., human) who are free of the solid tumor. In some examples, the control level represents the level of Galectin-9 in healthy subjects.

To identify such a subject, a suitable biological sample can be obtained from a subject who is suspected of having the solid tumor and the biological sample can be analyzed to determine the level of Galectin-9 contained therein (e.g., free, cell-surface expressed, or total) using conventional methods, e.g., ELISA or FACS. In some embodiments, organoid cultures are prepared, e.g., as described herein, and used to assess Galectin-9 levels in a subject. Single cells derived from certain fractions obtained as part of the organoid preparation process are also suitable for assessment of Galectin-9 levels in a subject. In some instances, an assay for measuring the level of Galectin-9, either in free form or expressed on cell surface, involves the use of an antibody that specifically binds the Galectin-9 (e.g., specifically binds human Galectin-9). Any of the anti-Galectin-9 antibodies known in the art can be tested for suitability in any of the assays described above and then used in such assays in a routine manner. In some embodiments, an antibody described herein (e.g., an G9.2-17 antibody) can be used in such as assay. In some embodiments, an antibody described co-pending U.S. patent application Ser. No. 16/173,970 and in co-owned, co-pending International Patent Application PCT/US18/58028, the relevant disclosures of each of which are incorporated by reference for the purpose and subject matter referenced herein. In some examples, the anti-Galectin-9 antibody is a Fab molecule. Assay methods for determining Galectin-9 levels as disclosed herein are also within the scope of the present disclosure.

### Kits for Use in Combined Therapy of Solid Tumors

The present disclosure also provides kits for use in treating or alleviating a solid tumor, for example, PDA, CRC, HCC, or cholangiocarcinoma, and others described herein. Such kits can include one or more containers comprising an anti-Galectin-9 antibody, e.g., any of those described herein (e.g., G9.2-17(IgG4)), and optionally one or more chemotherapeutics (e.g., a gemcitabine and/or paclitaxel) to be co-used with the anti-Galectin-9 antibody, which is also described herein.

In some embodiments, the kit can comprise instructions for use in accordance with any of the methods described herein. The included instructions can comprise a description of administration of the anti-Galectin-9 antibody, and the one or more chemotherapeutics, to treat, delay the onset, or alleviate a target disease as those described herein. In some embodiments, the kit further comprises a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-Galectin-9 antibody and the one or more chemotherapeutics generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the solid tumor. In some embodiments, instructions are provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container may be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is an anti-Galectin-9 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention are employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. In Vivo Study of Anti-Galectin-9 Antibody in Combination with Chemotherapeutics for Cancer Treatment in a Pancreatic Cancer Mouse Model

Preclinical assessment of an anti-galectin-9 IgG4 fully human antibody (G9.2-17(IgG4)) for the treatment of difficult to treat solid tumors—as a single agent or in combination with other systemic chemotherapeutic anti-cancer modalities—was performed in a mouse model of pancreatic cancer.

The specific animal used was the orthotopic mPA6115 pancreatic cancer xenograft model in female C57BL/6 mice. To generate this model, first, tumors were sourced from mPA6115 mice, a mouse homograft model of pancreatic ductal adenocarcinoma (PDAC) that retains morphological similarity to human PDAC. The mPA6115 mouse stain carried the conditional mutant Kras (Kras^{LSL-G12D/WT}), a constitutive deletion of Trp53 (P53KO/KO) and a Cre driven by the promotor of Pdxl gene, and developed severe PDAC tumors at the age of 8 weeks.

At that time, mPA6115 mice with palpable tumors were sacrificed, and their pancreatic tumors were collected. The collected tumor tissue was cut into small fragments (˜2 mm³) and transplanted subcutaneously (SC) to the syngeneic recipients, C57BL/6 mice. These seed tumors were maintained subcutaneously in the C57BL/6 mice until the volume of seed tumor reached 700-1000 mm³. Once seed tumors reached the desired volume, the tumors were collected and cut into pieces of about 2 mm³ in diameter. Tumors then were washed with ice cold Roswell Park Memorial Institute (RPMI) 1640 medium (without serum) to remove the adjacent non-tumor tissues. Then the tumor pieces were placed in ice cold RPMI 1640 medium until orthotopic implantation. The same day that seed tumors were collected, 6-7 week old female C57BL/6 mice were subjected to pancreatic orthotopic implantation. Specifically, after animals were fully anesthetized, a small longitudinal incision below the left lower rib cage was made to expose the spleen and the pancreas underneath the spleen. One seed tumor piece per mouse was sewn into the pancreas with 6-0 silk suture. Then the tissue surrounding the tumor piece was sutured with 6-0 silk suture, and the tumor piece was wrapped with pancreas tissue. The abdomen was then closed with a 4-0 silk suture. After tumor implantation, animals were kept in a warm cage, and subsequently returned to the animal room after full recovery from the anesthesia.

On the day when implantation was performed, implemented mice were randomly grouped into 6 groups based on their body weight where randomization was performed based on the “Matched distribution” method (StudyDirector™ software, version 3.1.399.19). The date of randomization was denoted as day 0. Three days after implantation, animals began a dosing regimen according to group number. The dosing regimen for each group is provided below in Table 3.

TABLE 3
Study Dosing Schedule

| | | Dose | Dosing | Dosing | | Dosing |
|---|---|---|---|---|---|---|
| Group | | level | Solution | Volume | Route of | Frequency |
| No. | Treatment | (mg/kg) | (mg/ml) | (μL/g) | Admin. | & Duration |
| 1 | Untreated | — | — | — | — | — |
| 2 | chemo vehicle | — | — | 10 | i.p. | QW*5 |
| | control, saline | | | | | |
| 3 | Isotype IgG1 mouse | 200 | 2 | 100 | i.p. | Q4D*8 |
| | | ug/mouse | | μL/mouse | | |
| 4 | Anti-Gal9 mAb | 200 | 2 | 100 | i.p. | Q4D*14 |
| | | ug/mouse | | μL/mouse | | |
| 5 | Gemcitabine | 50 | 5 | 10 | i.p. | QW*6 |
| | Abraxane | 15 | 1.5 | 10 | i.v. | QW*6 |
| 6 | Anti-Gal9 mAb | 200 | 2 | 100 | i.p. | Q4D*16 |
| | | ug/mouse | | μL/mouse | | |
| | Gemcitabine | 50 | 5 | 10 | i.p. | QW*10 |
| | Abraxane | 15 | 1.5 | 10 | i.v. | QW*10 |

| | |
|---|---|
| 7 | 10 non-tumor bearing mice for blood sampling |

i.p. = intraperitoneal; i.v. = intravenous; QW = once a week; Q4D = once every four days

For these studies, an anti-galectin-9 mouse IgG1 was used. This antibody, referred to as Anti-Gal9 mAb, was the mouse IgG1 version of the human G9.2-17 antibody, which binds the same carbohydrate binding domain 2 (CRD2) on galectin-9 as G9.2-17 and has the same VH and VL regions as G9.2-17. Thus, resulting data using Anti-Gal9 mAb is correlative to human efficacy of G9.2-17. In addition to treating mice with only Anti-Gal9 mAb (group 4), groups 5 and 6 of implanted mice were also treated with a standard of care chemotherapy (a gemcitabine/abraxane regimen), or a combination of Anti-Gal9 mAb and chemotherapy.

After pancreatic orthotopic implantation, the mice in groups 1-7 were checked daily for morbidity and mortality. During routine monitoring, the animals were checked for any effects of tumor growth and treatments on behavior such as mobility, food and water consumption, body weight gain/loss, eye/hair matting and any other abnormalities. Body weights and tumor volumes measured twice per week after randomization using StudyDirector™ software (version 3.1.399.19). Measurements and monitoring were collected as described from day 0 until day 66 when the last mouse was found dead. Blood, plasma, spleen, and tumors were collected from each mouse at end of life. Table 4 below shows the average life span of the mice by experiment group. The longest survival in all of the control arms (Groups 1, 2 and 3) was day 33, while the last mouse died on day 55, 41, and 66, in Group 4 (anti-galectin-9 IgG1), 5 (gemcitabine/abraxane), 6 (combination therapy), respectively.

TABLE 4
Average life span for mice per group

| | | | day, when the | mean |
|---|---|---|---|---|
| Group | | median, | last mouse | survival |
| No. | Treatment | days | died/euthanized | day |
| Group 1 | Untreated | 27 | 33 | 27 |
| Group 2 | chemo vehicle | 26 | 33 | 28 |
| | control, saline | | | |
| Group 3 | Isotype IgG1 | 31 | 33 | 28 |
| | mouse | | | |
| Group 4 | Anti-Gal9 | 27 | 55 | 32 |
| | mAb | | | |
| Group 5 | Gemcitabine/ | 31 | 41 | 28 |
| | Abraxane | | | |
| Group 6 | Anti-Gal9 | 31 | 66 | 34 |
| | mAb/ | | | |
| | Gemcitabine/ | | | |
| | Abraxane | | | |

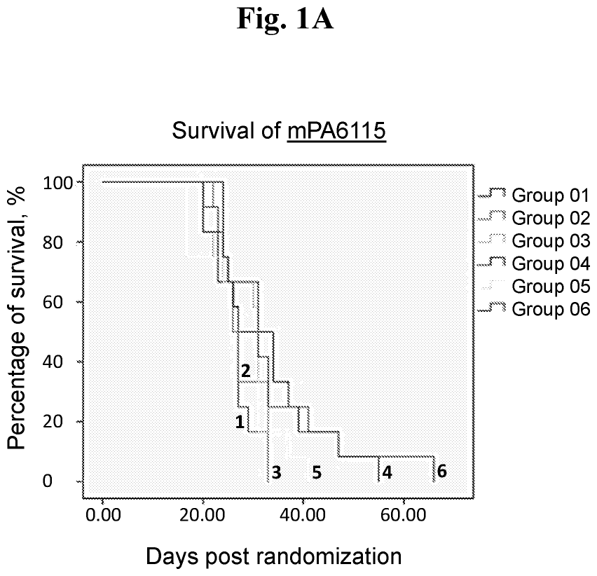

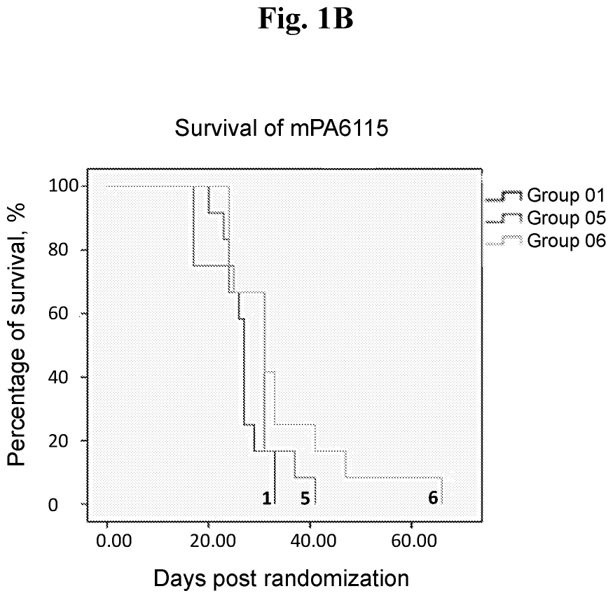

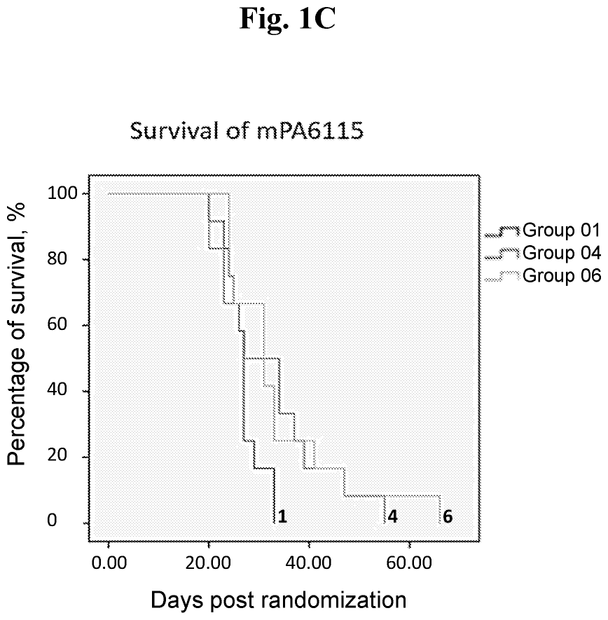

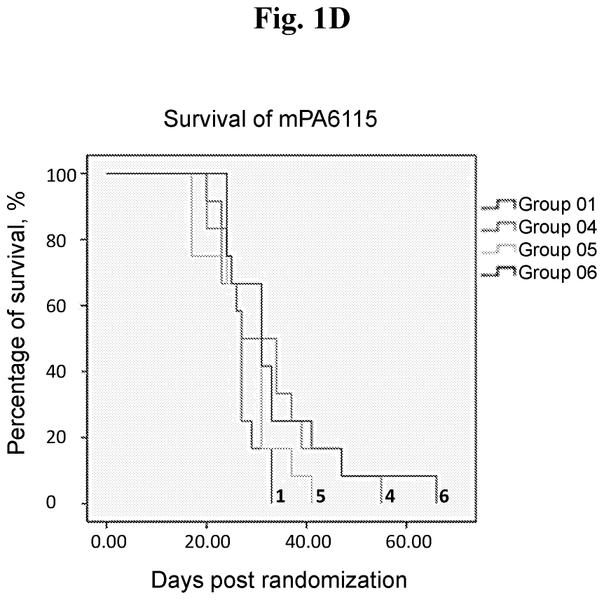

The primary endpoint of survival in animals engrafted with orthotopic KPC tumors was assessed by estimating survival curves for each group, considered separately, using the Kaplan-Meier method and compared statistically using the log rank test. Specifically, Kaplan-Meier survival curves/Log Rank test (SPSS 18) were used. The Kaplan-Meier survival curves and log rank test are shown in FIGS. 1A-1D. Results of log rank test are provided in Table 5.

TABLE 5
Log rank test

| Log rank test | Group No. | P value |
|---|---|---|
| Group 1 VS | 2 | 0.663 |
| | 3 | 0.233 |
| | 4 | 0.069 |
| | 5 | 0.134 |
| | 6 | 0.051 |
| Group 2 VS | 3 | 0.826 |
| | 4 | 0.091 |
| | 5 | 0.515 |
| | 6 | 0.156 |
| Group 3 VS | 4 | 0.094 |
| | 5 | 0.791 |
| | 6 | 0.193 |
| Group 4 VS | 5 | 0.349 |
| | 6 | 0.733 |
| Group 5 VS | 6 | 0.220 |

*, p < 0.05;
**, p < 0.01;
***, p < 0.001;
ns, p ≥ 0.05

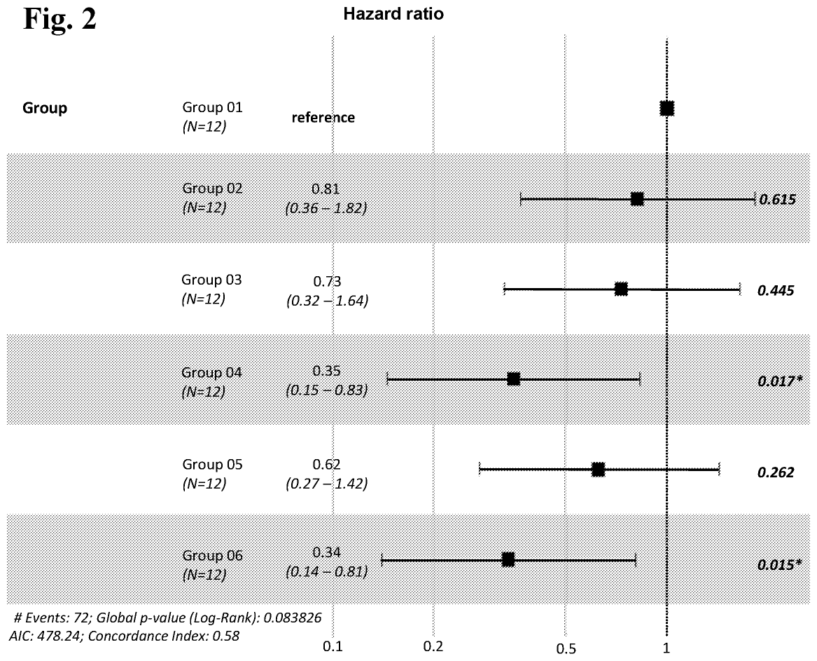

Cox-regression analysis (coxph function of survival R package) was used to calculate hazard ratios (HR) and their 95% confidence interval (%95CI) of group 4-6 against group 1, group 2 and group 3 respectively. We also used cox-regression analysis to calculate hazard ratios (HR) and their 95% confidence interval (%95CI) of group 5 and 6 against group 4. Finally, we used cox-regression analysis to calculate hazard ratio (HR) and its 95% confidence interval (%95CI) of group 6 against group 5. Results of the cox regression analysis are shown in FIG. 2 and Table 6.

TABLE 6
Cox regression analysis

| | Compared | | | |
|---|---|---|---|---|
| Group | against | p. value | HR | HR (95% CI) |
| Group 4 | Group 1 | 0.017 | 0.348 | (0.146, 0.83) |
| Group 5 | Group 1 | 0.262 | 0.624 | (0.274, 1.422) |
| Group 6 | Group 1 | 0.015 | 0.336 | (0.14, 0.806) |
| Group 4 | Group 2 | 0.052 | 0.427 | (0.181,1.009) |
| Group 5 | Group 2 | 0.525 | 0.767 | (0.339, 1.738) |
| Group 6 | Group 2 | 0.045 | 0.413 | (0.173, 0.982) |
| Group 4 | Group 3 | 0.091 | 0.477 | (0.202,1.125) |
| Group 5 | Group 3 | 0.707 | 0.856 | (0.379, 1.932) |
| Group 6 | Group 3 | 0.079 | 0.460 | (0.193, 1.094) |
| Group 5 | Group 4 | 0.166 | 1.795 | (0.784 , 4.109) |
| Group 6 | Group 4 | 0.933 | 0.966 | (0.424 , 2.198) |
| Group 6 | Group 5 | 0.149 | 0.538 | (0.232, 1.248) |
| Group 2 | Group 1 | 0.615 | 0.813 | (0.364, 1.818) |
| Group 3 | Group 1 | 0.445 | 0.730 | (0.325, 1.639) |
| Group 3 | Group 2 | 0.790 | 0.897 | (0.402, 2.002) |

*, p < 0.05;
**, p < 0.01;
***, p < 0.001;
ns, p ≥ 0.05

For the cox regression analysis that used group 1 as the reference, group 4 and group 6 had significant lower hazard ratio than group 1, whereas group 2 and group 3 did not have significant different hazard ratios with group 1. In the cox regression analysis that used group 2 as the reference, group 6 had a significant lower hazard ratio than group 2; however, group 3 did not have significant different hazard ratios with group 2. For the cox regression analysis that used group 3 as the reference, groups 4, 5, and 6 did not have significant different hazard ratios with group 3. In the cox regression analysis that used group 4 as the reference, groups 5 and 6 did not have significant different hazard ratios with group 4. Finally, the cox regression analysis that used group 5 as the reference showed that group 6 did not have significant different hazard ratios with group 5.

These data demonstrated that the combination of the anti-galectin-9 antibody and gemcitabine/abraxane is well tolerated, can be administered over prolonged periods of time (max administered 16 doses for the anti-galectin-9 IgG1 antibody (mouse IgG1 version) and 10 doses for gemcitabine/abraxane), and delivered survival benefit over untreated animals (Group 6 vs Group 1: Cox analysis, HR=0.336, HR(95% CI)=(0.14, 0.806), p=0.015; as well as p=0.051 Log Rank test for mean survival). Anti-galectin-9 IgG1 alone delivered survival benefit over untreated animals (Group 4 vs Group 1: Cox analysis, HR=0.348, HR(95% CI)=(0.146, 0.83), p=0.017).

No tumor was found in the pancreas from the last mouse in Group 6 on day 66 when the mouse was found dead, terminating the study. Based on the historical data, the take rate of orthotopic mPA6115 model in vehicle groups were 100%. As such, the last mouse in Group 6 was a complete responder to the combination anti-galectin-9/gemcitabine/abraxane regimen.

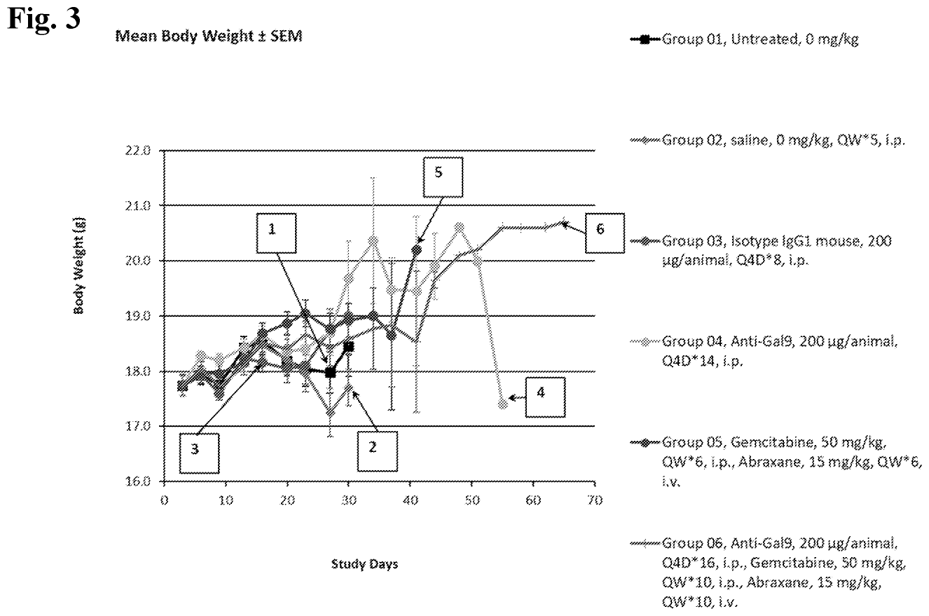

Body weights were measured in mice engrafted with orthotopic KPC tumors twice per week after implantation/randomization (day 0) until all mice were euthanized or died. FIG. 3 shows the body weight measurements collected for the duration of the study period measured by using StudyDirector™ software (version 3.1.399.19). The last mouse in Group 4 was moribund and euthanized on day 55 with the tumor weight of 2544.6 mg (TV=1877.07 mm³). From day 51 to 55, there was only one last mouse in Group 4 with body weight change from 12.36% to −2.25% compared with the weight at the first day of treatment. The body weight loss was most likely be correlated to the condition induced by tumor growth.

Overall, the data in this example confirmed the safety and efficacy of the anti-galectin-9 regimen and the combination anti-galectin-9/gemcitabine/abraxane regimen in the orthotopic Pancreatic Cancer Xenograft Model mPA6115.

### Example 2: A Phase 1a/1b Open Label, Multi-center Study of the Safety, Pharmacokinetics, and Anti-tumor Activity of G9.2-17(IgG4) Alone and in Combination with Chemotherapy in Subjects with Metastatic Solid Tumors

Galectin-9 is a molecule overexpressed by many solid tumors, including those in pancreatic cancer, colorectal cancer, and hepatocellular carcinoma. Moreover, Galectin-9 is expressed on tumor-associated macrophages, as well as intra-tumoral immunosuppressive gamma delta T cells, thereby acting as a potent mediator of cancer-associated immunosuppression. As described herein, monoclonal antibodies targeting Galectin-9 (e.g., G9.2-17) have been developed. Data have demonstrated that G9.2-17 halts pancreatic tumor growth by 50% in orthotopic KPC models and extended the survival of KPC animals by more than double. In addition, the anti-Galectin-9 antibody shows signals of therapeutic synergy with chemotherapeutics in animal studies.

The purpose of this Phase I/II multicenter study is to determine the safety, tolerability, maximum tolerated dose (MTD), and objective tumor response after 12 to 24 months of treatment in subjects having metastatic solid tumors, e.g., pancreatic adenocarcinoma (PDA), colorectal cancer (CRC), hepatocellular carcinoma (HCC), or cholangiocarcinoma (CCA). The study also examines progression-free survival (PFS), the duration of response (by RESIST), disease stabilization, the proportion of subjects alive, as well as pharmacokinetic (PK) and pharmacodynamics (PD) parameters. Subjects undergo pre- and post-treatment biopsies, as well as PET-CT imaging pre-study and once every 8 weeks for the duration of the study. In addition, immunological endpoints, such as peripheral and intra-tumoral T cell ratios, T cell activation, macrophage phenotyping, cytokine profiling in serum, tumor immunohistochemistry, and Galectin-9 serum levels are examined. The study is performed under a master study protocol, and the study lasts for 12-24 months.

Subject, disease, and all clinical and safety data are presented descriptively as means, medians, or proportions, with appropriate measures of variance (e.g., 95% confidence interval range). Waterfall and Swimmers plots are be used to graphically present the ORR and duration of responses for subjects for each study arm, within each disease site, as described below. Exploratory correlations analysis are also be undertaken to identify potential biomarkers that may be associated with ORR. All statistical analyses are performed using SAS, version 9.2 (SAS, Cary, NC).

### (A) Study Design

The lowest anticipated pharmacologically active dose (PAD) is currently estimated to be 2 mg/kg, based on the mouse model KPC004 data, in which 50 mcg/mouse (2 mg/kg; human equivalent dose HED=0.16 mg/kg) was established as an active dose. Alternate models were active at the 200 or 400 mcg/mouse dose range (8-16 mg/kg; HED=0.65-1.3 mg/kg).

Table 7 below shows proposed clinical starting dose levels dependent upon the outcome of the repeat-dose GLP-compliant toxicity study at the proposed dose levels of 100 and 200 mg/kg G9.2-17. The estimated starting doses use either 1/10 of the no observed adverse effect level (NOAEL) or ⅙ of the highest non-severely toxic dose (HNSTD) as a starting point and then convert that dose in mg/kg to the HED in mg/kg.

TABLE 7
Proposed Clinical Starting Doses

| Dose Level | | | | 3-Fold Safety | |
|---|---|---|---|---|---|
| in GLP | | HED of the | 1/10 of | factor added to | 1/6 of |
| Toxicity | | GLP toxicity | NOAEL | 1/10 NOAEL | HNSTD |
| Study | | study dose | (HED) | (HED) | (HED) |
| mg/kg | Species | mg/kg | mg/kg | mg/kg | mg/kg |
| 300 | Rat | 48 | 30 (5) | 10 (1.6) | 50 (8) |
| 300 | NHP | 97 | 30 (10) | 10 (3.2) | 50 (16) |

Proposed clinical starting dose with additional 3-fold safety factor
based on the outcome of the GLP repeat-dose toxicity study in NHP

| | | | | |
|---|---|---|---|---|
| 300 | NHP | 6 | 2 | 3.6 |

This study includes both monotherapy of G9.2-17 (IgG4) and combination therapy including G9.2-17 and gemcitabine/Abraxane ((paclitaxel protein-bound particles for injectable suspension; albumin-bound). The study is split into 2 parts: Part 1 (Phase 1a) and Part 2 (Phase 1b).

Part 1

Part 1 of the study is a dose-finding study using a continuous reassessment method (CRM) (O'Quigley et al., 1990), a model-based design that informs how the dosage of G9.2-17 should be adapted for the next patient cohort based on past trial data. Two patients at a time are dosed with G9.2-17 alone, with a maximum available sample size of 24. Patients receive 5 dose levels every 2 weeks until progression of disease, unacceptable toxicity, or withdrawal from the study development of dose-limiting toxicity (DLT). The dose levels are:
Dose level 1=2 mg/kg;
Dose level 2=4 mg/kg;
Dose level 3=8 mg/kg;
Dose level 4=12 mg/kg; and
Dose level 5=16 mg/kg.

The dosing regimen is once every two weeks (Q2W) by intravenous (IV) administration. Dose reduction of up to 25% may be adopted when needed.

As a safety precaution, at each dose escalation, new patients are entered and treated only after the first patient of each cohort has been treated with G9.2-17 and after a minimum 7 days post-treatment have elapsed. Part 1 is complete after six consecutive patients have received the same dose and that dose is identified as the optimal biological dose (OBD).

Part 2

Part 2 of the study is a Simon's two-stage optimal design (six arms: pancreatic ductal adenocarcinoma (PDA), CRC, and Cholangio carcinoma). The study investigates the use of the G9.2-17 alone (single agent arms of the study) and in conjunction with gemcitabine/Abraxane. The dose of the anti-Galectin-9 antibody used is below the level found to exhibit toxicity in Part 1.

The optimal two-stage design is used to test the null hypothesis that the ORR≤5% versus the alternative that the ORR≥15% within the single agent arms. After testing the drug on 23 patients in the first stage, the respective trial arm is terminated if ≤1 patients respond. If the trial goes on to the second part of Simon's optimal design, a total of 56 patients are enrolled into each of the single agent arms. If the total number responding patients is ≤5, the investigational drug within that arm is rejected. If ≥6 patients have an ORR at 3 months, the expansion cohort for that arm is activated. The above approach is applied to the single agent arms of the study.

Combination Treatment with G9.2-17 and Gemcitabine Abraxane

Combination treatment with G9.2-17 and gemcitabine/Abraxane is evaluated in patients with metastatic PDAC. The primary objective of this study is progression free survival (PFS) at 6 months. Secondary objectives include improvements in objective response rate (ORR), disease control rate (DCR) at 6 and 12 months, patient survival at 6 and 12 months, time to response, duration and depth of response by RECIST 1.1 criteria, safety and tolerability. In the case of the combination arms, the starting dose of G9.2-17 is administered at one dose lower than the OBD identified in Part 1 (e.g., the RP2D dose level identified in Part 1). Doses of gemcitabine/Abraxane follow those on FDA-approved label and may be adjusted in light of regimen specific side effects, if any (e.g., 2 weeks on one week off). If 3 or more patients develop a DLT, the dose of G9.2-17 is reduced in a stepwise manner not to exceed dose 3 amounts unless low doses continue to provide a clinical benefit.

In the patient cohort consisting of metastatic PDAC patients, the primary efficacy endpoint is PFS at 6 months. In the 1st line metastatic setting using gemcitabine/Abraxane, the 6 months PFS was reported to be 50% (Von Hoff et al., 2013). After testing the G9.2-17/chemotherapy combination on the first 11 patients in the first stage, the trial is terminated if 6 or fewer patients exhibit PFS≥6 months. In the second stage of the trial, a total of 25 patients are studied. If the total number of responding patients with PFS of ≥6 months is ≤16, the study arm is rejected.

Expansion of cohorts is implemented where an early efficacy signal has been detected. Once a promising efficacy signal is identified within one of the five trial arms that is attributable to the tumor type, an expansion cohort is launched to confirm the finding. The sample size for each of the expansion arms is determined based on the point estimates determined in Part 2, in combination with a predetermined level of precision for the 95% confidence interval (95% CI) around the ORR/patient survival.

Part 3

Part 3 includes expansion of cohorts where early efficacy signal has been detected. If a promising efficacy signal is identified within one of the trial arms that is attributable to the tumor type, an expansion cohort is launched to confirm the finding. The sample size for each of the expansion arms is determined based on the point estimates determined in Part 2, in combination with predetermined level of precision for the 95% confidence interval (95% CI) around the ORR.

The study duration is 12-24 months.

### (B) Patient Population

Patients with relapsed/refractory metastatic cancers, irrespective of tumor type, are eligible for the dose-finding study using the continual reassessment method (CRM) as described by O'Quigley (1990). Expansion is envisaged in PDAC where mode of action and/or an early efficacy signal are captured in Part 1.

Patient inclusion and exclusion criteria are the same for both Part 1 and part 2.

### Patient Inclusion Criteria:

1. Written informed consent (mentally competent patient, able to understand and willing to sign the informed consent form)
2. Age≥18 years, male or non-pregnant female
3. Histologically confirmed unresectable metastatic cancer (adenocarcinomas and squamous cell carcinomas allowed). Patients with resectable disease are excluded.
4. Able to comply with the study protocol
5. Life expectancy>3 months
6. Recent archival tumor sample (obtained within 5 years) available for biomarker analyses.
7. Patient able and willing to undergo pre- and on/post-treatment biopsies. The planned biopsies should not expose the patient to substantially increased risk of complications. Every effort is made that the same lesion is biopsied on repeat biopsies.
8. Measurable disease, according to RECIST v1.1. Note that lesions biopsied should not be target lesions.
9. Expected survival>3 months
10. For Part 1: No available standard of care options, or patient has declined available and indicated standard of care therapy, or is not eligible for available and indicated standard of care therapy. For Part 2:
   PDAC expansion cohort—first line metastatic patients who are either gemcitabine-containing regimen naïve or at least 3 months out of having been treated using a gemcitabine-containing regimen previously in a neoadjuvant or adjuvant/locally advanced setting.
   CCR and CCA expansion cohorts—received at least one prior line of therapy in the metastatic setting.
11. Coronavirus SARS-CoV-2 (COVID-19) negative patients. Vaccination for COVID-19 is allowed before or during the study period. Information on timing and type of vaccine must be recorded.
12. Eastern Cooperative Oncology Group (ECOG) performance status 0-1 and/or Karnofsky score>70.
13. High microsatellite instability (MSI-H) and microsatellite stability (MSS) patients are allowed for Part 1 of the study.
14. Adequate hematologic and end organ function, defined by the following laboratory results obtained prior to first dose of study drug treatment, provided no anti-cancer treatment was administered within the last 7 days: neutrophil count≥1×10⁹/L, platelet count≥100×10⁹/L, for HCC in Part 1≥50×10⁹/L; hemoglobin≥9.0 g/dL without transfusion in the previous week, Creatinine≤1.5×ULN, AST (SGOT)≤3×ULN (≤5×ULN when HCC or hepatic metastases are present), ALT (SGPT)≤3×ULN (≤5×ULN when HCC or hepatic metastases present), Bilirubin≤1.5×ULN (patients with known Gilbert's disease may have a bilirubin≤3.0×ULN), Albumin≥3.0 g/dL, INR and PTT≤1.5×ULN; amylase and lipase≤1.5×ULN
15. No evidence of active infection or infections requiring parenteral antibiotics, and no serious infection within 4 weeks before study start.
16. Women of child-bearing potential must have a negative pregnancy test prior to study entry.
17. For women of childbearing potential: agreement to remain abstinent (refrain from heterosexual intercourse) or to use contraceptive methods that result in a failure rate of <1% per year during the treatment period and for at least 180 days after the last study treatment.

A woman is of childbearing potential if she is post-menarche, has not reached a postmenopausal state (≥12 continuous months of amenorrhea with no identified cause other than menopause), and has not undergone surgical sterilization (removal of ovaries and/or uterus).

Examples of contraceptive methods with a failure rate of <1% per year include bilateral tubal ligation, male sterilization, hormonal contraceptives that inhibit ovulation, hormone-releasing intrauterine devices and copper intrauterine devices. The reliability of sexual abstinence should be evaluated in relation to the duration of the clinical trial and the preferred and usual lifestyle of the patient. Periodic abstinence (eg, calendar, ovulation, symptom-thermal, or post ovulation methods) and withdrawal are not acceptable methods of contraception. Fertile men must practice effective contraceptive methods during the study, unless documentation of infertility exists.
18. Four (4) weeks or 5 half-lives (whichever is shorter) since the last dose of anti-cancer therapy before the first G9.2-17 administration
19. Continuation of bisphosphonate treatment (zoledronic acid) or denosumab for bone metastases which have been stable for at least 6 months before C1D1 is allowed.
20. For Part 1: Hepatocellular carcinoma that progressed while receiving at least one previous line of systemic therapy, including sorafenib, lenvatinib, nivolumab, atezolizumab and bevacizumab, or who are intolerant to or refused sorafenib treatment following progression on standard therapy including surgical and/or local regional therapies, or standard therapy considered ineffective, intolerable, or inappropriate or for which no effective standard therapy is available.
21. Biliary or gastric outlet obstruction allowed, provided it is effectively drained by endoscopic, operative, or interventional means.
22. Pancreatic, biliary, or enteric fistulae allowed, provided they are controlled with an appropriate non-infected and patent drain (if any drains or stents are in situ, patency needs to be confirmed before study start).

Patient Exclusion Criteria:
1. Patient diagnosed with metastatic cancer of an unknown primary.
2. Patient unwilling or unable to follow protocol requirements
3. Prior or current illicit drug addiction (medical and recreational marijuana/CBD/THC is not considered “illicit”)
4. Clinically significant, active uncontrolled bleeding, and any patients with a bleeding diathesis (e.g., active peptic ulcer disease). Prophylactic or therapeutic use of anticoagulants is allowed.
5. Pregnant and/or lactating females
6. Receiving any other investigational agents or participating in any other clinical trial involving another investigational agent for treatment of solid tumors within 4 weeks or 5 half-lives of the administered drug (whichever is shorter) prior to Cycle 1, Day 1 of the study, or other investigational therapy or major surgery within 4 weeks of the date of consent, or planned surgery within 4 weeks of envisaged study start (this includes dental surgery).
7. Radiation therapy within 4 weeks of the first dose of study drug, except for palliative radiotherapy to a limited field, such as for the treatment of bone pain or a focally painful tumor mass, and which does not jeopardize required measurable lesions for response assessment (RECIST v1.1).
8. Patients with fungating tumor masses
9. Patients with locally advanced PDAC without distant organ metastatic deposits
10. ≥CTCAE grade 3 toxicity (except alopecia and vitiligo) due to prior cancer therapy. Grade 4 immune-mediated toxicities with a prior checkpoint inhibitor. Grade 2 or Grade 3 pneumonitis or any other Grade 3 checkpoint inhibitor-related toxicity that led to immunotherapy treatment discontinuation. Low-grade (<Grade 3) toxicities, such as neuropathy from prior treatments, manageable electrolyte abnormalities, lymphopenia, alopecia, and vitiligo are allowed.
11. History of second malignancy, except those treated with curative intent more than five years previously without relapse or low likelihood of recurrence (for example, non-melanotic skin cancer, cervical carcinoma in situ, early (or localized) prostate cancer, or superficial bladder cancer)
12. Evidence of severe or uncontrolled systemic diseases, congestive cardiac failure>New York Heart Association (NYHA) class 2, myocardial infarction (MI) within 6 months, or laboratory finding that in the view of the Investigator makes it undesirable for the patient to participate in the trial
13. Any medical condition that the Investigator considers significant to compromise the safety of the patient or that impairs the interpretation of G9.2-17 toxicity assessment
14. Serious non-healing wound, active ulcer, or untreated bone fracture
15. Uncontrolled pleural effusion, pericardial effusion, or ascites requiring recurrent drainage procedures. For the purposes of this study, “recurrent” is defined as ≥3 drains in the last 30 days.
16. History of severe allergic, anaphylactic, or other hypersensitivity reactions to chimeric or humanized antibodies or fusion proteins
17. Significant vascular disease (e.g., aortic aneurysm requiring surgical repair or recent arterial thrombosis) within 6 months of Cycle 1, Day 1
18. History of pulmonary embolism, stroke or transient ischemic attack within 3 months prior to Cycle 1, Day 1
19. History of abdominal fistula or gastrointestinal perforation within 6 months prior to Cycle 1, Day 1
20. Active auto-immune disorder (except type I/II diabetes, hypothyroidism requiring only hormone replacement, vitiligo, psoriasis, or alopecia areata)
21. Requires systemic immunosuppressive treatment including, but not limited to cyclophosphamide, azathioprine, methotrexate, thalidomide, and anti-tumor necrosis factor [anti-TNF] agents. Patients who have received or are receiving acute, low dose systemic immunosuppressant medications (e.g., ≤10 mg/day of prednisone or equivalent) may be enrolled. Replacement therapy (e.g., thyroxine, insulin, physiologic corticosteroid replacement therapy [e.g., ≤10 mg/day of prednisone equivalent] for adrenal or pituitary insufficiency) is not considered a form of systemic treatment. The use of inhaled corticosteroids and mineralocorticoids (e.g., fludrocortisone), topical steroids, intranasal steroids, intra-articular, and ophthalmic steroids is allowed
23. Severe tumor-related pain (Grade 3, Common Terminology Criteria for Adverse Events [CTCAE] v.5.0) unresponsive to broad analgesic interventions (oral and/or patches)
24. Hypercalcemia (defined as ≥Grade 3, per CTCAE v 5.0) despite use of bisphosphonates
25. Any other diseases, metabolic dysfunction, physical examination finding, or clinical laboratory finding giving reasonable suspicion of a disease or condition that contraindicates the use of an investigational drug or that may affect the interpretation of the results or render the patient at high risk of treatment complications
26. Received organ transplant(s)
27. Patients undergoing dialysis
28. For part 1, hormonal androgen deprivation therapy allowed to continue for subjects with metastatic castrate resistant pancreatic cancer.
29. Active brain or leptomeningeal metastases. Patients with brain metastases are eligible provided they have shown clinically and radiographically stable disease for at least 4 weeks after definitive therapy and have not used steroids (>10 mg/day of prednisone or equivalent) for at least 4 weeks prior to the first dose of study drug
30. For patients enrolled into nivolumab combination cohorts, no prior exposure to any anti PD-1 or anti-PD-L1 agent in any prior lines of therapy. Additionally, patients diagnosed as dMMR/MSI-H are excluded.

### Additional Exclusion Criteria for Subjects Having (Hepato) Biliary Cancers (HCC):

1. Any ablative therapy (Radio Frequency Ablation or Percutaneous Ethanol Injection) for HCC<6 weeks prior trial entry
2. Hepatic encephalopathy or severe liver adenoma
3. Child-Pugh score≥7
4. Metastatic hepatocellular carcinoma that progressed while receiving at least one previous line of systemic therapy, including sorafenib, or who are intolerant to or refused sorafenib treatment following progression on standard therapy, including surgical and/or local regional therapies, or standard therapy considered ineffective, intolerable, or inappropriate or for which no effective standard therapy is available
5. Biliary or gastric outlet obstruction allowed provided it is effectively drained by endoscopic, operative, or interventional means
6. Pancreatic, biliary, or enteric fistulae allowed provided they are controlled with an appropriate non-infected and patent drain (if any drains or stents are in situ, patency needs to be confirmed before the study start).

A patient shall discontinue the treatment if one or more of the following occur:
Pregnancy
Unmanageable toxicity
Symptomatic deterioration attributed to disease progression as determined by the investigator after integrated assessment of radiographic data, biopsy results, and clinical status.
Intolerable toxicity related to G9.2-17, including development of an irAE determined by the investigator to be unacceptable given the individual patient's potential response to therapy and severity of the event
Any medical condition that may jeopardize the patient's safety if he or she continues on study treatment
Use of another non-protocol anti-cancer therapy

### (C) Objectives

### Part 1 (Phase 1a)

Primary Objective(s): Safety, tolerability, optimal biological dose (OBD) or maximum administered dose (MAD), recommended Phase 2 dose (RP2D)
Secondary Objective(s): Pharmacokinetic (PK), pharmacodynamic (PD) parameters, immunogenicity
Exploratory Objective(s): Exploratory end points for Part 1, in addition to exploratory end points listed below: Objective Response Rate (ORR), disease control rate (DCR), progression free survival (PFS), patient survival at 3 months (for Part 1), 6 and 12 months (for Parts 1 and 2).

### Part 2 in CRC and CCA (Phase 1b)

Primary Objective(s): Objective Response Rate (ORR)
Secondary Objective(s): Progression free survival (PFS), disease control rate (DCR), duration and depth of response by RECIST 1.1, patient survival at 6 and 12 months, time to response, safety and tolerability

### Part 2 in PDAC (Phase 1b)

Primary Objective(s): Progression free survival (PFS) at 6 months
Secondary Objective(s): Objective Response Rate (ORR), disease control rate (DCR) at 6 and 12 months, patient survival at 6 and 12 months, time to response, duration and depth of response by RECIST 1.1 criteria, safety and tolerability

### Exploratory Endpoints for all Study Parts:

iRECIST criteria, immunophenotyping from blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), PDL-1 expression by immunohistochemistry, mismatch repair status, tumor markers relevant for the disease, ctDNA, and correlation of these parameters with response. Time to response (TTR). Quality of life and symptom control.

### (D) Study Procedures

(i) Schedule of Assessments

The schedule of assessments is divided into 4-week cycles after the pre-dose 1 cycle 1 screening, which may take place up to 4 weeks prior to commencement of treatment. Table 8 lists the pre-dose screening assessments and tests, as well as indicating those to be conducted during the treatment cycles. Optional visits are allowed during each cycle, if medically indicated, during which any of the study assessments may be performed.

(ii) Screening and Assessment Procedures

The following procedures (outlined in Table 8. Schedule of Assessments) must be conducted within 4 weeks of initiating treatment:
Written informed consent
Verify inclusion and exclusion criteria
Record of prior COVID-19 infection and latest RT PCR and/or SARS CoV2 IgG/IgM test results, if performed
Record patient's intention to receive a COVID-19 vaccine if and when available
Record vaccination status for seasonal flu
Tumor imaging assessment CT with or without contrast is preferred, MRI with or without contrast if required based on investigator's judgement, PET-CT (diagnostic CT) if required based on investigator's judgement)
Tumor biopsy (pre dose 1 and repeat biopsy)—scheduled depending upon scan(s)
For the pre-screen assessment of galectin-9 expression by immunohistochemistry, archival tumor tissue may be used, if available, provided it was acquired within a 5-year time frame and details of treatment (s) administered post tissue acquisition are known and documented. This is not a pre-requisite for enrollment and investigators will endeavor to provide archival specimens whenever possible.
Relevant tumor marker per tumor type—e.g., CA15-3, CA-125, CEA, CA19-9, alpha fetoprotein, etc., will be assessed every cycle pre-dose (which may be decreased to every 3 cycles after 6 months of treatment, following the same schedule as restaging scans), as appropriate.
Patient demographics
Personal medical history, including prior treatments/surgeries, record of any implants in situ or past implants, prior and/or current use of medical devices, concomitant medications (name, indication, dose, route, start and end dates dose modifications if any and reason), pre-existing symptoms, and adverse events), hereditary diseases at risk of based on family history and complete family history to the best knowledge of the patient
Any and all additional test results previously acquired (next generation and/or whole exome sequencing results, circulating tumor DNA testing, germline sequencing results, DPD test results, G6PD test results, Oncotype Dx and/or Endopredict test results, consensus molecular subtypes (CMS) classification, DXA scans, if available. These are not a pre-requisite for enrollment.
Record of any dental surgery/root canal or ophthalmology surgery performed in the past 12 months
History of mandibular or maxillary necrosis
History of any prior port-a-cath infections requiring intravenous antibiotics and/or anti-fungals, port-a-cath replacements.
If the patient is deemed dehydrated according to investigator's assessment, oral and/or i.v. rehydration is allowed and advised prior to dosing at any cycle. Investigator may decide to order a BUN test on the day of dosing to guide decision making.
Physical examination and visual sign recording
ECHO, Ejection fraction (EF)
12-lead ECG
Record of the site and status/dimensions of any keloid scars
In patients with stable and pre-treated brain metastases, perform a neurological exam
Record of any dietary requirements or preferences (for example, practice of a particular diet regimen: intermittent fasting, keto diet etc.)
Use of supplements (current and with the past 12 months), type duration of use, dose and frequency
ECOG and/or Karnofsky status
Vital signs (temperature, HR, BP, RR, including weight) post-supine for 5 minutes
Pregnancy test, if female of childbearing age. If prior history of bilateral salpingo-oophorectomy and/or hysterectomy then not needed but record these surgical procedures.
Allergies past and/or present (allergen, severity)
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4, lipase, amylase, PTH, FSH, LH, CRP, and/or troponin:
Blood coagulation (PT, PTT, APTT)
Urinalysis
PD blood—biomarker analysis
PD tumor—biomarker analysis on the pre-treatment biopsy

(iii) On-Study Procedures

Ensure that PD blood biomarker analyses are done at each blood draw; and tumor biomarker analyses are done on pre- and on/post-study biopsies. Screening process must involve and document a neurological exam. Any >grade 2 irAEs will be referred to the relevant specialist and documented accordingly. Management of irAEs will be conducted according to: Management of Immunotherapy-Related Toxicities, NCCN Guidelines Version 1.2020. Study-related procedures and assessments performed during on-study treatment are detailed as follows and in Table 8, Schedule of Assessments.

For COVID-19 infection diagnosed while on treatment, investigators and the Sponsor will follow FDA guidelines and local policies, and the investigator should contact the medical monitor to discuss best course of action.

Cycle 1 Procedures

(a) Cycle 1, Day 1 Procedures

The following procedures will be performed on Day 1 after all the previous screening and baseline procedures have been completed.
12-lead ECG
Physical examination
ECOG
Vital signs (temperature, HR, BP, RR, including weight and/or BSA) post-supine for 5 minutes
Concomitant medications (name, indication, dose, route, start and end dates, any and all dose modifications, timing thereof and reason)
Adverse events
Complete blood count (CBC), differential, platelets, hemoglobin
All within 7 days of C1D1: Blood chemistry (glucose, Hgb Alc (if history of DM1 or DM2), total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)—Fasting glucose will be taken pre-dose, only if clinically indicated. TSH, fT4, lipase, amylase, PTH, FSH, LH, CRP, troponin
Relevant tumor marker—e.g., CA15-3, CA-125, CEA, CA19-9, alpha fetoprotein, etc., will be assessed every cycle pre-dose (which may be decreased to every 3 cycles after 6 months of treatment, following the same schedule as restaging scans), as appropriate
Blood coagulation (PT, PTT, APTT)
Urinalysis (including protein and culture/antibiogram)
PD blood—biomarker analysis (soluble galectin-9, tissue IHC for galectin-9 from pre-treatment biopsy and immunophenotyping)
PK blood samples at time points as annotated in Table 8.

(b) Cycle 1, Day 2 Procedures

The following procedures will be performed on Day 2 of Cycle 1.
Concomitant medications (name, indication, dose, route, start and end dates)
Adverse events
PD blood—biomarker analysis
PK blood samples

(c) Cycle 1, Day 4 Procedures

The following procedures will be performed on Day 4 of Cycle 1.
Concomitant medications (name, indication, dose, route, start and end dates)
Adverse events
PD blood—biomarker analysis
PK blood samples

(d) Cycle 1, Day 7 Procedures

The following procedures are performed on Day 7 of Cycle 1.
Vital signs (temperature, HR, BP, RR, including weight) post-supine for 5 minutes
Concomitant medications
Adverse events
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)
PD tumor—biomarker analysis
PK blood samples

(e) Cycle 1, Day 15 Procedures

The following procedures are performed on Day 15 of Cycle 1.
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK) ECOG
Vital signs (temperature, HR, BP, RR, including weight) post-supine for 5 minutes
Adverse events
PD blood—biomarker analysis
PK blood samples

Cycle 2 Procedures

(a) Cycle 2, Day 1 Procedures

The following procedures are performed on Day 1 of Cycle 2.
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4
Relevant tumor marker—e.g., CA15-3, CA-125, CEA, CA19-9, alpha fetoprotein, etc., will be assessed every cycle pre-dose (which may be decreased to every 3 cycles after 6 months of treatment, following the same schedule as restaging scans), as appropriate.
Physical examination
Adverse events
ECOG
Vital signs (temperature, HR, BP, RR, including weight) post-supine for 5 minutes
PK blood samples
PD blood—biomarker analysis
Concomitant medications (name, indication, dose, route, start and end dates, any and all dose modifications, timing thereof and reason)

(b) Cycle 2, Day 7 Procedures

The following procedures will be performed on Day 7 of Cycle 2.
12-lead ECG
Vital signs (temperature, HR, BP, RR, including weight) post-supine for 5 minutes
Concomitant medications (name, indication, dose, route, start and end dates)
Adverse events
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)
PD blood—biomarker analysis
PK blood samples
Pregnancy test, if female of childbearing age and ovaries and uterus in situ

(c) Cycle 2, Day 15 Procedures

The following procedures are performed on Day 15 of Cycle 2.
Restaging scan (CT with contrast, MRI, PET-CT or X-ray)—may be done 6-8 weeks from onset of study drug administration, scheduled as an additional separate visit
Tumor biopsy −3/+12 days if feasible and scheduled as a separate visit/can coincide with the scan as imaging guidance may be required to facilitate obtaining the tissue sample (target lesion should not be biopsied)
PD tumor—biomarker analyses
ECOG
Vital signs (temperature, HR, BP, RR, including weight) post-supine for 5 minutes
Adverse events
PK blood samples
PD blood—biomarker analysis

Cycle 3 Procedures

(a) Cycle 3, Day 1 Procedures

The following procedures are performed on Day 1 of Cycle 3.
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4
Relevant tumor marker—e.g., CA15-3, CA-125, CEA, CA19-9, alpha fetoprotein, etc., will be assessed every cycle pre-dose (which may be decreased to every 3 cycles after 6 months of treatment, following the same schedule as restaging scans), as appropriate.
Physical examination
ECOG
Vital signs (temperature, HR, BP, RR, include weight) post-supine for 5 minutes
Concomitant medications (name, indication, dose, route, start and end dates)
Adverse events
PD blood—biomarker analysis
PK blood samples
Pregnancy test, if female of childbearing age

(b) Cycle 3, Day 7 Procedures

The following procedures are performed on Day 7 of Cycle 3.
ECHO
Vital signs (temperature, HR, BP, RR, including weight) post-supine for 5 minutes
Concomitant medications (name, indication, dose, route, start and end dates)
Adverse events
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)
PD blood—biomarker analysis
PK blood samples

(c) Cycle 3, Day 15 Procedures

The following procedures are performed on Day 15 of Cycle 3.
ECOG
Vital signs (temperature, HR, BP, RR, including weight) post-supine for 5 minutes
Adverse events
PK blood samples
PD blood—biomarker analysis

Cycle 4, and Beyond, Procedures

(a) Cycle 4, Day 1 Procedures

The following procedures are performed on Day 1 of Cycle 4 and subsequent cycles.
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4, lipase, amylase, PTH, troponin, FSH, LH, CRP
Restaging scan (CT with contrast, MRI, PET-CT or X-ray)—may be done 6-8 weeks from onset of study drug administration
Relevant tumor marker—e.g., CA15-3, CA-125, CEA, CA19-9, alpha fetoprotein, etc., will be assessed every cycle pre-dose (which may be decreased to every 3 cycles after 6 months of treatment, following the same schedule as restaging scans), as appropriate
Physical examination
ECOG
Adverse events
PK blood samples
PD blood—biomarker analysis
Pregnancy test, if female of childbearing age

(b) Cycle 4, Day 7 Procedures (same as C3 D7)

The following procedures will be performed on Day 7 of Cycle 4

(c) Cycle 4, Day 15 Procedures (same as C3D15)

The following procedures will be performed on Day 15 of Cycle 4

(iv) End of Study or Early Termination Procedures

The following procedures are done on Day 59 or thirty days after the last dose, including patients who have discontinued treatment early.
Restaging scan (CT with or without contrast id preferred, MRI with or without contrast, PET-CT if required by investigator)—repeat if end of study is >6 to 8 weeks after last cycle and in shorter intervals, at investigator's discretion
Relevant tumor marker—e.g., Ca15-3, CA-125, CEA, CA19-9, alpha fetoprotein, etc., will be assessed every cycle pre-dose (which may be decreased to every 3 cycles after 6 months of treatment, following the same schedule as restaging scans), as appropriate
12-lead ECG
Physical examination
ECOG
Vital signs (temperature, HR, BP, RR, including weight) post-supine for 5 minutes
Concomitant medications (name, indication, dose, route, start and end dates)
Adverse events
Pregnancy test, if female and ovaries and uterus in situ
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4, PTH, Estradiol. prolactin, testosterone, FSH, LH
Blood coagulation (PT, PTT)
Urinalysis
PD blood—biomarker analysis
PK blood samples

(v) Long-Term Follow-Up

Once a patient has completed the treatment period, overall survival follow-up will be performed every 3 months for up to 2 years. Radiological assessment will continue, where possible, for patients withdrawing due to clinical progression.

Survival data as well as information on any new anticancer therapy initiated after disease progression will be collected approximately every 3 months. Follow-up may be performed by telephone interview or chart review and will be reported on the case report form. During the Follow-Up Period, deaths, regardless of causality, and serious adverse events thought to be related to study treatment will be collected and reported within 24 hours of discovery or notification of the event.

(vi) Study Assessments

(a) Physical Examination

Medical and physical examinations must be performed by a qualified physician, nurse practitioner, or physician assistant, and should include a thorough review of all body systems at Screening, during treatment, and at End of Study. Physical examinations include a breast examination, if clinically indicated, as well as vital signs—temperature, heart rate (HR), blood pressure (BP), respiratory rate (RR)—measured after resting in a supine position for 5 minutes. Patient weight will also be measured and recorded.

(b) Medical History

The medical history includes oncology history, radiation therapy history, surgical history, current and past medication.
Personal medical history, including prior treatments/surgeries, record of any implants in situ or past implants, prior and/or current use of medical devices, concomitant medications (name, indication, dose, route, start and end dates dose modifications if any and reason), pre-existing symptoms, and adverse events), hereditary diseases at risk of based on family history and complete family history to the best knowledge of the patient
Any and all additional test results previously acquired (next generation and/or whole exome sequencing results, circulating tumor free DNA testing, germline sequencing results, DPD test results, G6PD test results, Oncotype Dx and/or Endopredict test results,)
Record of any dental work performed in the past 12 months
Record of the site and status/dimensions of any keloid scars
For patients with previously resected pancreatic adenocarcinoma, record whether the primary tumor was localized to the head of pancreas, pancreatic body or the pancreatic tail.
Bowel habits/typical frequency and consistency

(c) Clinical Laboratory Evaluations

Patients have blood samples collected for routine clinical laboratory testing, according to the Schedule of Assessments. The clinical laboratory parameters will be analyzed at the site's local laboratory. Laboratory assessments to be completed will include hematology and serum chemistry and will be defined as following:
Serum Chemistry: To include glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, HgbAlc, blood urea nitrogen, CPK, TSH, fT4, lipase, amylase, PTH, testosterone, estradiol. prolactin, FSH, LH, and CRP. Fasting glucose is taken pre-dose on C1D1, C2D1, C3D1, C4D1 and on additional days, only if clinically indicated.
Hematology: To include complete blood count, differential, platelets, and hemoglobin
Coagulation: To include prothrombin time (PT) and partial thromboplastin time (PTT), activated partial thromboplastin time (APTT)
Biomarker Analysis (PD Blood): To include galectin-9 levels in patient serum/plasma, peripheral blood immunophenotyping, cytokine measurement.
Pharmacokinetic (PK) Blood Sampling: If the Investigator determines that the dose of study drug should be interrupted, additional PK, and safety assessments will be collected pre-dose (within 2 hours of dosing) & 4 hours+/−30 minutes post study drug. Centers that are not able to hold patients more than 2 hours post dose due to COVID-19 restrictions, will contribute samples at 2 hrs post dose only.

If administration is interrupted for any reason and then resumed, additional PK assessments may be performed during the interruption at the discretion of the Investigator. If the dose of study drug is reduced upon resuming administration, additional PK assessments will be collected pre-resumption of administration and at 2 hours+/−15 minutes post dosing completion. Additional PK and other blood assessments may be taken if clinically indicated at the discretion of the Investigator.

Blood for additional PK or PD assessments may be obtained approximately every 7 to 14 days, when possible, for up to 4 weeks after last study drug administration in patients who discontinue the study. Blood for PK assessment will be collected pre-dose, at 2 hours+/−15 minutes post completion of dosing) and 4, (+/−15 minutes) post-study drug administration.

(d) Urinalysis

Patients will have urine samples collected for routine urinalysis. The urinalysis will include color, appearance, and dipstick for specific gravity, protein, white blood cell-esterase, glucose, ketones, urobilinogen, nitrite, WBC, RBC, and pH, and urine culture at screening.

(e) Electrocardiogram (ECG)

The following parameters from 12-lead electrocardiograms will be evaluated: heart rate, PR interval, QRS duration, QT interval, and QTcF interval.

(f) Tumor Imaging Assessment

CT with contrast is the preferred modality (MRI, PET-CT and/or other imaging modalities instead of or in addition to the CT scan if CT is not feasible or appropriate, given location of the disease). Assessment should include the neck/chest/abdomen/pelvis at a minimum and should include other anatomic regions as indicated, based on the patient's tumor type and disease history. Imaging scans must be de-identified and archived in their native DICOM format as part of the patient study file. While the type of scan obtained is at the discretion of the Investigator as appropriate for the disease, the same method should be used for the duration of the study. Assessments are done every 6 to 8 weeks+/−1 week and at the End of Treatment if not assessed within the last 4 to 6 weeks.

(g) Tumor Biopsies

Pre and on/post-treatment biopsies are collected. Pre-treatment to be collected before administration of Dose 1. On treatment may be collected on any treatment day after Cycle 1 where a biopsy is feasible. Preferred next biopsy would be before the first on-study scan. In instances where the procedure cannot be performed within the protocol-specified timeframe, alternatives may be permitted but must be discussed with the Study Director/Medical Monitor. It is recognized that a variety of clinical factors may make it difficult to obtain adequate specimens. Decisions not to complete biopsy on-treatment should be discussed with the Medical Monitor.

(h) Tumor Markers

Exploratory markers, e.g., CA15-3, CA-125, CEA, CA19-9, alpha fetoprotein, etc., will be assessed every cycle pre-dose (which may be decreased to every 3 cycles after 6 months of treatment, following the same schedule as restaging scans), as appropriate.

(i) Adverse Events

Adverse events (AEs) starting or worsening after study drug administration will be recorded. AEs should be followed until resolved to baseline, stabilized, or deemed irreversible. All serious AEs (SAEs) must be collected from the date of patient's written consent until 30 days post-discontinuation of dosing or patient's participation in the study, if the last scheduled visit occurs a later time.

TABLE 8
Schedule of Assessments

| | | | | | | | | | | | | | | | | | | | | Post-Dose |
|---|---|---|---|---|---|---|---|---|---|---|---|---|---|---|---|---|---|---|---|---|
| | | Treatment Phase | | | | | | | | | | | | | | | | | | End of Study |
| | Pre-Dose | | | | | | | | | | | | | | | Cycle 4 (28 Days) and | | | | or Early |
| | Screening | Cycle 1 (28 Days) | | | | | | Cycle 2 (28 Days) | | | | Cycle 3 (28 Days) | | | | beyond (every 14 Days) | | | | Termination |
| | Study Day | | | | | | | | | | | | | | | | | | | |
| | | | | | | | Optional | | | | Optional | | | | Optional | | | | Optional | 30 days |
| | −28 to −1 | 1 | 2** | 4** | 7** | 15 | Visit^{@} | 1 | 7** | 15 | Visit^{@} | 1 | 7** | 15 | Visit^{@} | 1 | 7** | 15 | Visit^{@} | after last dose |
| | Name of Cycle Day | | | | | | | | | | | | | | | | | | | |
| | | C1D1 | C1D2 | C1D4 | C1D7 | C1D15 | C1DXX | C2D1 | C2D7 | C2D15 | C2DXX | C3D1 | C3D7 | C3D15 | C3DXX | CXD1 | CXD7 | CXD15 | CX4DXX | |
| | +/− days allowable | | | | | | | | | | | | | | | | | | | |
| | | 1 | 1 | 1 | 1 | | na | 2 | 2 | | na | 2 | 2 | | na | 2 | 2 | | na | 3 |
| Study Drug | | X | | | | X | | X | | X | | X | | X | | X | | X | | |
| administered | | | | | | | | | | | | | | | | | | | | |
| Disease Assessment | | | | | | | | | | | | | | | | | | | | |
| Restaging Scan (CT | X | | | | | | | | X | | | | | | | X | | | | X |
| with or without | | | | | | | | | +/−3 | | | | | | | +/−3 | | | | repeat if |
| contrast is preferred, | | | | | | | | | days | | | | | | | days | | | | end of |
| MRI with or without | | | | | | | | | | | | | | | | | | | | study is >6 |
| contrast, PET-CT | | | | | | | | | | | | | | | | | | | | to 8 weeks |
| if requested by the | | | | | | | | | | | | | | | | | | | | after last |
| investigator)^{B} may | | | | | | | | | | | | | | | | | | | | cycle |
| be done 6-8 weeks | | | | | | | | | | | | | | | | | | | | |
| from onset of study | | | | | | | | | | | | | | | | | | | | |
| drug administration | | | | | | | | | | | | | | | | | | | | |
| Tumor Biopsy^{C} | X^{&} | | | | | | | | | | | | | | | X^{&} | | | | |
| schedule will | | | | | | | | | | | | | | | | | | | | |
| depend on the scan | | | | | | | | | | | | | | | | | | | | |
| Relevant Tumor | X | | | | | X | | X _{D} | | X | | X^{D} | | X | | X^{D} | | X | | X |
| Markers^{D} | | | | | | | | | | | | | | | | | | | | |
| Study Procedures & | | | | | | | | | | | | | | | | | | | | |
| Examinations | | | | | | | | | | | | | | | | | | | | |
| Eligibility | X | | | | | | | | | | | | | | | | | | | |
| Assessment & | | | | | | | | | | | | | | | | | | | | |
| Informed Consent | | | | | | | | | | | | | | | | | | | | |
| Demographics^{E} | X | | | | | | | | | | | | | | | | | | | |
| Medical History^{F} | X | | | | | | | | | | | | | | | | | | | |
| ECHO^{G} | X | | | | | | | | | | | | | | | X | | | | |
| 12-lead ECG (QTcF) | X | X | | | | | | X | x | | | X | | | | X | | | | X |
| Physical Exam^{H} | X | X | | | | | | X | | X | | X | | X | | X | | X | | X |
| ECOG | X | X | | | | X | | X | | X | | X | | X | | X | | X | | X |
| Vital Signs (Temp, | X | X | | | x | X | | X | x | X | | X | | X | | X | | X | | X |
| HR, BP, RR- | | | | | | | | | | | | | | | | | | | | |
| include weight) | | | | | | | | | | | | | | | | | | | | |
| post-supine x 5 | | | | | | | | | | | | | | | | | | | | |
| minutes | | | | | | | | | | | | | | | | | | | | |
| Concomitant | X | X | X | X | X | X | | X | X | X | X | X | X | X | | X | X | X | | X |
| Medications^{I} | | | | | | | | | | | | | | | | | | | | |
| Adverse Events^{J} | | X | X | X | X | X | | X | X | X | X | X | X | X | | X | X | X | | X |
| Labs | | | | | | | | | | | | | | | | | | | | |
| Pregnancy Test^{K} | X | | | | | | | | | | | | | | | | | | | X |
| Blood Hematology^{L} | X | X | | | x | X | | X | x | X | | X | | X | | X | | X | | X |
| Chemistry^{M} | X | X | | | x | X | | X | x | X | | X | | X | | X | | X | | X |
| Coagulation, Glucose | X | X | | | | X | | X | | X | | X | | X | | X | | X | | X |
| & Urinalysis^{N} | | | | | | | | | | | | | | | | | | | | |
| Research Studies | | | | | | | | | | | | | | | | | | | | |
| (PD/PK) | | | | | | | | | | | | | | | | | | | | |
| PD Blood-Biomarker | X | X | x | x | x | X | | X | x | X | | X | | X | | X | | X | | X^{J} |
| Analysis^{O} | | | | | | | | | | | | | | | | | | | | |
| Exploratory | X | X | | | | X | | X | | X | | X | | X | | X | | X | | X |
| Biomarkers (Soluble | | | | | | | | | | | | | | | | | | | | |
| Galectin-9 and | | | | | | | | | | | | | | | | | | | | |
| Immunophenotyping) | | | | | | | | | | | | | | | | | | | | |
| PK Blood Samples ^{P} | X | X | X | X | X | X | | X | X | X | | X | X | X | | X | X | X | | X^{J} |

^{A}Study Drug Administration: treatment will be administered, and assessments performed as an outpatient for 4 hours on Day 1 of Cycle 1 and days xxx.
^{B}Restaging Scans (CT, MRI, PET-CT or x-ray): CT with contrast is the preferred modality (MRI if CT is not feasible or appropriate given location of the disease). Assessment should include the neck/chest/abdomen/pelvis at a minimum and should include other anatomic regions as indicated based on the patient's tumor type and/or disease history. Imaging scans must be de-identified and archived in their native DICOM format as part of the patient study file. While the type of scan obtained is at the discretion of the Investigator as appropriate for the disease, the same method should be used for the duration of the study. Assessments are done every 6 to 8 weeks +/−1 week and at the End of Treatment if not assessed within the last 4 to 6 weeks.
^{C}Tumor Biopsies: pre and on/post-treatment biopsies are collected. Pre-treatment to be collected before administration of Dose 1. On-treatment may be collected on any treatment day after Cycle 1 where a biopsy is feasible. Preferred next biopsy would be before first on-study scan. In instances where the procedure cannot be performed within the protocol-specified timeframe, alternatives may be permitted but must be discussed with the Study Director/Medical Monitor. It is recognized that a variety of clinical factors may make it difficult to obtain adequate specimens. Decisions not to complete biopsy on-treatment should be discussed with the Medical Monitor.
^{D}Relevant Tumor Markers: Exploratory Markers, e.g., Ca15-3, CA-125, CEA, CA19-9, alpha fetoprotein etc. will be assessed every cycle pre-dose (which may be decreased to every 3 cycles after 6 months of treatment, following the same schedule as restaging scans), as appropriate.
^{E}Demographics: includes date of birth, sex, height, race, ethnicity.
^{F}Medical History: includes oncology history, radiation therapy history, surgical history, current and past medication
^{G}MUGA/ECHO: repeat test will be collected, only if clinically indicated while on study.
^{H}Physical Exam: includes breast exam if clinically indicated
^{I}Concomitant Medications: name, indication, dose, route, start and end dates will be collected.
^{J}Adverse Events: starting or worsening after study drug administration will be recorded. AEs should be followed until resolved to baseline, stabilized or deemed irreversible. All SAEs must be collected from the date of patient's written consent until 30 days post discontinuation of dosing or patient's participation in the study, if the last scheduled visit occurs at a later time.
^{K}Pregnancy Test: Must have HCG sensitivity ≤IU/L or equivalent units of HCG and within 24 hours of first treatment cycle)
^{L}Blood Hematology: complete blood count, differential, platelets, hemoglobin
^{M}Biochemistry: glucose, total protein, albumin, electrolytes [sodium, potassium, chloride, total CO₂], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK
^{N}Coagulation, Glucose and Urinalysis: PT, PTT, Glucose and UA are collected. Collections at *Cycle 3 and beyond will be done only if clinically indicated (e.g. signs of bleeding, especially GI bleeding). ** Fasting Glucose will be taken pre-dose on C1D1, C3D1 and on additional days, only if clinically indicated
^{O}PD Blood-biomarker analysis: Gene expression, metabolites, oxygen consumption rate (OCR), other biomarker analysis and PDX development. Additional cycles to be performed on the same schedule as restaging scans.
^{P} PK Blood samples: If the Investigator determines that the dose of study drug should be interrupted, additional PK, and safety assessments will be collected pre-dose (within 2 hours of dosing) and 4 hours +/−30 minutes post study drug administration upon resumption of dosing; additional PK assessments may be performed during the interruption at the discretion of the Investigator. If the dose of study drug is reduced, additional PK assessments will be collected pre-dose (within 2 hours of dosing) & after starting the reduced study drug dose. Additional PK, and other blood assessments may be taken if clinically indicated at the discretion of the Investigator. PK Blood samples Blood for additional PK and/or PD assessments may be obtained ~every 7 to 14 days, when possible, for up to 4 weeks after last study drug administration in patients who discontinue the study. In addition to the time points indicated in the Schedule of Assessments, blood for additional PK assessments may be obtained at the discretion of the Investigator.
^{@}Optional visits are allowed during each cycle, if medically indicated, during which time any of the study assessments may be performed.
§ G9.2-17 will be administered every two weeks
Dosing: Dose will be administered to patient on Cycle 1 Day 1 and Cycle 1 Day 15; and will follow this schedule thereafter.
¹ collected pre-dose (within 2 hours of dosing) and 4 hours (+/−30 minutes) post-study drug administration
2 collected pre-dose (within 2 hours of dosing) and 2, 4, 6, 8 and 12 hours (+/−15 mins) post-study drug administration
³ collected pre-dose (within 2 hours of dosing)

### (E) Safety Assessments

All observed or volunteered adverse events regardless of treatment group or causal relationship to study drug will be recorded on the adverse event page(s) of the case report form (CRF). Adverse events will be coded using the MedDRA coding system and all AEs will be graded according to the National Cancer Institute Common Terminology Criteria for Adverse Events, version 5.0 (NCI-CTCAE) [NCI, 2017].

Adverse Events

An adverse event is defined in the International Conference on Harmonisation (ICH) Guideline for Good Clinical Practice as “any untoward medical occurrence in a patient or clinical investigation subject administered a pharmaceutical product and that does not necessarily have a causal relationship with this treatment.”

This definition of adverse events is broadened in this study to include any such occurrence (e.g., sign, symptom, or diagnosis) or worsening of a pre-existing medical condition from the time that a subject has signed informed consent to the time of initiation of the investigational drug. Worsening indicates that the pre-existing medical condition (e.g., diabetes, migraine headaches, gout, hypertension, etc.) has increased in severity, frequency, or duration of the condition or an association with significantly worse outcomes.

For all adverse events, the investigator must pursue and obtain information adequate to both determine the outcome of the adverse event and to assess whether it meets the criteria for classification as a serious adverse event requiring immediate notification to the sponsor or its designated representative. For all adverse events, sufficient information should be obtained by the investigator to determine the causality of the adverse event. The investigator is required to assess causality. For adverse events with a causal relationship to the investigational product, follow-up by the investigator is required until the event resolves or stabilizes at a level acceptable to the investigator and the sponsor clinical monitor or his/her designated representative.

Serious Adverse Events

A serious adverse event (SAE) is defined as an adverse event that:
Results in death;
Is life threatening (places the subject at immediate risk of death);
Requires in-patient hospitalization or prolongation of existing hospitalization;
Results in persistent or significant disability/incapacity; or
Is a congenital anomaly/birth defect

Important medical events that may not result in death, be life threatening, or require hospitalization may be considered an SAE when, based upon appropriate medical judgment, they may jeopardize the patient and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization. A hospitalization meeting the definition for “serious” is any inpatient hospital admission that includes a minimum of an overnight stay in a health care facility.

Inpatient admission does not include: rehabilitation facilities, hospice facilities, skilled nursing facilities, nursing homes, routine emergency room admissions, same day surgeries (as outpatient/same day/ambulatory procedures), or social admission (e.g., subject has no place to sleep).

Safety will be assessed throughout the study by a qualified physician, physician assistant, or nursing staff. Measurements used to evaluate safety will include history, physical examination, vital signs, clinical laboratory tests, urinalysis, 12-lead ECG, and monitoring for AEs.

Laboratory measurements that deviate clinically significantly from previous measurements (as determined by the investigator) may be repeated. If warranted, additional or more frequent testing than is specified in the protocol should be done to provide adequate documentation of AEs and the resolution of AEs.

For all adverse events, enough information should be obtained by the investigator to determine the causality of the adverse event (e.g., study drug or other illness). The relationship of the adverse event to the study treatment is assessed following the definitions below:

Unrelated: any event that does not follow a reasonable temporal sequence from administration of study drug AND that is likely to have been produced by the patient's clinical state or other modes of therapy administered to the patient.

Unlikely: any event that does not follow a reasonable temporal sequence from administration of study drug OR that is likely to have been produced by the patient's clinical state or other modes of therapy administered to the patient.

Possibly: any reaction that follows a reasonable temporal sequence from administration of study drug OR that follows a known response pattern to the suspected drug AND that could not be reasonably explained by the known characteristics of the patient's clinical state or other modes of therapy administered to the patient.

Related: any reaction that follows a reasonable temporal sequence from administration of study drug AND that follows a known response pattern to the suspected drug AND that recurs with re-challenge, AND OR is improved by stopping the drug or reducing the dose.

(a) Dose-Reduction Procedure for Adverse Event Management

In the event where dose-reduction is used for AE management, two dose reductions are allowed. By 30% of the baseline dose at each dose reduction. Dose reductions are pursued when clinical benefit is expected and may continue to be derived.

(b) Criteria for Discontinuation of Study Treatment

Patients should ordinarily be maintained on study treatment until confirmed radiographic progression. If the patient has radiographic progression but no unequivocal clinical progression and alternate treatment is not initiated, the patient may continue on study treatment, at the investigator's discretion. However, if patients have unequivocal clinical progression without radiographic progression, study treatment should be stopped and patients advised regarding available treatment options.

(c) Ongoing Safety Review

G9.2-17 should be withheld in the event of a serious or life-threatening immune related adverse reaction (IMAR) or one that prompts initiation of systemic steroids, although specific exceptions (e.g., for certain endocrinopathies in clinically stable patients) may be allowed.

Provide a detailed monitoring plan intended to limit the severity and duration of IMARs that occur during combination drug development.

Abraxane is given at 125 mg/m² intravenously over 30-40 minutes on Days 1, 8, and 15 of each 28-day cycle. Gemcitabine is administered on Days 1, 8 and 15 of each 28-day cycle immediately after Abraxane. One or more of the following may be performed based on development of potential adverse event in a patient:
No adjustment is necessary for patients with mild hepatic impairment.
Withhold Abraxane if AST>10×ULN or bilirubin>5×ULN.
Reduce starting dose in patients with moderate to severe hepatic impairment.
Dose reductions or discontinuation may be needed based on severe hematologic, neurologic, cutaneous, or gastrointestinal toxicities.

### Contraindications:

Neutrophil counts of <1,500 cells/mm³
Severe hypersensitivity reaction to Abraxane. Patients with a known hypersensitivity to gemcitabine.

Tables 9-12 below provide exemplary guidance with respect to recommended doses and reduced doses of Abraxane and gemcitabine. See also Abraxane monograph: Abraxis BioScience, LLC. Highlights of Prescribing Information [Internet]. Summit (NJ): Celgene Corporation; 2019 December [cited 2020 May 7].

TABLE 9
Recommendations for Abraxane Starting Dose in Patients with Hepatic
Impairment

| Degree of | SGOT | | | Abraxane Dose in |
|---|---|---|---|---|
| Hepatic | (AST) | | | Pancreatic |
| Impairment | Levels | | Bilirubin Levels | Adenocarcinomaª |
| Mild | <10 × ULN | AND | >ULN to | 125 mg/m² |
| | | | # 1.25 × ULN | |
| Moderate | <10 × ULN | AND | 1.26 to 2 × ULN | not |
| | | | | recommended |
| Severe | <10 × ULN | AND | 2.01 to 5 × ULN | not |
| | | | | recommended |
| | >10 × ULN | OR | >5 ×ULN | not |
| | | | | recommended |

ªPatients with bilirubin levels above the upper limit of normal were excluded from clinical trials for pancreatic or lung cancer.

TABLE 10
Abraxane and Gemcitabine Dose Level Reductions for Patients with
Adenocarcinoma of the Pancreasⁱ

| | Abraxane Dose | Gemcitabine |
|---|---|---|
| Dose Level | (mg/m²) | (mg/m²) |
| Full dose | 125 | 1000 |
| 1^{st} dose reduction | 100 | 800 |
| 2^{nd} dose reduction | 75 | 600 |
| If additional dose | Discontinue | Discontinue |
| reduction required | | |

TABLE 11
Abraxane and Gemcitabine Dose Recommendation and Modifications for
Neutropenia and/or Thrombocytopenia at the Start of a Cycle or within a
Cycle for Patients with Adenocarcinoma of the Pancreasi

| Cycle | ANC* | | Platelet Count | |
|---|---|---|---|---|
| Day | (cells/mm²) | | (cells/mm²) | Abraxane/Gemcitabine |
| Day 1 | <1500 | OR | <100,000 | Delay doses until |
| | | | | recovery |
| Day 8 | 500 to <1000 | OR | 50,000 to <75,000 | Reduce 1 dose level |
| | <500 | OR | <50,000 | Withhold doses |

Day 15: IF Day 8 doses were reduced or given without modification:

| | | | |
|---|---|---|---|
| 500 to <1000 | OR | 50,000 to <75,000 | Reduce 1 dose level |
| | | | from Day 8 |
| <500 | OR | <50,000 | Withhold doses |

Day 15: IF Day 8 doses were withheld:

| | | | |
|---|---|---|---|
| ≥1,000 | OR | ≥75,000 | Reduce 1 dose level |
| | | | from Day 1 |
| 500 to <1000 | OR | 50,000 to <75,000 | Reduce 2 dose level |
| | | | from Day 1 |
| <500 | OR | <50,000 | Withhold doses |

ANC = Absolute Neutrophil Count

TABLE 12
Abraxane and Gemcitabine Dose Modifications for Other Adverse Drug
Reactions in Patients with Adenocarcinoma of the Pancreas

| Adverse Drug | | |
|---|---|---|
| Reaction | Abraxane | Gemcitabine |
| Febrile Neutropenia: | Withhold until fever resolves and | |
| Grade 3 or 4 | ANC ≥500; resume at next | |
| | lower dose level | |
| Peripheral Neuropathy: | Withhold until improves to | No dose |
| Grade 3 or 4 | #Grade 1; resume at next | reduction |
| | lower dose level | |

| | |
|---|---|
| Cutaneous Toxicity: | Reduce to next lower dose level; |
| Grade 2 or 3 | discontinue treatment if toxicity persists |
| Gastrointestinal Toxicity: | Withhold until improves to #Grade 1; |
| Grade 3 mucositis or diarrhea | resume at next lower dose level |

Given the possibility of extravasation, it is advisable to closely monitor the infusion site for possible infiltration during drug administration. Limiting the infusion of Abraxane to 30 minutes, as directed, reduces the likelihood of infusion-related reactions

(d) Identification of Potential Safety Issues

Dose Limiting Toxicity (DLT) period: One (1) cycle.

One cycle encompasses C1D1 (cycle one day one) and C1D15 (cycle one day fifteen).

Monitoring Plans

In Part 1, the dose-escalation phase, dose escalation to the next cohort will proceed following review of Cycle 1 of each cohort. Safety and available PK data will be used to assess for DLTs in all patients of each cohort by the SMC. As a safety precaution, during dose escalation, new patients will be entered and treated only after the first patient of each cohort has been treated with G9.2-17 and at a minimum 7-14 days post-treatment has elapsed. Select DLT safety analysis for each patient will be performed following completion of Cycle 1. During the expansion phase, toxicities will be monitored by the SMC, which will convene to review aggregate toxicity rate prior to each dose escalation. The frequency of SMC meetings will increase as warranted by an increased toxicity rate. The SMC has the right to recommend to terminate or alter the study design of this clinical study at any time, including but not limited to testing of intermediate dose levels or initiation of the intermittent dose schedule.

(e) Dose Limiting Toxicity Criteria

Dose-limiting toxicity (DLT) is defined as a clinically significant non-hematologic adverse event or abnormal laboratory value assessed as unrelated to metastatic tumor disease progression, intercurrent illness, or concomitant medications and is related to the study drug and occurring during the first cycle on study that meets any of the following criteria:
All Grade 4 non-hematologic toxicities of any duration
All Grade 3 non-hematologic toxicities. Exceptions are:
   Grade 3 nausea, vomiting and diarrhea that does not require hospitalization or TPN support and can be managed with supportive care to ≤Grade 2 within 48 hours.
   Grade 3 electrolyte abnormalities that are corrected to ≤Grade 2 within 24 hours.
   Other grade 3 asymptomatic laboratory abnormalities

DLT period includes one (1) cycle, i.e., four (4) weeks. One cycle encompasses the administration of G9.2-17 on days 1 and 15 (C1D1 and C1D15; Cycle 1 Day 1 and Cycle 1 Day 15, respectively).

(f) Dose Delays and Reductions

Any AE≥Grade 3 possible, probably, or definitely related to one or more study drugs will be discussed with the Medical Monitor before continuing with dosing, with the following exceptions, for which no discussion with the Medical Monitor will be required:
Local injection site reactions lasting <72 hours including pain, redness, swelling, induration, or pruritus
Systemic injection reactions lasting <72 hours of fever, myalgia, headache, or fatigue

Where judged appropriate by the Investigator (after discussion with the Medical Monitor) a dose delay may be necessary for ≥Grade 3 adverse events until resolution of the toxicity (to Grade 1 or less).

In Part 2 of the protocol, if 3 more than 3 patients develop a DLT, the dose of G9.2-17 will be reduced to 1 dose below the recommended Phase 2 dose (RP2D)

### (F) RECIST Criteria for Tumor Assessment

At the baseline tumor assessment, tumor lesions/lymph nodes will be categorized as measurable or non-measurable with measurable tumor lesions recorded according to the longest diameter in the plane of measurement (except for pathological lymph nodes, which are measured in the shortest axis). When more than one measurable lesion is present at baseline all lesions up to a maximum of five lesions total (and a maximum of two lesions per organ) representative of all involved organs should be identified as target lesions. Target lesions should be selected on the basis of their size (lesions with the longest diameter). A sum of the diameters for all target lesions will be calculated and reported as the baseline sum diameters.

All other lesions (or sites of disease) including pathological lymph nodes should be identified as non-target lesions and should also be recorded at baseline. Measurements are not required and these lesions should be followed as ‘present’, ‘absent’, or ‘unequivocal progression’.

Disease response (complete response (CR), partial response (PR), stable disease (SD), and progressive disease (PD)) will be assessed as outlined in Appendix 4.

The disease response measures will allow for the calculation of the overall disease control rate (DCR), which includes CR, PR, and SD, the objective response rate (ORR), which includes CR and PR, progression-free survival (PFS), and time to progression (TTP).

### (G) Patient Completion or Withdrawal

Patient Completion

Part 1—Dose Finding:
Patients will receive study drug at one of 5 dose levels every 2 weeks until progression of disease, unacceptable toxicity, or withdrawal from the study development of dose-limiting toxicity (DLT).
Two patients will be dosed, with a maximum available sample size of 24. Dose escalations will only be initiated when approval from the SMC has been received. At each dose escalation, new patients will only be entered and treated after the two patients in the previous cohort has been treated with G9.2-17 and at a minimum 7 days post-treatment has elapsed.
Part 1 will be completed when six consecutive patients have received the same dose and that dose will be identified as the OBD.

Part 2—Tumor-Type Specific Treatment:

An expansion cohort for patients with metastatic PDAC will entail combination treatment of G9.2-17 and gemcitabine/Abraxane. Completion of study will be dependent upon patient response at 3 months and responding patient survival at 12 months.

Part 2—Expansion:

If a promising efficacy signal is identified within one of the five trial arms attributable to the tumor type, an expansion cohort will be launched to confirm the finding. Completion will be as described for Part 2.

Discontinuation from Study Treatment

A patient may be discontinued prior to completion of the study treatment for any of the following reasons:
Dose-limiting toxicity—defined as a clinically significant non-hematologic adverse event or abnormal laboratory value assessed as unrelated to metastatic tumor disease progression, intercurrent illness, or concomitant medications and is related to the study drug and occurring during the first cycle on study that meets any of the following criteria:
   All Grade 4 non-hematologic toxicities of any duration
   All Grade 3 non-hematologic toxicities. Exceptions are as follow:
      Grade 3 nausea, vomiting and diarrhea that does not require hospitalization or TPN support and can be managed with supportive care to ≤grade 2 within 48 hours.
      Grade 3 electrolyte abnormalities that are corrected to ≤grade 2 within 24 hours.
Progressive disease according to RESIST criteria or significant clinical progression at an earlier time point, if judged by the Investigator to be in the patient's best interests
Intercurrent illness that prevents further administration of treatment

### Example 3. A Non-GLP Single-Dose, Range-Finding Intravenous Toxicity Study in Male Sprague Dawley Rats with 1- and 3-Week Postdose Observation Periods

This study evaluated the anatomical endpoints of G9.2-17 IgG4 following a single intravenous bolus administration to Sprague Dawley rats followed by 1-week (terminal) and 3-week (recovery) necropsies on Days 8 and 22. All animals survived to the scheduled necropsies. There were no test article-related macroscopic findings, organ weight changes, or microscopic findings in either the terminal or recovery necropsy animals on this study.

The objective of this non-GLP exploratory, single-dose, range finding, intravenous toxicity study was to identify and characterize the acute toxicities of G9.2-17 IgG4 following intravenous bolus administration over 2 minutes to Sprague Dawley rats followed by 1-week (terminal) and 3-week (recovery) postdose observation periods.

This non-GLP single dose toxicity study was conducted in 24 Sprague Dawley male rats to determine the toxicokinetics and potential toxicity of G9.2-17 IgG4 at different doses in a single administration. Animals were administered either vehicle or 10 mg/kg, 30 mg/kg or 70 mg/kg G9.2-17 IgG4 by slow bolus intravenous injection for at least 2 minutes on Day 1 followed by either a 1-week (terminal, Day 8) or 3-week (recovery, Day 22) period after the dose. Study endpoints included mortality, clinical observations, body weights, and food consumption, clinical pathology (hematology, coagulation, clinical chemistry and urinalysis), toxicokinetic parameters, ADA evaluation and anatomic pathology (gross necropsy, organ weights, and histopathology). Summaries of the experimental design is provided in Table 13 below.

TABLE 13
Experimental Design

| Group | | Dosage Level | |
|---|---|---|---|
| Number | Treatment | (mg/kg) | Number of Male ^{a} |
| 1 | Vehicle ^{b} | 0 | 6 |
| 2 | G9.2-17 IgG4 | 10 | 6 |
| 3 | G9.2-17 IgG4 | 30 | 6 |
| 4 | G9.2-17 IgG4 | 70 | 6 |

^{a} 3 animals/sex/group were euthanized at the Day 8 terminal necropsy; the remaining 3 animals/sex/group were euthanized at the Day 22 recovery necropsy.
^{b} The vehicle was Formulation Buffer (20 mM Tris, 150 mM NaCl, pH 8.0 ± 0.05).

All surviving animals were submitted for necropsy on Day 8 or Day 22. Complete postmortem examinations were performed and organ weights were collected. The organs were weighed from all animals at the terminal and recovery. Tissues required for microscopic evaluation were trimmed, processed routinely, embedded in paraffin, and stained with hematoxylin and eosin.

There were no unscheduled deaths during the course of this study. All animals survived to the terminal or recovery necropsies. Histological changes noted were considered to be incidental findings or related to some aspect of experimental manipulation other than administration of the test article. There was no test article related alteration in the prevalence, severity, or histologic character of those incidental tissue alterations. No G9.2-17 IgG4-related findings were noted in clinical observations, body weights, food consumption, clinical pathology or anatomic pathology. In conclusion, the single intravenous administration of 10, 30, and 70 mg/kg G9.2-17 IgG4 to Sprague Dawley rats was tolerated with no adverse findings. Therefore, under the conditions of this study the NOEL was 70 mg/kg.

### Example 4. A Non-GLP Single-Dose, Range-Finding Intravenous Infusion Toxicity Study of G9.2-17 IgG4 in Cynomolgus Monkeys with a 3-Week Post-Dose Observation Period

This non-GLP single-dose toxicity study was conducted in 8 cynomolgus monkeys to identify and characterize the acute toxicities of G9.2-17 IgG4 administered at different doses as a single dose. Animals (1 male [M]/1 female [F]/group) were administered either vehicle or 30 mg/kg, 100 mg/kg, or 200 mg/kg G9.2-17 IgG4 by 30-minute intravenous (IV) infusion followed by a 3 week post-dose observation period. Study endpoints included: mortality, clinical observations, body weights, and qualitative food consumption; clinical pathology (hematology, coagulation, clinical chemistry, immunophenotyping and galectin 9 expression on leukocyte subsets, and cytokine analysis); toxicokinetic parameters; serum collection for possible anti-drug antibody evaluation (ADA); and soluble galectin-9 analyses; and anatomic pathology (gross necropsy, organ weights, and histopathology).

No G9.2-17 IgG4-related findings were noted in clinical observations, body weights, food consumption, clinical pathology (hematology, clinical chemistry, coagulation, or cytokine analysis), immunophenotyping, galectin-9 expression on leukocyte subsets, soluble galectin-9 or anatomic pathology.

In conclusion, the single intravenous infusion administration of 30, 100, and 200 mg/kg G9.2-17 IgG4 to cynomolgus monkeys was tolerated with no adverse findings. Therefore, under the conditions of this study the No-observed-Adverse-Effect-Level (NOAEL) was 200 mg/kg, the highest dose level evaluated. The study design is shown in Table 14.

TABLE 14
Experimental Design

| | | | | Adjusted Dose | | Dose Volume | | |
|---|---|---|---|---|---|---|---|---|
| | | | | Concentration | | (mL/kg) | | Animal No. |
| Group | | Dose Level | | (mg/mL) | | Necropsy | | Necropsy |
| No. | Treatment | (mg/kg) | | | Males | Day | Females | Day |
| 1 | Vehicle | 0 | 0 | 20 | 1001 | 22 | 1501 | 22 |
| 2 | G9.2-17 | 30 | 1.5 | 20 | 2001 | 22 | 2501 | 22 |
| | IgG4 | | | | | | | |
| 3 | G9.2-17 | 100 | 5 | 20 | 3001 | 22 | 3501 | 22 |
| | IgG4 | | | | | | | |
| 4ª | G9.2-17 | 200 | 10 | 20 | 4001 | 22 | 4501 | 22 |
| | IgG4 | | | | | | | |
| | | Dose | Adjusted Dose | Dose | Animal No. | | | |
| Group | | Level | Concentration | Volume | | Necropsy | | Necropsy |
| No. | Treatment | (mg/kg) | (mg/mL) | (mL/kg) | Males | Day | Females | Day |
| 1 | Vehicle | 0 | 0 | 20 | 1001 | 22 | 1501 | 22 |
| 2 | G9.2-17 | 30 | 1.5 | 20 | 2001 | 22 | 2501 | 22 |
| | IgG4 | | | | | | | |
| 3 | G9.2-17 | 100 | 5 | 20 | 3001 | 22 | 3501 | 22 |
| | IgG4 | | | | | | | |
| 4ª | G9.2-17 | 200 | 10 | 20 | 4001 | 22 | 4501 | 22 |
| | IgG4 | | | | | | | |

^{a}Group 4 was administered 1 week after administration of Groups 1 through 3.

The vehicle and test article were administered once via IV infusion for 30 minutes during the study via a catheter percutaneously placed in the saphenous vein. The dose levels were 30, 100, and 200 mg/kg and administered at a dose volume of 20 mL/kg. The control group received the vehicle in the same manner as the treated groups.

The animals were placed in sling restraints during dosing. The vehicle or test article were based on the most recent body weights and administered using an infusion pump and sterile disposable syringes. The dosing syringes were filled with the appropriate volume of vehicle or test article (20 mL/kg with 2 mL extra). At the completion of dosing, the animals were removed from the infusion system. The weight of each dosing syringe was recorded prior to the start and end of each infusion to determine dose accountability.

Detailed Clinical Observations

The animals were removed from the cage, and a detailed clinical examination of each animal was performed at 1 and 4.5 hours post-start of infusion (SOI) on Day 1 and once daily thereafter during the study. The animals were removed from the cage, and a detailed clinical examination of each animal was performed at 1 and 4.5 hours post-start of infusion (SOI) on Day 1 and once daily thereafter during the study. Body weights for all animals were measured and recorded at transfer, prior to randomization, on Day −1, and weekly during the study.

Clinical pathology evaluations (hematology, coagulation, and clinical chemistry) were conducted on all animals pretest and on Days 1 (prior to dosing), 3, 8, and 21. Additional samples for the determination of hematology parameters and peripheral blood lymphocyte and cytokine analysis samples were collected at 30 minutes (immediately after the end of infusion) and 4.5, 8.5, 24.5, and 72.5 hours post-SOI (relative to Day 1). Bone marrow smears were collected and preserved.

Blood samples (approximately 0.5 mL) were collected from all animals via the femoral vein for determination of the serum concentrations of the test article (see Table 15) (for a deviation, see Appendix 1). The animals were not fasted prior to blood collection, with the exception of the intervals that coincided with fasting for clinical pathology collections.

TABLE 15
Bioanalysis Sample Collection Schedule

| | Sample Collection Time Points | | | | | | | | | | | | |
|---|---|---|---|---|---|---|---|---|---|---|---|---|---|
| | (Time Post-SOI) relative to Day 1 | | | | | | | | | | | | |
| | | | | | | | 24.5 | 48.5 | 72.5 | 120.5 | 168.5 | 360.5 | 504.5 |
| Group | | 0.583 | 1 | 2.5 | 4.5 | 8.5 | hr | hr | hr | hr | hr | hr | hr |
| No. | Predose | hr^{a} | hr | hr | hr | hr | (Day 2) | (Day 3) | (Day 4) | (Day 6) | (Day 8) | (Day 16) | (Day 22) |
| 1-4 | X | X | X | X | X | X | X | X | X | X | X | X | X |

X = Sample was collected.
^{a}Only the 0.583 hr post-SOI timepoint from Group 1 animals was analyzed for test article content. Additional timepoints may be analyzed at the discretion of the Study Director.

For processing, blood samples were collected in non-additive barrier free microtubes and centrifuged at controlled room temperature within 1 hour of collection. The resulting serum was divided into 2 approximately equal aliquots in pre labeled cryovials. All aliquots were stored frozen at −60° C. to −90° C. within 2 hours of collection.

Postmortem study evaluations were performed on all animals euthanized at the scheduled necropsy.

Necropsy examinations were performed under procedures approved by a veterinary pathologist. The animals were examined carefully for external abnormalities including palpable masses. The skin was reflected from a ventral midline incision and any subcutaneous masses were identified and correlated with antemortem findings. The abdominal, thoracic, and cranial cavities were examined for abnormalities. The organs were removed, examined, and, where required, placed in fixative. All designated tissues were fixed in neutral buffered formalin (NBF), except for the eyes (including the optic nerve) and testes. The eyes (including the optic nerve) and testes were placed in a modified Davidson's fixative, and then transferred to 70% ethanol for up to three days prior to final placement in NBF. Formalin was infused into the lung via the trachea. A full complement of tissues and organs was collected from all animals.

Body weights and protocol-designated organ weights were recorded for all animals at the scheduled necropsy and appropriate organ weight ratios were calculated (relative to body and brain weights). Paired organs were weighed together. A combined weight for the thyroid and parathyroid glands was collected.

Results

All animals survived to the scheduled necropsy on Day 22. No test article-related clinical or veterinary observations were noted in treated animals. No test article-related effects on body weight were observed in treated animals during the treatment or recovery period. There were no G9.2-17 IgG4-related effects on hematology endpoints in either sex at any dose level at any interval.

There were no G9.2-17 IgG4-related effects on coagulation times (i.e., activated partial thromboplastin times [APTT] and prothrombin times) or fibrinogen concentrations in either sex at any dose level at any interval. All fluctuations among individual coagulation values were considered sporadic, consistent with biologic and procedure-related variation, and/or negligible in magnitude, and not related to G9.2-17 IgG4 administration.

There were no G9.2-17 IgG4-related effects on clinical chemistry endpoints in either sex at any dose level at any interval. All fluctuations among individual clinical chemistry values were considered sporadic, consistent with biologic and procedure-related variation, and/or negligible in magnitude, and not related to G9.2-17 IgG4 administration.

There were no G9.2-17 IgG4-related effects on cytokine endpoints in either sex at any dose level at any interval. All fluctuations among individual cytokine values were considered sporadic, consistent with biologic and procedure-related variation, and/or negligible in magnitude, and not related to G9.2-17 IgG4 administration.

Review of the gross necropsy observations revealed no findings that were considered to be test article related. There were no organ weight alterations that were considered to be test article-related. There were no test article-related changes.

In conclusion, the single intravenous infusion administration of 30, 100, and 200 mg/kg G9.2-17 IgG4 to cynomolgus monkeys was tolerated with no adverse findings. Therefore, under the conditions of this study the No-observed-Adverse-Effect-Level (NOAEL) was 200 mg/kg, the highest dose level evaluated.

The animals were removed from the cage, and a detailed clinical examination of each animal was performed at 1 and 4.5 hours post-start of infusion (SOI) on Day 1 and once daily thereafter during the study.

### Example 5. Intravenous Infusion Study of G9.2-17 in Cynomolgus Monkeys

The objective of this study was to further characterize the toxicity and toxicokinetics of the test article, G9.2-17 (a hIgG4 Monoclonal Antibody which binds to Galectin-9) at different doses, following once weekly 30-minute intravenous (IV) infusion for 5 weeks in cynomolgus monkeys, and to evaluate the reversibility, progression, or delayed appearance of any observed changes following a 3-week recovery period.

### Experimental Design

Table 16 summarizes the study design.

TABLE 16
Experimental Design

| | | | Dose | Dose | Main Study | | Recovery Study | |
|---|---|---|---|---|---|---|---|---|
| Group | Test | Dose Level | Volume^{a} | Concentration | No. of | No. of | No. of | No. of |
| No. | Material | (mg/kg/dose) | (mL/kg) | (mg/mL) | Males | Females | Males | Females |
| 1 | Vehicle | 0 | 10 | 0 | 3 | 3 | 2 | 2 |
| 2 | G9.2-17 | 100 | 10 | 10 | 3 | 3 | 2 | 2 |
| 3 | G9.2-17 | 300 | 10 | 30 | 3 | 3 | 2 | 2 |

^{a}Based on the most recent practical body weight measurement.

Animals (cynomolgus monkeys) used in the study were assigned to study groups by a standard, by weight, randomization procedure designed to achieve similar group mean body weights. Males and females were randomized separately. Animals assigned to study had body weights within ±20% of the mean body weight for each sex.

The formulations lacking G9.2-17 (“vehicle”) or encompassing G9.2-17 (“test article”) were administered to the animals once weekly for 5 weeks (Days 1, 8, 15, 22, and 29) during the study via 30-minute IV infusion. The dose levels were 0, 100 and 300 mg/kg/dose and administered at a dose volume of 10 mL/kg. The control animals group received the vehicle in the same manner as the treated groups. Doses were administered via the saphenous vein via a percutaneously placed catheter and a new sterile disposable syringe was used for each dose. Dose accountability was measured and recorded prior to dosing and at the end of dosing on toxicokinetic sample collection days (Days 1, 15, and 29) to ensure a ±10% target dose was administered. Individual doses were based on the most recent body weights. The last dose site was marked for collection at the terminal and recovery necropsies. All doses were administered within 8 hours of test article preparation.

In-life procedures, observations, and measurements were performed on the animals as exemplified below.

Electrocardiographic examinations were performed on all animals. Insofar as possible, care was taken to avoid causing undue excitement of the animals before the recording of electrocardiograms (ECGs) in order to minimize extreme fluctuations or artifacts in these measurements. Standard ECGs (10 Lead) were recorded at 50 mm/sec. Using an appropriate lead, the RR, PR, and QT intervals, and QRS duration were measured and heart rate was determined. Corrected QT (QTc) interval was calculated using a procedure based on the method described by Bazett (1920). All tracings were evaluated and reported by a consulting veterinary cardiologist.

To aid in continuity and reliability, functional observational battery (FOB) evaluations were conducted by two independent raters for all occasions and consisted of a detailed home cage and open area neurobehavioral evaluation (Gauvin and Baird, 2008). Each technician scored the monkey independently (without sharing the results with each other) for each home cage and out of cage observational score, and then the individual scores were assessed for agreement with their partner's score after the completion of the testing. FOB evaluations were conducted on each animal predose (on Day −9 or Day 8) to establish baseline differences and at 2 to 4 hours from the start of infusion on Days 1 and 15, and prior to the terminal and recovery necropsies. The observations included, but were not limited to, evaluation of activity level, posture, lacrimation, salivation, tremors, convulsions, fasciculations, stereotypic behavior, facial muscle movement, palpebral closure, pupil response, response to stimuli (visual, auditory, and food), body temperature, Chaddock and Babinski reflexes, proprioception, paresis, ataxia, dysmetria, and slope assessment, movement, and gait.

Blood pressure of each animal was measured and recorded and consisted of systolic, diastolic, and mean arterial pressure. Blood pressure measurements are reported using three readings that have the Mean Arterial Pressure (MAP) within 20 mmHg.

Respiratory rates of each animal were measured and recorded 3 times per animal/collection interval by visual assessment per Testing Facility SOP. The average of the 3 collections is the reported value.

Clinical pathology evaluations (e.g., immunophenotyping and cytokine evaluations) were conducted on all animals at predetermined intervals. Bone marrow smears were collected and preserved. Blood samples (approximately 0.5 mL) were collected from all animals via the femoral vein for determination of the serum concentrations of the test article. The animals were not fasted prior to blood collection, with the exception of the intervals that coincided with fasting for clinical pathology collections. At the conclusion of the study (day 36 or day 50), animals were euthanatized and tissues for histology processing and microscopic evaluation were collected.

Soluble galectin-9 was evaluated as follows. Blood samples (approximately 1 mL) were collected from all animals via the femoral vein for determination of the serum for soluble galectin 9 predose and 24 hours from the start of infusion on Days 1, 8, 15, and 29, and prior to the terminal and/or recovery necropsies. The animals were not fasted prior to blood collection, with the exception of the intervals that coincided with fasting for clinical pathology collections.

Soluble galectin-9 samples were processed as follows. Blood samples were collected in non-additive, barrier free tubes, allowed to clot at ambient temperature, and centrifuged at ambient temperature. The resulting serum was divided into 2 aliquots (100 μL in Aliquot 1 and remaining in Aliquot 2) in pre labeled cryovials. All aliquots were flash frozen on dry ice within 2 hours of collection and stored frozen at −60° C. to 90° C.

All results presented in the tables of the report were calculated using non-rounded values as per the raw data rounding procedure and may not be exactly reproduced from the individual data presented.

### Results

Mortality

All animals survived to the scheduled terminal necropsy on Day 36 and recovery necropsy on Day 50.

Detailed Clinical and Veterinary Observations

No test article-related clinical or veterinary observations were noted in treated animals during the treatment or recovery periods.

Functional Observational Battery

No test article-related FOB observations were noted in treated animals during the treatment or recovery periods.

Body Weight and Body Weight Gains

No test article-related effects in body weight and body weight gain were noted in treated animals during the treatment or recovery periods.

Ophthalmology Examinations

No test article-related effects in ophthalmology examinations were noted in treated animals during the treatment or recovery periods.

Blood Pressure Values

No test article-related effects in blood pressure values were noted in treated animals during the treatment or recovery periods.

Respiratory Rate Values

No test article-related effects in respiratory rate values were noted in treated animals during the treatment or recovery periods.

Electrocardiology

No test article-related effects in electrocardiographic evaluations were noted in treated animals during the treatment or recovery periods.

Hematology

There were no G9.2-17-related effects among hematology parameters in either sex at any dose level at any timepoint.

Coagulation

There were no G9.2-17-related effects among coagulation parameters in either sex at any dose level at any timepoint.

Clinical Chemistry

There were no G9.2-17-related effects among clinical chemistry parameters in either sex at any dose level at any timepoint.

Urinalysis

No G9.2-17-related alterations were observed among urinalysis parameters in either sex at any dose level at the 13-week interim.

Cytokine

No definitive G9.2-17-related effects on cytokines were seen at any dose level or timepoint.

Peripheral Blood Leukocyte Analysis (PBLA)

There were no G9.2-17-related effects on PBLA endpoints in either sex at any dose level at any timepoint.

Bioanalysis, Galectin-9, and Toxicokinetic Evaluation

G9.2-17 was quantifiable in all cynomolgus monkey samples from all G9.2-17-dosed animals after dose administration. No measurable amount of G9.2-17 was detected in control cynomolgus monkey samples. Soluble galectin-9 was quantifiable in all cynomolgus monkey samples from all animals. G9.2-17 serum concentrations were below the bioanalytical limit of quantitation (LLOQ<0.04 ug/mL) in all serum samples obtained predose from most G9.2-17 treated animals on Day 1 and from control animals on Days 1 and 29.

Gross Pathology and Organ Weight

There were no definitive test article-related macroscopic observations in main study or recovery animals. There were also no test article-related organ weight changes for main study or recovery animals.

Histopathology

There were no definitive test article-related microscopic observations.

In conclusion, once weekly intravenous infusion administration of 100 and 300 mg/kg of G9.2-17 for 5-weeks to cynomolgus monkeys was tolerated with no adverse findings.

### Example 6. Intravenous Infusion Study of G9.2-17 in Sprague Dawley Rats

The objective of this study was to evaluate potential toxicity of G9.2-17, an IgG4 human monoclonal antibody directed against galectin-9 at different doses, when administered by intravenous injection to Sprague Dawley Rats once weekly for 4 consecutive weeks followed by a 3-week post dose recovery period. In addition, the toxicokinetic characteristics of G9.2-17 were determined.

### Experimental Design

Table 17 summarizes the study design.

TABLE 17
Study Design

| | | Dose | Dose | Dose | | | | | | |
|---|---|---|---|---|---|---|---|---|---|---|
| | Test | Level | Concentration | Volumeª | Terminal | | Recovery | | TK/Gal-9/Cyto | |
| Group | Material | (mg/kg) | (mg/mL) | (mL/kg) | M | F | M | F | M | F |
| 1 | Control | 0 | 0 | 10 | 10 | 10 | 5 | 5 | 12 | 12 |
| 2 | G9.2-17 | 100 | 10 | 10 | 10 | 10 | 5 | 5 | 12 + 6^{b} | 12 + 6^{b} |
| 3 | G9.2-17 | 300 | 30 | 10 | 10 | 10 | 5 | 5 | 12 + 6^{b} | 12 + 6^{b} |

^{a}Individual dose volumes were calculated based on the most recent body weight.
^{b}SSD animals: 3 animals/sex/group for TK collections only following a single dose administration on Day 1.

One hundred eighty-six animals (Sprague Dawley rats) were assigned to treatment groups randomly by body weight. Control Article/Vehicle, Formulation Buffer for Test Article, and test article, G9.2-17, were administered via a single IV injection in a tail vein at dose levels of 0, 100, and 300 mg/kg once on Days 1, 8, 15, 22, and 29. Test article was administered at dose levels of 100 and 300 mg/kg once on Day 1 to animals assigned to the SSD subgroup.

Clinical observations were performed once daily prior to room cleaning in the morning, beginning on the second day of acclimation. A mortality check was conducted twice daily to assess general animal health and wellness. Food consumption was estimated by weighing the supplied and remaining amount of food in containers once weekly. The average gram (g)/animal/day was calculated from the weekly food consumption. Body weights were taken prior to randomization, on Day −1, then once weekly throughout the study, and on the day of each necropsy. Functional Observation Battery (FOB) observations were recorded for SSB animals approximately 24 hours post dose administrations on Days 1, 35 and 49. Urine was collected overnight using metabolic cages. Samples were obtained on Days 36 and 50.

Animals were fasted overnight prior to each series of collections that included specimens for serum chemistry. In these instances, associated clinical pathology evaluations were from fasted animals. Blood was collected from a jugular vein of restrained, conscious animals or from the vena cava of anesthetized animals at termination.

Parameters assessed during the In-life examinations of the study included clinical observations, food consumption, body weights, functional observational battery. Blood samples were collected at selected time points for clinical pathology (hematology, coagulation, and serum chemistry) analyses. Urine samples were collected for urinalysis. Blood samples were also collected at selected time points for toxicokinetic (TK), immunogenicity (e.g., anti-drug antibody or ADA), and cytokine analyses. Animals were necropsied on Days 36 and 50. At each necropsy, gross observations and organ weights were recorded, and tissues were collected for microscopic examination.

### Results

### In-Life Examinations

Mortality: There were no abnormal clinical observations or body weight changes noted for this animal during the study.

Clinical Observations: There were no G9.2-17-related clinical observations noted during the study.

Food Consumption/Body Weights: There were no G9.2-17-related changes in food consumption, body weights or body weight gain noted during the study.

Clinical Pathology: There were no G9.2-17-related changes noted in clinical pathology parameters.

Cytokine Analysis: There were no G9.2-17-related changed in serum concentrations of IL-2, IL-4, IFN-7, IL-5, IL-6, IL-10, and/or TNF-α, MCP-1 and MIP-1b.

Gross Pathology: There were no G9.2-17-related gross observations. Further, were no G9.2-17-related changes in absolute or relative organ weights.

Histopathology: There were no G9.2-17-related histologic findings.

In conclusion, intravenous G9.2-17 administration to Sprague Dawley rats once weekly for a total of 5 doses was generally well tolerated. There were no G9.2-17-related changes in clinical observations, food consumption, body weights, FOB parameters, clinical pathology, cytokine, gross observations, or organ weights.

### Example 7. Inhibition of Polarization and Repolarization of M2 Macrophages

Macrophages play an indispensable role in the immune system with decisive functions in both innate and acquired immunity. M1 macrophages are generally considered potent effector cells which can kill tumor cells, while M2 polarized macrophages express a series of cytokines, chemokines, and proteases to promote angiogenesis, lymphangiogenesis, tumor growth, metastasis, and immunosuppression (Sica et al., 2008; Semin. Cancer Biol. 2008; 18: 349-355). In M2 macrophages, production of anti-inflammatory cytokines, such as TGF-β and IL-10, is enhanced (Martinez et al., Front Biosci. 2008 Jan. 1; 13:453-61., Mantovani et al., Trends Immunol 2002 November; 23(11):549-55.; Zhang et al., J Hematol Oncol 10, 58 (2017)). Given that macrophages comprise a key component of the host immune response, inhibition of polarization or repolarization of M2 macrophages is an important therapeutic consideration in oncological immunotherapy (Poh and Ernst, Front Oncol. 2018 Mar. 12; 8:49).

Whole blood from three healthy human donors was used to isolate CD14+ monocytes. The monocytes were allowed to differentiate to macrophages in X-VIVO-15 media (Lonza) in a 10 cm tissue culture dish for 7 days. The differentiated macrophages were either used directly for assessing inhibition of polarization, or they were cryopreserved and used at a later time for repolarization assays. Prior to use in an assay, the M0 macrophages were phenotyped.

Two different polarization cocktails were used to evaluate macrophage polarization: one with a mixture of IL-4 and IL-13, and a second containing only gal-9. The effect of G9.2-17 on M2 polarization was tested via its direct addition to one of these cocktails, and incubation with macrophages for 48 hours. The effect of G9.2-17 on repolarization of M2 macrophages was tested via addition to the M2-polarized macrophages.

The state of polarization was identified by the measurement of secretion of either IL-10 (repolarization) or TGF-beta1 (inhibition of polarization and repolarization). These factors were quantified in cell culture supernatants using CytoMetric Bead Arrays following the manufacturer's protocol.

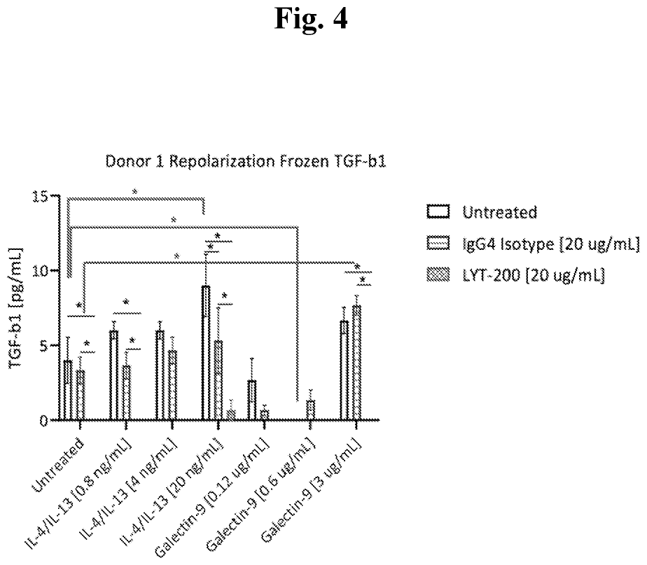

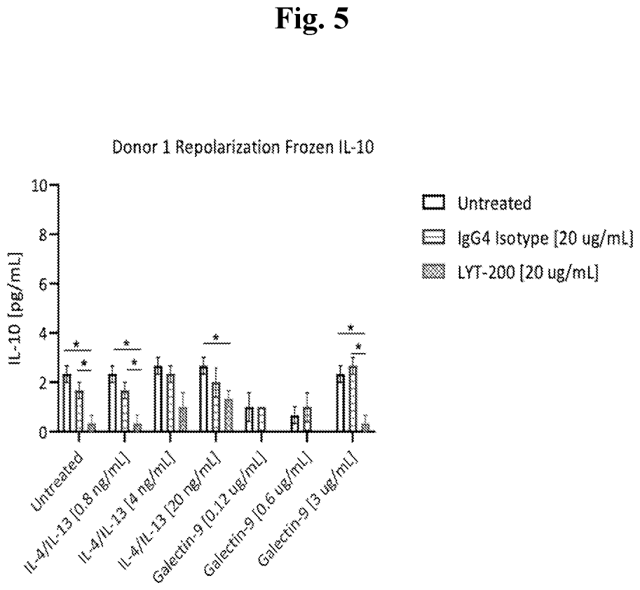

Representative data from one donor showing the effect of G9.2-17 on polarization of fresh monocyte-derived macrophages is in FIG. 4. All donor macrophages showed similar results, with a decrease in TGF-beta1 secretion following incubation with G9.2-17 compared to the isotype matched control or untreated cells. FIG. 4 shows the effect on TGF-beta1 secretion by previously frozen macrophages following incubation with G9.2-17 or an isotype matched control. Treatment with 20 ng/mL of polarization cocktail significantly induced TGF-β1 secretion, while G9.2-17 treatment abolished the IL-4/IL-13-dependent increase of TGF-β1 secretion. FIG. 5 shows the effects on IL-10 secretion on repolarization of cryopreserved macrophages. Treatment with G9.2-17 led to a reduction of secreted IL-10 and TGF-b1 levels in all donors compared to untreated and IgG4 isotype control antibody controls, in the presence of both types of polarization cocktails.

This assay confirms that G9.2-17 can potently inhibit TGF-beta1 and IL-10 at the concentration of 20 μg/ml.

### EQUIVALENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art are readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art are readily appreciate that all parameters, dimensions, materials, and configurations described herein are meant to be exemplary and that the actual parameters, dimensions, materials, and/or configurations are depend upon the specific application or applications for which the inventive teachings is/are used. Those skilled in the art are recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” are refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for treating a solid tumor, comprising administering to a subject in need thereof an effective amount of an antibody that binds human galectin-9 (anti-Gal9 antibody), wherein the anti-Gal9 antibody has the same heavy chain complementarity determining regions (CDRs) and the same light chain CDRs as antibody G9.2-17; wherein the subject is undergoing an anti-cancer therapy comprising one or more chemotherapeutics, and wherein the subject has one or more of the following features:
(i) has no resectable cancer;
(ii) has no infection by SARS-CoV-2; and
(iii) has no active brain or leptomeningeal metastasis.

2. A method for treating a solid tumor, comprising administering to a subject in need thereof an effective amount of an antibody that binds human galectin-9 (anti-Gal9 antibody) and an effective amount of one or more chemotherapeutics; wherein the anti-Gal9 antibody has the same heavy chain complementarity determining regions (CDRs) and the same light chain CDRs as antibody G9.2-17, and wherein the subject has one or more of the following features:
(i) has no resectable cancer;
(ii) has no infection by SARS-CoV-2; and
(iii) has no active brain or leptomeningeal metastasis.

3. A method for treating a solid tumor, comprising administering to a subject in need thereof an effective amount of one or more chemotherapeutics; wherein the subject is undergoing a therapy comprising an antibody that binds human galectin-9 (anti-Gal9 antibody), which has the same heavy chain complementarity determining regions (CDRs) and the same light chain CDRs as antibody G9.2-17, and wherein the subject has one or more of the following features:
(i) has no resectable cancer;
(ii) has no infection by SARS-CoV-2; and
(iii) has no active brain or leptomeningeal metastasis.

4. The method of any one of claims 1-3, wherein the solid tumor is a metastatic solid tumor.

5. The method of any one of claims 1-4, wherein the solid tumor is pancreatic ductal adenocarcinoma (PDAC), and wherein the subject has no locally advanced PDAC without distant organ metastatic deposits.

6. The method of any one of claims 1-5, wherein the one or more chemotherapeutics comprise an antimetabolite, a microtubule inhibitor, or a combination thereof.

7. The method of claim 6, wherein the antimetabolite is gemcitabine and/or the microtubule inhibitor is paclitaxel.

8. The method of any one of claims 1, 2, and 4-7, wherein the anti-Gal9 antibody is administered to the subject at a dose of about 0.5 mg/kg to about 32 mg/kg once every two weeks by intravenous injection.

9. The method of any one of claims 1, 2, and 4-8, wherein the anti-Gal9 antibody is administered to the subject at a dose of about 2 mg/kg to about 16 mg/kg once every two weeks by intravenous injection.

10. The method of claim 9, wherein the anti-Gal9 antibody is administered to the subject at a dose of about 2 mg/kg, about 4 mg/kg, about 8 mg/kg, about 12 mg/kg, or about 16 mg/kg once every two weeks by intravenous injection.

11. The method of any one of claims 7-10, wherein the method comprises a cycle of 28 days, in which the anti-Gal9 antibody is administered to the subject on day 1 and day 15 and the gemcitabine and paclitaxel are administered to the subject on day 1, day 8, and day 15.

12. The method of claim 11, wherein the paclitaxel is a protein-bound paclitaxel, which preferably is a nanoparticle albumin-bound paclitaxel.

13. The method of claim 11 or claim 12, wherein the paclitaxel is administered to the subject at 125 mg/m² intravenously.

14. The method of any one of claims 7-13, wherein the gemcitabine is administered to the subject at 1000 mg/m².

15. The method of any one of claims 7-14, wherein the anti-Galectin-9 antibody comprises a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3 and/or comprises a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6

16. The method of any one of claims 1-15, wherein the anti-Gal9 antibody comprises a heavy chain variable region (V_{H}) that comprises the amino acid sequence of SEQ ID NO: 7; and a light chain variable region (V_{L}) that comprises the amino acid sequence of SEQ ID NO: 8.

17. The method of any one of claims 1-16, wherein the anti-Gal9 antibody is an IgG4 molecule.

18. The method of claim 17, wherein the anti-Gal9 antibody comprises a heavy chain that comprises the amino acid sequence of SEQ ID NO: 19 and a light chain that comprises the amino acid sequence of SEQ ID NO: 15.

19. The method of any one of claims 1-18, wherein the subject is a human patient.

20. The method of any one of claims 1-19, wherein the subject comprises galectin-9 positive cancer cells or immune cells.

21. The method of claim 20, wherein galectin-9 positive cancer cells or immune cells are detected in tumor organoids derived from the subject.

22. The method of any one of claims 1-21, wherein the subject has an elevated level of galectin-9 relative to a control value.

23. The method of claim 22, wherein the subject has an elevated serum or plasma level of galectin-9 relative to the control value.

24. The method of any one of claims 1-23, wherein the subject received at least one line of systemic anti-cancer therapy.

25. The method of any one of claims 1-24, wherein the subject is free of prior therapy involving gemcitabine and/or paclitaxel or had a prior therapy involving gemcitabine and/or paclitaxel at least six months before administration of the anti-Gal9 antibody.

26. The method of any one of claims 1-25, wherein the subject is examined for one or more of the following features before, during, and/or after the treatment:
(a) one or more tumor markers in tumor biopsy samples from the subject, optionally wherein the one or more tumor markers comprise CA15-3, CA-125, CEA, CA19-9, and/or alpha fetoprotein;
(b) cytokine profile; and
(c) galectin 9 levels.

27. The method of any one of claims 1-26, wherein the method further comprises monitoring occurrence of one or more adverse effects in the subject.

28. The method of claim 27, wherein the one or more adverse effects comprise hepatic impairment, hematologic toxicity, neurologic toxicity, cutaneous toxicity, gastrointestinal toxicity, or a combination thereof.

29. The method of claim 27 or claim 28, further comprising reducing the dose of the anti-Gal9 antibody, the dose of the one or more chemotherapeutics, or both, when an adverse effect is observed.

30. The method of claim 29, wherein administration of the paclitaxel is withheld when the subject has a level of aspartate transaminase (AST) greater than 10× upper limit of normal (ULN), a level of bilirubin greater than 5×ULN, or both.

31. The method of claim 30, wherein the method further comprises reducing the dose of the anti-Gal9 antibody, the dose of the gemcitabine, the dose of the paclitaxel, or a combination thereof, when moderate to severe hepatic impairment is observed.

32. The method of claim 31, wherein the method further comprises reducing the dose or terminating administration of the anti-Gal9 antibody, the gemcitabine, the paclitaxel, or a combination thereof, when severe hematologic toxicity, neurologic toxicity, cutaneous toxicity, and/or gastrointestinal toxicity is observed.

33. The method of claim 31 or claim 32, wherein the dose of the paclitaxel is reduced to 100 mg/m²-75 mg/m².

34. The method of any one of claims 31-33, wherein the dose of the gemcitabine is reduced to 800 mg/m²-600 mg/m².


SEQUENCE LISTING (23 sequences)

SEQ ID NO: 1 - protein, 11 aa, Artificial Sequence
  Synthetic
RASQSVSSAVA

SEQ ID NO: 2 - protein, 7 aa, Artificial Sequence
  Synthetic
SASSLYS

SEQ ID NO: 3 - protein, 9 aa, Artificial Sequence
  Synthetic
QQSSTDPIT

SEQ ID NO: 4 - protein, 9 aa, Artificial Sequence
  Synthetic
FTVSSSSIH

SEQ ID NO: 5 - protein, 17 aa, Artificial Sequence
  Synthetic
YISSSSGYTYYADSVKG

SEQ ID NO: 6 - protein, 15 aa, Artificial Sequence
  Synthetic
YWSYPSWWPYRGMDY

SEQ ID NO: 7 - protein, 124 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSS

SEQ ID NO: 8 - protein, 108 aa, Artificial Sequence
  Synthetic
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKR

SEQ ID NO: 9 - protein, 20 aa, Artificial Sequence
  Synthetic
MYRMQLLSCIALSLALVTNS

SEQ ID NO: 10 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 106 aa, Artificial Sequence
  Synthetic
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, Artificial Sequence
  Synthetic
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 454 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK